(19) United States
(12) Patent Application Publication
(10) Pub. No.: US 2016/0183973 A1
(43) Pub. Date: Jun. 30, 2016

(54) MEDICAL TOOL AND PUNCTURE METHOD

(71) Applicant: TERUMO KABUSHIKI KAISHA, Tokyo (JP)

(72) Inventors: Masakatsu KAWAURA, Sunnyvale, CA (US); Nao MANDAI, Mountain View, CA (US)

(73) Assignee: TERUMO KABUSHIKI KAISHA, Tokyo (JP)

(21) Appl. No.: 15/059,479

(22) Filed: Mar. 3, 2016

Related U.S. Application Data
(63) Continuation of application No. PCT/JP2014/074102, filed on Sep. 11, 2014.

(30) Foreign Application Priority Data
Sep. 20, 2013 (JP) 2013-196171

(51) Int. Cl. A61B 17/34; A61F 2/00

(52) U.S. Cl. CPC A61B 17/3468; A61F 2/0045; A61F 2002/0072; A61B 2217/005

(57) ABSTRACT

A medical tool is disclosed, which includes a tensioning portion configured to apply, before a puncture needle puncturing a living body tissue forms, in the living body tissue, an insertion hole into which an implant can be inserted, force to a portion through which the puncture needle is to pass in a direction different from a direction in which the puncture needle punctures the living body tissue to establish a tensioned state.

### CROSS-REFERENCES TO RELATED APPLICATIONS

This application is a continuation of International Application No. PCT/JP2014/074102 filed on Sep. 11, 2014, and claims priority to Japanese Application No. 2013-196171 filed on Sep. 20, 2013, the entire content of both of which is incorporated herein by reference.

### TECHNICAL FIELD

The present disclosure generally relates to a medical tool and a puncture method.

### BACKGROUND DISCUSSION

If a person suffers from a urinary incontinence, specifically, for example, if a person suffers from a stress urinary incontinence, then urine leakage can be caused by application of abdominal pressure during normal exercise or by laughing, coughing, sneezing or the like. The cause of this may be, for example, that the pelvic floor muscle which is a muscle for supporting the urethra is loosened by birth.

For the treatment of urinary incontinence, a surgical treatment can be effective, in which there is used, for example, a belt-shaped living body tissue supporting indwelling article called “sling.” The sling is indwelled inside the body and the urethra is supported by the sling (for example, refer to Japanese Patent Laid-Open No. 2010-99499). In order to indwell the sling inside the body, an operator would incise the vagina with a surgical knife, dissect the part between the urethra and vagina, and make the dissected region and the outside communicate with each other through obturator foramens by use of a puncture needle or the like. Then, in this state, the sling is indwelled into the body.

If the vagina is incised once, however, a situation may occur that the sling is exposed to the inside of the vagina from a wound caused by the incision, and complications may be caused by an infection from the wound or the like. Further, since the vagina is incised, there is such a defect that the invasion is relatively great and the burden on the patient is relatively heavy. Further, the urethra may be damaged in the course of the procedure by the operator. In addition, the fingertip of the operator himself/herself may be damaged.

### SUMMARY

A medical tool and a puncture method are disclosed by which an implant can be embedded relatively easily into a living body while the burden on a patient can be reduced and the safety of a patient can be improved.

In order to attain the object described above, according to an aspect of the present disclosure, a medical tool is disclosed, which includes a tensioning portion configured to apply, before a puncture needle puncturing a living body tissue forms, in the living body tissue, an insertion hole into which an implant can be inserted, force to a portion through which the puncture needle is to pass in a direction different from a direction in which the puncture needle punctures the living body tissue to establish a tensioned state.

In accordance with an exemplary embodiment, preferably, the medical tool is configured such that the tensioning portion includes an attachment portion configured to be attached to a peripheral portion of a portion at which puncture by the puncture needle is to be started to press the peripheral portion to tension the portion at which puncture by the puncture needle is to be started.

In accordance with an exemplary embodiment, preferably, the medical tool is configured such that a plurality of attachment portions press the peripheral portion individually in directions different from each other.

Alternatively, the medical tool may be configured such that the attachment portion is configured from a tongue having elasticity.

In accordance with an exemplary embodiment, preferably, the tensioning portion may have a suction portion configured to suck a portion through which the puncture needle is to pass.

In accordance with an exemplary embodiment, preferably, the medical tool may further include a deformation suppressing portion configured to be attached to a living body surface to prevent and suppress deformation of the living body tissue at a peripheral portion of a portion at which the puncture needle is to pass the living body.

In accordance with an exemplary embodiment, preferably, the medical tool is configured such that the deformation suppressing portion is configured from an elastic body configured to be attached, after the puncture needle punctures the living body tissue, again to the living body tissue at a peripheral portion of a portion at which the puncture needle is exposed from the living body surface.

According to another aspect of the present disclosure, a puncture method is disclosed for puncturing a living body tissue by a puncture needle, which is capable of puncturing the living body tissue, to form an insertion hole into which an implant can be inserted, the method including: applying force to a portion of a living body surface through which the puncture needle is to pass in a direction different from a direction in which the puncture needle punctures to establish a tensioned state; and puncturing the living body surface placed in the tensioned state by the puncture needle.

With the present disclosure, the medical tool includes the tensioning portion configured to apply, before a living body is punctured, force to a portion of the living body at which puncture by the puncture needle is to be started in a direction different from the direction in which the puncture needle punctures the living body tissue to establish a tensioned state. Therefore, puncture of the living body can be performed with relatively certainty and in a relative safe manner. Consequently, the puncture of the living body can be performed relatively easily. As a result, an implant can be embedded into the living body relatively easily, and the burden on the patient is relatively light and the safety of the patient is relatively high.

### BRIEF DESCRIPTION OF THE DRAWINGS

FIG. 1 is a perspective view depicting an example of an implant;

FIG. 2 is a perspective view depicting a puncture apparatus provided on a medical tool according to a first embodiment of the present disclosure;

FIG. 3 is a lateral view of the puncture apparatus depicted in FIG. 2;

FIG. 4 is a plan view depicting an operation member, which the puncture apparatus depicted in FIG. 2 includes;

FIGS. 5A and 5B depict a puncture member which the puncture apparatus depicted in FIG. 2 includes, wherein FIG. 5A is a perspective view and FIG. 5B is a sectional view taken along line VB-VB in FIG. 5A;

FIG. 6 is a cross sectional view of the puncture member depicted in FIGS. 5A and 5B;

FIGS. 7A to 7C depict a state maintaining portion which the puncture member depicted in FIGS. 5A and 5B includes, wherein FIG. 7A is a top plan view and FIGS. 7B and 7C are sectional views;

FIGS. 8A to 8C are partial enlarged views depicting the state maintaining portion which the puncture member depicted in FIGS. 5A and 5B has, wherein FIGS. 8A and 8B are plan views of modifications and FIG. 8C is a plan view of the present embodiment;

FIG. 9 is a plan view depicting a guide portion of a frame provided in the puncture apparatus depicted in FIG. 2;

FIG. 10 is a sectional view of the guide portion depicted in FIG. 9;

FIG. 11 is a plan view depicting a fixing portion of the frame, which the puncture apparatus depicted in FIG. 2 includes;

FIG. 12 is a lateral view of an insertion tool, which the puncture apparatus depicted in FIG. 2 includes;

FIGS. 13A and 13B are sectional views illustrating a function of a marker provided at a urethral-insertion portion in an inappropriate case and an appropriate case, respectively;

FIG. 14 is a sectional view illustrating the function of the marker provided at the urethral-insertion portion;

FIGS. 15A and 15B are a lateral view and a front view, respectively, depicting a positional relationship between the puncture member and the obturator foramen (pelvis);

FIGS. 16A and 16B are partial enlarged views of a vaginal-insertion tool which the insertion tool depicted in FIG. 12 includes, wherein FIG. 16A is a top plan view and FIG. 16B is a sectional view;

FIG. 17A is a sectional view depicting an example of a shape of a vaginal wall, and FIG. 17B is a sectional view depicting a vaginal-insertion portion in a state in which it is inserted in a vagina depicted in FIG. 17A;

FIG. 18 is a sectional view depicting the vaginal-insertion portion depicted in FIG. 12 in a state in which it is inserted in the vagina;

FIGS. 19A and 19B illustrate a maintaining portion and a displacement portion of the insertion tool depicted in FIG. 12, wherein FIG. 19A is a view depicting a locked state and FIG. 19B is a view depicting an unlocked state;

FIGS. 20A and 20B are views illustrating an operation procedure of the puncture apparatus depicted in FIG. 2;

FIGS. 21A and 21B are views illustrating another operation procedure of the puncture apparatus depicted in FIG. 2;

FIGS. 22A and 22B are views illustrating still another operation procedure of the puncture apparatus depicted in FIG. 2;

FIG. 23 is a lateral view illustrating a relationship between the puncture apparatus and the pelvis in a state illustrated in FIG. 22B;

FIG. 24 is a view illustrating an operation procedure of the puncture apparatus depicted in FIG. 2;

FIGS. 25A and 25B are views illustrating the operation procedure of the puncture apparatus depicted in FIG. 2;

FIGS. 26A and 26B are views depicting a procedure of a skin cutting operation;

FIGS. 27A and 27B are views illustrating the operation procedure of the puncture apparatus depicted in FIG. 2;

FIGS. 28A and 28B are views illustrating the operation procedure of the puncture apparatus depicted in FIG. 2;

FIG. 29 is a lateral view illustrating a relationship between the puncture apparatus and the pelvis in a state illustrated in FIG. 28A;

FIG. 30 is a sectional view depicting a posture of the puncture member with respect to a urethra in a state illustrated in FIG. 28B;

FIGS. 31A and 31B are views illustrating the operation procedure of the puncture apparatus depicted in FIG. 2;

FIG. 32 is a view illustrating the operation procedure of the puncture apparatus depicted in FIG. 2;

FIG. 33 is a plan view of a guide member provided on a medical tool according to a second embodiment of the present disclosure;

FIGS. 34A and 34B depict a guide member provided on a medical tool according to a third embodiment of the present disclosure, wherein FIG. 34A is a plan view of the guide member and FIG. 34B is a view as viewed in a direction indicated by an arrow mark B in FIG. 34A;

FIGS. 35A and 35B depict a guide member provided on a medical tool according to a fourth embodiment of the present disclosure, wherein FIG. 35A is a plan view of the guide member and FIG. 35B is a view as viewed in a direction indicated by an arrow mark C in FIG. 35A;

FIG. 36 is a view depicting a guide portion provided on a medical tool according to a fifth embodiment of the present disclosure;

FIG. 37 is a view depicting a guide portion provided on a medical tool according to a sixth embodiment of the present disclosure;

FIG. 38A is a view depicting a guide portion provided on a medical tool according to a seventh embodiment of the present disclosure, and FIG. 38B is a view as viewed in a direction indicated by an arrow mark D in FIG. 38A;

FIG. 39A is a view depicting a plate member of a guide portion provided on a medical tool according to an eighth embodiment of the present disclosure, and FIG. 39B is a sectional view of the plate member depicted in FIG. 39A and depicting a skin cutting operation;

FIG. 40A is a sectional view depicting a guide portion provided on a medical tool according to a ninth embodiment of the present disclosure, and FIG. 40B is a view depicting a procedure of a skin cutting operation;

FIG. 41A is a view depicting a guide portion provided on a medical tool according to a tenth embodiment of the present disclosure, and FIG. 41B is a view depicting a modification to a distal portion of the guide portion of FIG. 41A;

FIG. 42A is a view depicting a guide portion provided on a medical tool according to an eleventh embodiment of the present disclosure, and FIG. 42B is an enlarged view of an end portion of the guide portion of FIG. 42A;

FIG. 43 is a view depicting an end portion of a guide portion provided on a medical tool according to a twelfth embodiment of the present disclosure;

FIGS. 44A to 44C depict an end portion of a guide portion provided on a medical tool according to a thirteenth embodiment of the present disclosure, wherein FIG. 44A is a plan view, FIG. 44B is a view as viewed in the direction indicated by an arrow mark E in FIG. 44A, and FIG. 44C is a view illustrating a use state;

FIGS. 45A and 45B are views depicting a medical tool according to a fourteenth embodiment of the present disclosure and illustrating an operation procedure of the medical tool;

FIG. 46A is a view depicting a medical tool according to a fifteenth embodiment of the present disclosure, and FIG. 46B is a view as viewed in the direction indicated by an arrow mark F in FIG. 46A;

FIG. 47 is a view depicting a medical tool according to a sixteenth embodiment of the present disclosure;

FIG. 48A is a view depicting a medical tool according to a seventeenth embodiment of the present disclosure, and FIG. 48B is a view as viewed in the direction indicated by an arrow mark G in FIG. 48A;

FIG. 49A is a view depicting a medical tool according to an eighteenth embodiment of the present disclosure, and FIG. 49B is a view as viewed in the direction indicated by an arrow mark H in FIG. 49A;

FIG. 50 is a view depicting a medical tool according to a nineteenth embodiment of the present disclosure; and

FIG. 51 is a view depicting a medical tool according to a twentieth embodiment of the present disclosure.

### DETAILED DESCRIPTION

In the following, a medical tool and a puncture method of the present disclosure are described in detail in connection with preferred embodiments depicted in the accompanying drawings.

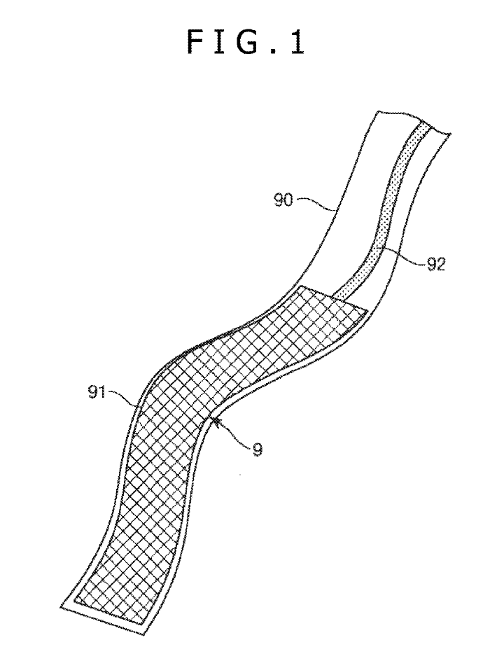

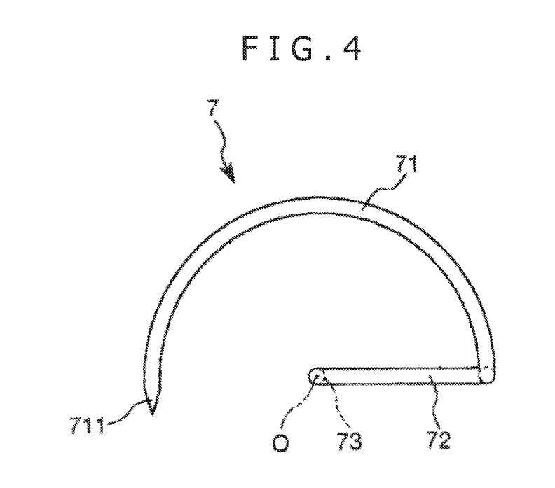

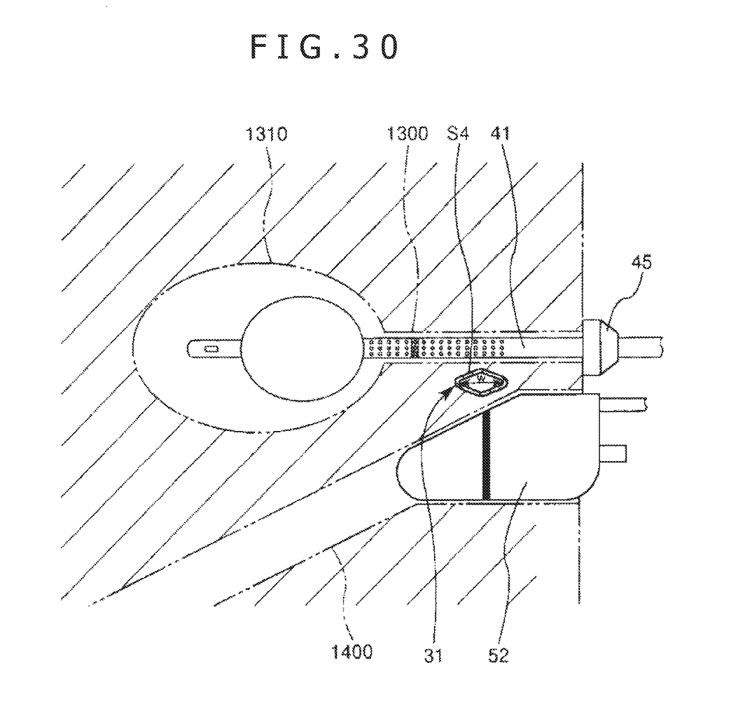

First, a medical tool and a puncture method according to a first embodiment are described below with reference to FIGS. 1 to 32.

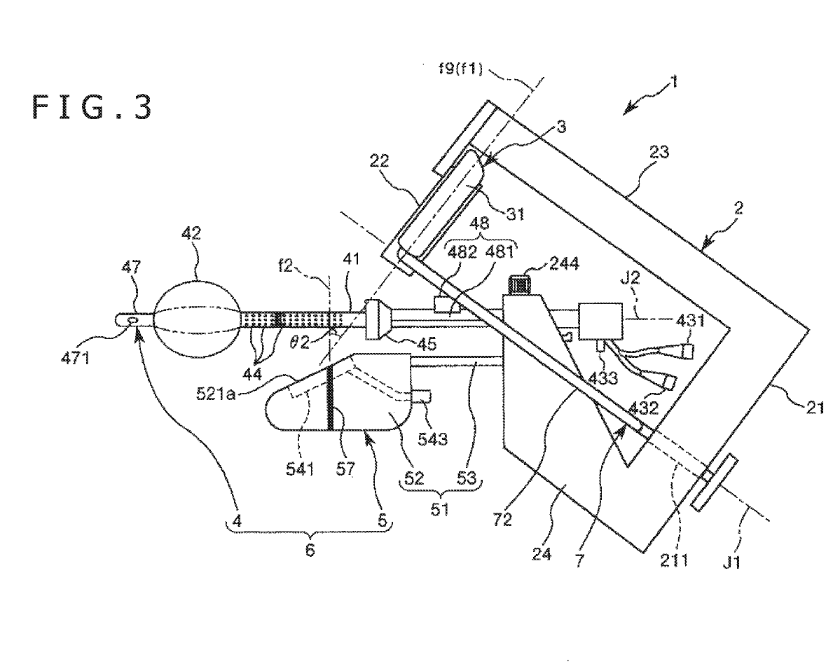

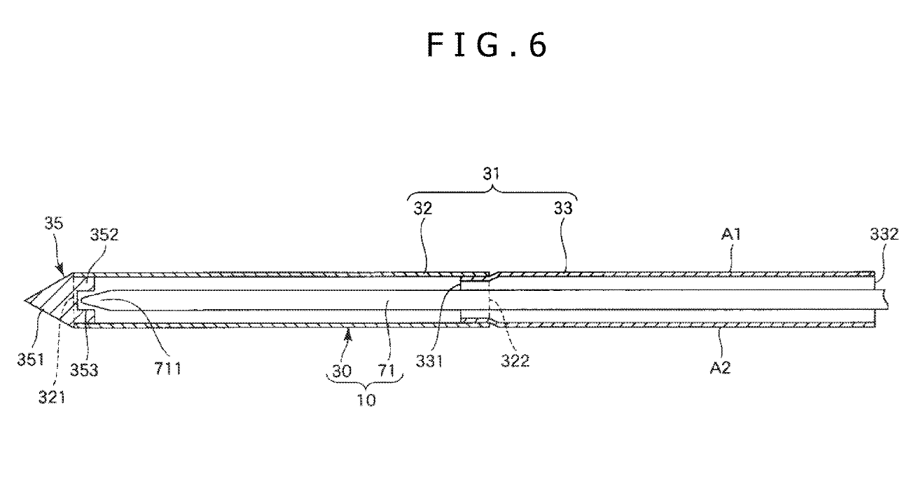

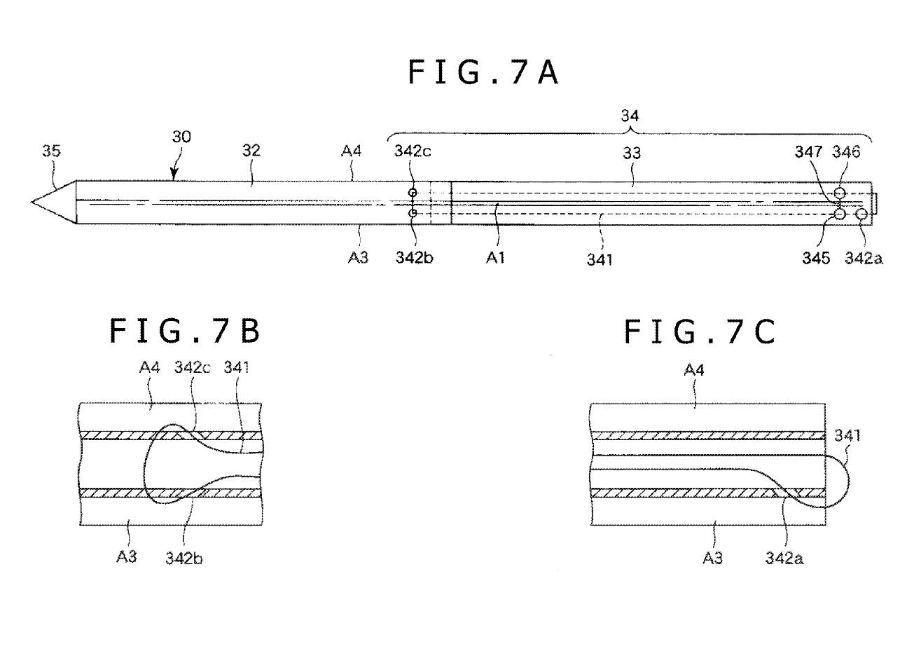

Note that, in the following description, the left side in FIG. 3 is referred to as “distal end,” the right side as “proximal end,” the upper side as “upper,” and the lower side as “lower” for the convenience of description. FIG. 3 depicts the puncture apparatus in a state in which it is not used as yet, and in the following description, this state is referred to as “initial state” for the convenience of description. Another state in which the puncture apparatus (insertion tool) depicted in FIG. 3 is mounted on a patient is referred to also as “mounted state” for the convenience of description. Furthermore, in FIGS. 6 and 7, the puncture member, which extends in an arc, is depicted in a linearly stretched state for the convenience of illustration.

A medical tool 100 of the present disclosure is a tool for embedding an implant 9 into a living body and can include a puncture apparatus 1, a skin cutting knife 11, and a guide member 8.

First, an example of an implant (living body tissue supporting indwelling article) 9 for being embedded in a living body by the puncture apparatus is described.

The implant 9 depicted in FIG. 1 is a tool, which can be embedded, in a living body for the treatment of female urinary incontinence. More particularly, the implant 9 is a tool for supporting a urethra, for example, a tool for supporting, when the urethra tends to move to the vaginal wall side, the urethra so as to restrict its movement in a direction in which the urethra is spaced away from the vaginal wall. As such, an implant 9 as just described, for example, an elongated article having flexibility can be used.

As depicted in FIG. 1, the implant 9 has an implant main body 91 and a belt 92 interlocked to one end of the implant main body 91. Note that, for example, a guide wire, a cord, or a string may be used in place of the belt 92. The implant main body 91 has a form of a net and has a belt-like general shape. The implant main body 91 can be configured from an article formed by crossing linear objects with each other into a braid, namely, a net-like braid. The linear objects may be those having a circular transverse sectional shape, or those having a flattened transverse sectional shape, namely, those of strip-like shape or the like.

The implant 9 having such a configuration as described above is accommodated in a packaging material 90 which is sterilized in an unused state. Consequently, contamination of the implant 9 can be prevented.

The constituent material of each of the implant main body 91, the belt 92, and the packaging material 90 is not restricted particularly, and various resin materials having biocompatibility such as polypropylene, polyester, nylon, etc. and their fibers can be used.

Although the implant 9 is described above, the implant 9 is not limited to the aforementioned net-like one only if it can demonstrate similar effects.

Further, a “pelvic treatment kit” can be configured from such an implant 9 as described above and a sheath 30 hereinafter described.

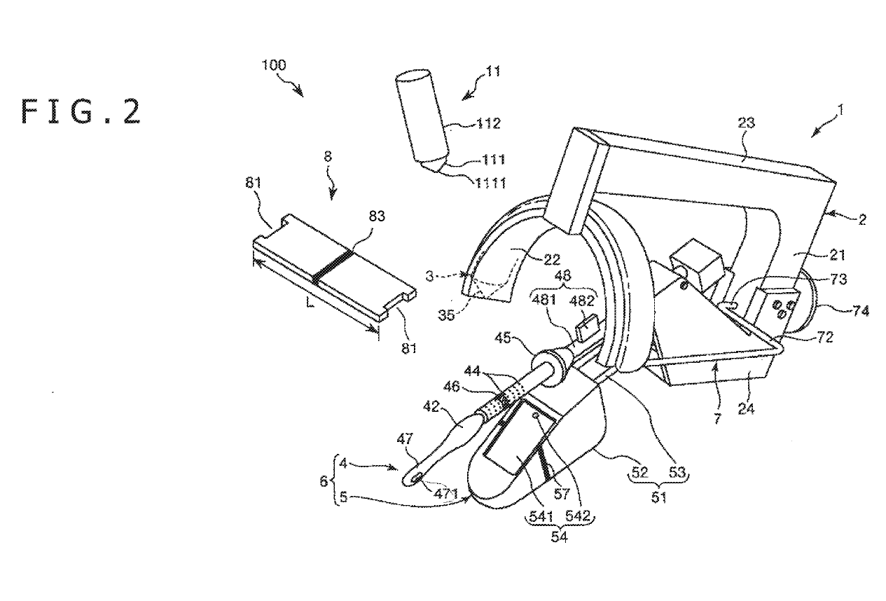

A puncture apparatus 1 depicted in FIG. 2 is an apparatus for embedding the aforementioned implant 9 into a living body.

As depicted in FIG. 2, the puncture apparatus 1 can include a frame (supporting portion) 2, an insertion tool 6, an operation member 7, and a puncture member (puncture needle) 3. The puncture member 3, the insertion tool 6, and the operation member 7 are supported on the frame 2, and the puncture member 3 is supported on the operation member 7. Further, the insertion tool 6 has a urethral-insertion tool 4 and a vaginal-insertion tool 5.

In the following, the components mentioned are described in order.

The operation member 7 is a member for supporting and operating the puncture member 3. As shown in FIGS. 2, 3, and 4, such an operation member 7 as just described can include an insertion portion 71, a shaft portion 73, and an interlock portion 72, which interlocks the insertion portion 71 and the shaft portion 73 to each other. The insertion portion 71, the interlock portion 72, and the shaft portion 73 may be formed integrally with one another, or, alternatively, at least one of them may be formed as a separate body from the others of them.

The insertion portion 71 is a portion to be inserted into the puncture member 3 and functions as a stylet for reinforcing the puncture member 3 from the inner side. By inserting the insertion portion 71 into the puncture member 3, the puncture member 3 is connected to the operation member 7, whereby an operation of the puncture member 3 by the operation member 7 (puncture of a living body) is permitted.

The insertion portion 71 has an arcuate shape corresponding to the shape of the puncture member 3. The center angle of the insertion portion 71 is set in accordance with the center angle of the puncture member 3. A distal portion 711 of the insertion portion 71 is tapering, by which the insertion of the insertion portion 71 into the puncture member 3 can be performed relatively smoothly.

Note that, while the transverse sectional shape of the insertion portion 71 in the present embodiment is a circular shape, the transverse sectional shape of the insertion portion 71 is not restricted to this, but may be, for example, an elliptic shape, a diamond shape rounded at the corners thereof, a rectangular shape (flat shape, plate shape) rounded at the corners thereof, or a spindle shape having an increased width (increased dimension) at a central portion with respect to the opposite end portions thereof. By applying such transverse sectional shapes, in a state in which the insertion portion 71 is inserted in the puncture member 3, rotation and so forth of the puncture member 3 with respect to the insertion portion 71 can be suppressed relatively effectively.

The shaft portion 73 extends along an axis J1 which intersects with the center O of the insertion portion 71 and is orthogonal to a plane f1 which can include the insertion portion 71. In accordance with an exemplary embodiment, the interlock portion 72 interlocks a proximal portion of the insertion portion 71 and a distal portion of the shaft portion 73. The interlock portion 72 has a substantially L shape bent at a substantially right angle at an intermediate portion thereof.

The operation member 7 having such a configuration as described above is configured so as to have a rigidity higher than that of the puncture member 3. The material constituting the operation member 7 is not restricted particularly and, for example, various metallic materials such as stainless steel, aluminum or aluminum alloys, and titanium or titanium alloys can be used. Note that, in order to suppress distortion and so forth of the puncture member 3, the interlock portion 72 and the shaft portion 73 may be formed thicker than the insertion portion 71 as occasion demands.

Further, a handle 74 can be fixed to a proximal portion of the shaft portion 73 such that the operation member 7 can be operated to turn around the axis J1 by rotationally operating the handle 74. Note that, for example, the interlock portion 72 may be grasped to operate the operation member 7 to rotate while the handle 74 is omitted.

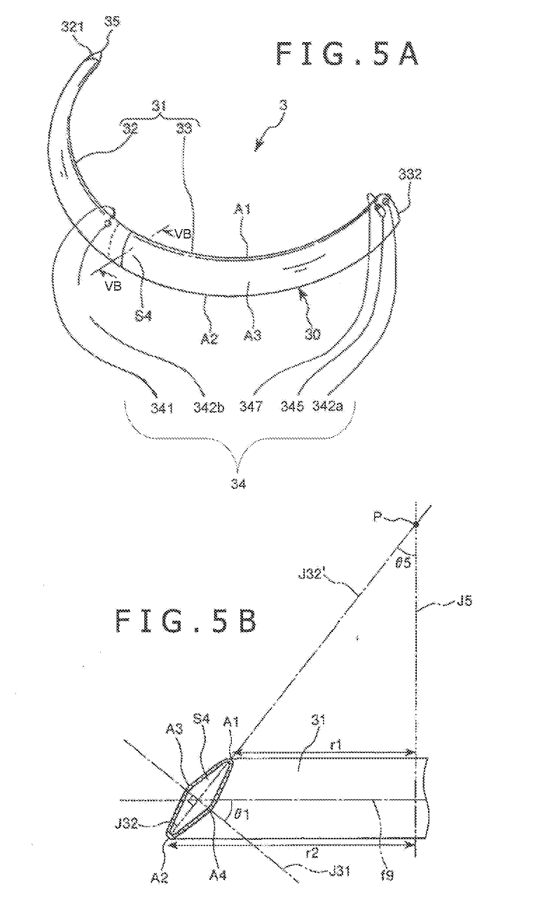

The puncture member 3 is a member for puncturing a living body. As shown in FIG. 5A, the puncture member 3 can include an elongated sheath (medical tube) 30, and a needle body 35 provided at the distal end of the sheath 30. The sheath 30 can include a tubular main body 31, and a state maintaining portion 34.

The main body 31 is configured from an elongated pipe member (tube) and is open at the distal end and the proximal end thereof. The inner side of the main body 31 functions as a space into which the insertion portion 71 is to be inserted and into which the implant 9 is to be inserted.

The main body 31 has a curved shape in which it is curved in an arc. Further, the main body 31 has a flattened transverse sectional shape having a minor axis J31 and a major axis J32 as depicted in FIG. 5B. By forming the main body 31 in a flattened shape, the implant main body 91 can be controlled to a desired posture in the main body 31.

Note that the flattened shape of the main body 31 is not restricted particularly and can be, for example, an elliptical shape, a sectional shape of a convex lens, a diamond shape rounded at the corners thereof, a rectangle (flattened shape) rounded at the corners thereof or a spindle shape having a width greater (diameter increased) at a central portion than at the opposite end portions thereof.

Meanwhile, the width (length in the major axis J32 direction) of the internal space of the main body 31 is designed so as to be substantially equal to the width of the implant main body 91. By the configuration, the frictional resistance between the implant 9 and the main body 31 is reduced and unnecessary force is not applied to the implant 9, and the implant main body 91 can be disposed into the main body 31 in a state in which it is developed sufficiently. However, the width of the internal space of the main body 31 may be smaller than the width of the implant main body 91. In this case, the width of the main body 31 is suppressed, and therefore, the puncture member 3 becomes minimally invasive.

Note that, in the following description, for the convenience of description, an end portion positioned on the inner side in the major axis J32 direction is referred to also as “inner circumferential portion A1,” an end portion positioned on the outer side in the major axis J32 direction be referred to also as “outer circumferential portion A2,” a face directed to the upper side is referred to also as “front face A3,” and a face directed to the lower side is referred to also as “back face A4,” as depicted in FIG. 5B.

Where, as depicted in FIG. 5B, a plane including the center point of the arc of a central portion S4 and the center point of a transverse sectional shape across the longitudinal direction of the main body 31 (plane including the center axis of the main body 31) is represented as plane f9 and the angle between the plane f9 and the minor axis J31 at the central portion S4 is represented as inclination angle θ1, the inclination angle θ1 preferably is an acute angle. Where the inclination angle θ1 is an acute angle, the implant main body 91 can be disposed substantially in parallel to the urethra as hereinafter described. Therefore, the urethra can be effectively supported.

Although the inclination angle θ1 is not restricted particularly only if it is an acute angle, it preferably is, for example, approximately 20 degrees to 60 degrees, more preferably is 30 degrees to 45 degrees, and most preferably is 35 degrees to 40 degrees. The aforementioned effects are further enhanced thereby.

Note that, although it is preferable for the inclination angle θ1 to satisfy the aforementioned numerical range over the overall region in the extending direction of the main body 31, only it is necessary for the inclination angle θ1 to satisfy the aforementioned numerical range at least at the central portion S4. Here, the “central portion S4” signifies a region including a region positioned between the urethra and the vagina at least in a state in which a living body is punctured by the puncture member 3 (a state in which the main body 31 is disposed inside the living body).

A marker may be provided at portions, which protrude to the outside of a living body in a state in which the main body 31 is disposed in the living body, of the opposite end portions of the main body 31 at positions spaced by an equal distance from the central portion S4. This makes it possible to confirm the position of the central portion S4 inside the living body by comparing the positions of both markers.

The configuration of the main body 31 can be described in the following manner. In particular, the main body 31 is formed such that the major axis J32 is inclined with respect to a center axis J5 of the arc and the center axis J5 and an extension line J32′ of the major axis J32 have an intersection P as depicted in FIG. 5B. In this case, the angle θ5 defined between the center axis J5 and the extension line J32′ is equal to the inclination angle θ1.

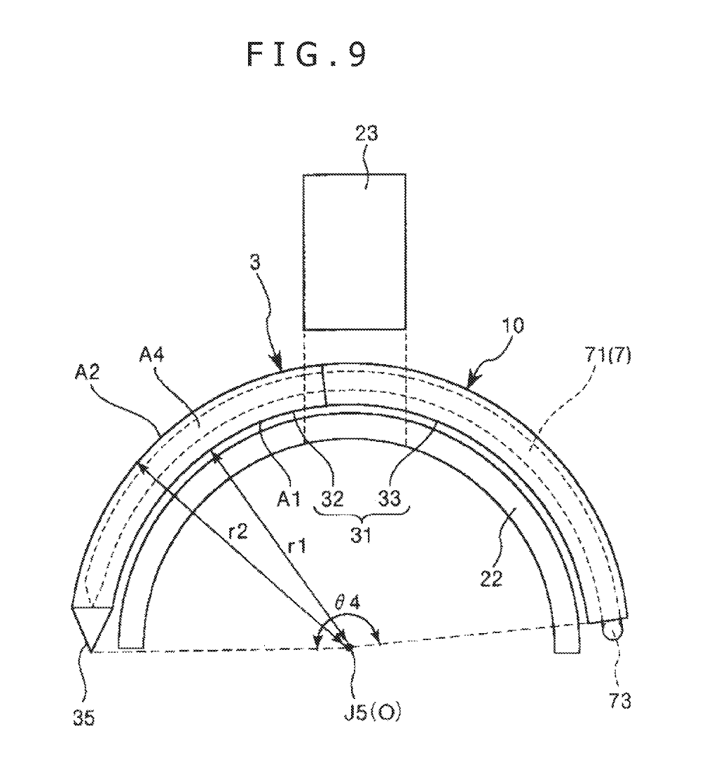

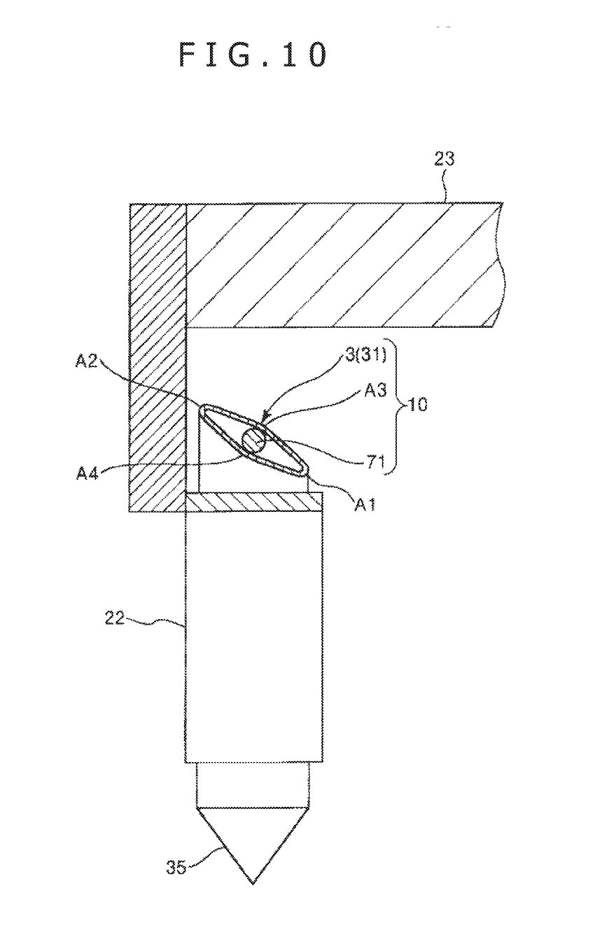

Alternatively, in a plan view as viewed in the direction of the center axis J5 of the main body 31 as depicted in FIG. 9, the main body 31 has the inner circumferential portion A1 positioned on an inner circumferential edge thereof and having a minimum radius of curvature r1 and the outer circumferential portion A2 positioned on an outer circumferential edge thereof and having a maximum radius of curvature r2, and, as depicted in FIGS. 5B and 10, the inner circumferential portion A1 and the outer circumferential portion A2 are positioned in a displaced relationship from each other in the direction of the center axis J5.

Such a main body 31 as described above is configured such that two divisional pieces are connected to each other such that the main body 31 can be divided halfway. In particular, the main body 31 has a distal end divisional piece 32 positioned on the needle body 35 side and a proximal end divisional piece 33 positioned on the proximal side of the distal end divisional piece 32. The distal end divisional piece 32 and the proximal end divisional piece 33 are connected for separation from each other. Further, in the main body 31, the distal end divisional piece 32 and the proximal end divisional piece 33 have lengths substantially equal to each other, and the boundary between them is positioned at the central portion S4.

As depicted in FIG. 6, the distal end divisional piece 32 has a tubular shape and has a distal side opening 321 and a proximal side opening 322. In addition, the proximal end divisional piece 33 has a tubular shape and has a distal side opening 331 and a proximal side opening 332. In accordance with an exemplary embodiment, a distal portion of the proximal end divisional piece 33 is inserted in a proximal portion of the distal end divisional piece 32, whereby the distal end divisional piece 32 and the proximal end divisional piece 33 are connected to each other. By inserting the proximal end divisional piece 33 in the distal end divisional piece 32, a step which may appear on the boundary between the divisional pieces 32 and 33 is less likely to be caught by the living body tissue at the time of puncture by the puncture member 3. Consequently, puncture of a living body by the puncture member 3 can be performed relatively smoothly. However, conversely to the present embodiment, a proximal portion of the distal end divisional piece 32 may be inserted in the proximal end divisional piece 33 to thereby connect the divisional pieces 32 and 33 together.

The state (connected state) in which the divisional pieces 32 and 33 are connected together is maintained by the state maintaining portion 34. As depicted in FIG. 7A, the state maintaining portion 34 has holes 342a, 342b and 342c, an endless string 341 fitted in the holes 342a, 342b and 342c, exposure holes 345 and 346 for exposing the string 341, and a slit 347 interconnecting the exposure holes 345 and 346

The hole 342a is provided at a proximal portion of the proximal end divisional piece 33 rather near to the inner circumferential portion A1 of the front face A3. In accordance with an exemplary embodiment, the holes 342b and 342c are provided in an opposing relationship to each other at positions of the front face A3 and the back face A4 of a proximal portion of the distal end divisional piece 32 rather near to the inner circumferential portion A1.

In accordance with an exemplary embodiment, the string 341 is disposed in the main body 31 and is routed outside the main body 31 between the hole 342b and the hole 342c and between the hole 342a and the proximal side opening 332. Consequently, the connection state of the divisional pieces 32 and 33 can be simply maintained. Further, exposure of the string 341 to the outside of the main body 31 can be suppressed, and the string 341 becomes less likely to be caught by a living body tissue. In addition, the overall length of the string 341 can be made as short as possible, therefore, the string 341 is less likely to be caught by the implant 9 at the time of inserting and passing the implant 9 into and through the main body 31. In accordance with an exemplar embodiment, for example, since the holes 342a, 342b and 342c are disposed rather near to the inner circumferential portion A1 as described hereinabove, the string 341 can also be disposed biasedly rather near to the inner circumferential portion A1. Therefore, the string 341 becomes less likely to be caught by the implant 9.

The string 341 can be obtained, for example, by preparing a string having ends, inserting one end of the string into the main body 31 via the proximal side opening 332, drawing out the same to the outside of the main body 31 through the hole 342b, inserting the same into the main body 31 via the hole 342c, drawing out the same to the outside of the main body 31 through the hole 342a and tying the same with the other end of the string in the proximity of the proximal side opening 332. Note, however, that the position of the knot is not limited.

Here, as shown in FIG. 7C, the axis of the hole 342a is inclined such that the outer side opening is positioned on the proximal side with respect to the inner side opening. In accordance with an exemplary embodiment, as shown in FIG. 7B, the axis of each of the holes 342b and 342c is inclined such that the outer side opening is positioned on the distal side with respect to the inner side opening. This makes it possible to allow each of the holes 342a, 342b and 342c to extend along the route of the string 341. Consequently, the string 341 is less likely to be caught by the holes 342a, 342b and 342c.

Note that the number and the disposition of holes through which the string 341 passes are not restricted particularly only if the connected state of the distal end divisional piece 32 and the proximal end divisional piece 33 can be maintained by the string 341. Further, the string 341 need not be in an endless state but may be in an ended state in which it has one end and the other end. For example, an ended string may be prepared and passed at one end thereof through the hole 342a and the proximal side opening 332 to form a loop while it passes at the other end thereof through the holes 342b and 342c to form a loop. Further, the string 341 can include a cord, a belt and so forth which can be used similarly to the string 341.

In accordance with an exemplary embodiment, the exposure holes 345 and 346 can be provided in an opposing relationship to each other at the front face A3 and the back face A4 of the proximal portion of the proximal end divisional piece 33. Further, the exposure holes 345 and 346 can be provided at locations at which they are exposed from the body surface when the main body 31 is disposed in a living body. Further, the exposure holes 345 and 346 are positioned on the route of the string 341 and the string 341 is exposed through the exposure holes 345 and 346. Further, the exposure holes 345 and 346 are tied with each other by the slit 347 provided in the inner circumferential portion A1 along a circumferential direction of the main body 31.

In such a state maintaining portion 34 as described above, if the string 341 is cut, then a state in which the distal end divisional piece 32 and the proximal end divisional piece 33 are separable from each other can be established. By the configuration, the distal end divisional piece 32 and the proximal end divisional piece 33 can be placed into a separable state through a relatively simple operation. Further, since the cutting of the string 341 is visually observable, it can be confirmed that the distal end divisional piece 32 and the proximal end divisional piece 33 are in a separable state.

By providing the exposure holes 345 and 346 and the slit 347 in such a manner as in the present embodiment, the string 341 can be cut. Describing by way of example, scissors are prepared, and one of blades thereof is fitted into the exposure holes 345 and 346 and the string 341 is positioned between the one and the other blades. Then, if the scissors are operated to close, then at least one of the pair of blades passes through the slit 347 until the pair of blades are overlapped with each other, and the string 341 is cut during the process.

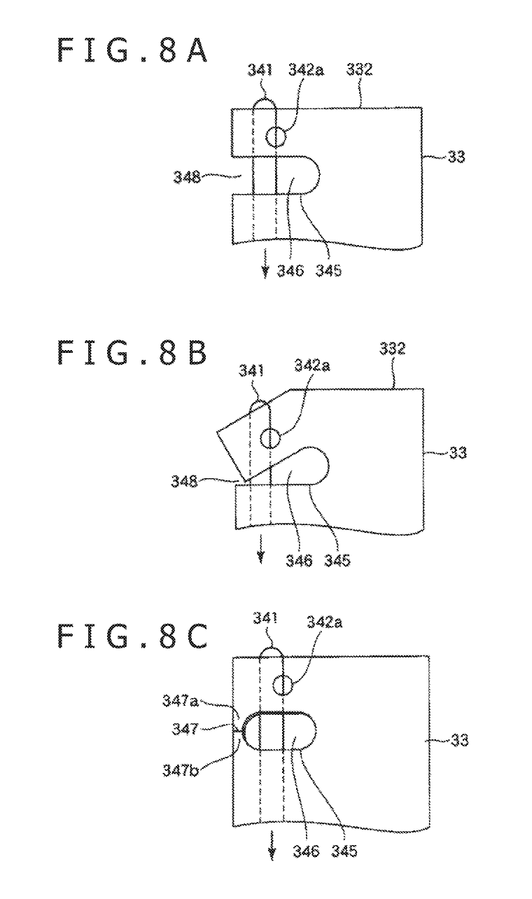

In this manner, in this embodiment, the slit 347 is used as a passing route of a blade. By this, cutting of the string 341 can be performed as described above, and deformation of the main body 31 by the tension on the string 341 can be prevented. For example, the passing route of a blade may be configured from a hole 348 in place of the slit 347 as shown in FIG. 8A. In this case, however, depending upon the hardness of the main body 31 or the like, the hole 348 may be crushed through buckling by the tension on the string 341 as depicted in FIG. 8B, resulting in deformation of the main body 31. In contrast, in the case of the slit 347, since portions 347a and 347b on the opposite sides of the slit 347 attach and are pressed against each other as depicted in FIG. 8C, such a deformation as described above will not occur. Consequently, the main body 31 is prevented from deformation.

The main body 31 described above has the needle body 35 at the distal end thereof. As depicted in FIG. 6, the needle body 35 can include a tapering needle tip 351, and a proximal portion 352 provided on the proximal side of the needle tip 351. The proximal portion 352 is inserted in the main body 31, whereby the needle body 35 is held on the main body 31 in a freely detachable manner. The proximal portion 352 is fitted in the main body 31 with force of such a degree that the needle body 35 can be prevented from being unintentionally removed from the main body 31.

In addition, the needle body 35 may be configured integrally with the main body 31, and in this case, only it is necessary for the needle body 35 to be cut away from the main body 31 by scissors or the like.

Further, an engaging portion 353 for engaging with the distal portion 711 of the insertion portion 71 is provided at the proximal portion 352. The engaging portion 353 is configured from a recess, and in an insertion state in which the insertion portion 71 is inserted in the puncture member 3, the distal portion 711 is positioned in the engaging portion 353. Consequently, displacement of the puncture member 3 with respect to the insertion portion 71, more particularly, rotation of the puncture member 3 with respect to the insertion portion 71, displacement of the puncture member 3 in a widthwise direction with respect to the insertion portion 71 or the like, is inhibited, and puncture of the puncture member 3 into a living body can be performed with increased smoothness.

The puncture member 3 has been described above. A center angle θ4 (refer to FIG. 9) of the puncture member 3 is not restricted particularly and is appropriately set in accordance with various conditions. In accordance with an exemplary embodiment, however, the center angle θ4 is set such that the needle body 35 can enter the body of a patient via one of the inguinal regions, pass between the urethra and the vagina and leave the body via the other inguinal region. In particular, the center angle θ4 preferably is, for example, approximately 150 degrees to 270 degrees, more preferably is approximately 170 degrees to 250 degrees, and most preferably is approximately 190 degrees to 230 degrees.

The constituent materials of the main body 31 and the needle body 35 preferably are such rigid materials as to maintain the shape and the internal space of them in a state where the puncture member 3 is inserted in a living body. As such rigid materials, for example, various resin materials such as polyethylene, polyimide, polyamide, polyester elastomer or polypropylene or various metal materials such as stainless steel, aluminum or aluminum alloys, titanium or titanium alloys can be used.

Note that, where the main body 31 and the needle body 35 are configured not only by adopting rigid materials but also by adopting other materials than rigid materials, the wall may be reinforced with a reinforcement member. For example, a braid of high strength may be embedded in the wall to maintain the shape and the internal space in the state in which the puncture member 3 is inserted in a body. Another example of the reinforcement member is a spiral body, which is embedded in the wall of the main body 31, by which flexibility can be provided while the internal space is maintained to such an extent that an inserted article can be slidably moved therein.

The main body 31 preferably is optically transparent so that the inside thereof can be visually observed from the outside, which helps make it possible, for example, to confirm from the outside whether the distal portion 711 of the insertion portion 71 inserted in the inside of the main body 31 is in engagement with the engaging portion 353, whether the string 341 has not been cut, and so on.

In the puncture apparatus 1, such a puncture member 3 (main body 31) as described above and the insertion portion 71 which is inserted in the main body 31 cooperatively configure a medical tube assembly 10, and use of the puncture apparatus 1 is started in a state in which the members configure the medical tube assembly 10.

The frame 2 holds the operation member 7, on which the puncture member 3 is mounted, for rotary movement and has the insertion tool 6 fixed thereto in a freely detachable manner. Such a frame 2 as just described has a function of determining a puncture route by the needle body 35 when the puncture member 3 punctures the living body tissue. In particular, the frame 2 determines the positional relationship of the puncture member 3, urethral-insertion tool 4 and vaginal-insertion tool 5 such that, when the puncture member 3 punctures the living body tissue, the needle body 35 passes between the urethral-insertion tool 4 and the vaginal-insertion tool 5 without colliding with any of the insertion tools.

As depicted in FIGS. 2 and 3, the frame 2 can include a bearing portion 21 for bearing the shaft portion 73 of the operation member 7, a guide portion 22 for guiding the puncture member 3, an interlock portion 23 for interlocking the bearing portion 21 and the guide portion 22 to each other, and a fixing portion 24 to which the insertion tool 6 is to be fixed.

In accordance with an exemplary embodiment, the bearing portion 21 is positioned on the proximal side of the puncture apparatus 1 and extends in a direction substantially orthogonal to the axis J1. The bearing portion 21 has a through-hole 211 formed on the axis J1 thereof, and the shaft portion 73 is inserted for rotary movement in the through-hole 211. As a result, the operation member 7 is supported on the frame 2 so as to move rotationally around the axis J1.

The guide portion 22 is positioned on the distal side of the puncture apparatus 1 and disposed in an opposing relationship to the bearing portion 21. As depicted in FIG. 9, the guide portion 22 is positioned on the inner side of the puncture member 3 and is configured such that it has an arcuate shape extending along the puncture member 3 and supports the puncture member 3 from the inner side. As depicted in FIG. 10, the puncture member 3 is disposed on the guide portion 22 such that the back face A4 is positioned on the distal side and the front face A3 is positioned on the proximal side.

The interlock portion 23 interlocks the bearing portion 21 and the guide portion 22 to each other. The interlock portion 23 has a form of a bar extending substantially in parallel to the axis J1. The interlock portion 23 can also function as a grasping portion. In accordance with an exemplary embodiment, an operator can use the puncture apparatus 1 in a stable state by grasping the interlock portion 23.

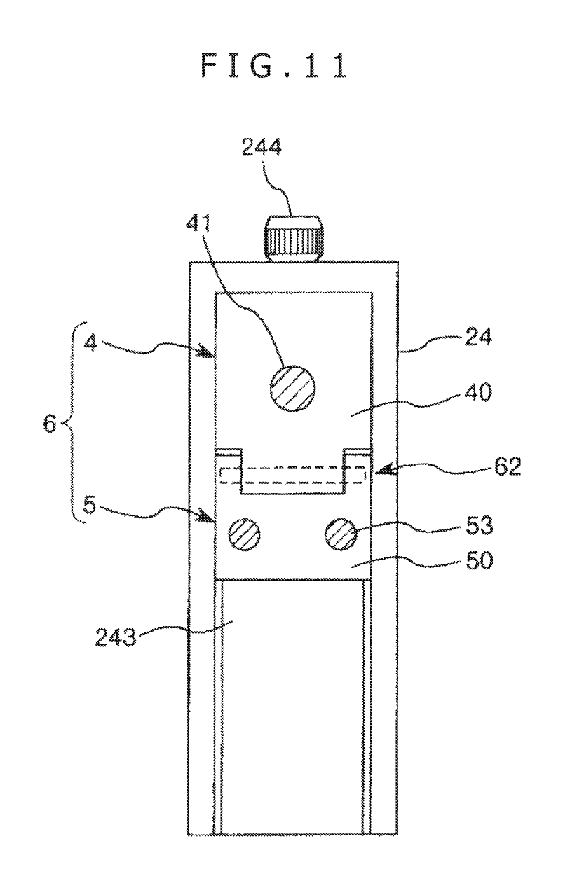

The fixing portion 24 is disposed in an opposing relationship to the interlock portion 23 with the axis J1 interposed therebetween. The fixing portion 24 has a recess 243 in which to fit supporting portions 40 and 50 hereinafter described of the insertion tool 6, and a male screw 244 as depicted in FIG. 11. With such a fixing portion 24 as just described, the insertion tool 6 can be fixed to the fixing portion 24 by fitting the supporting portions 40 and 50 into the recess 243 and then tightening the male screw 244 into the supporting portion 40.

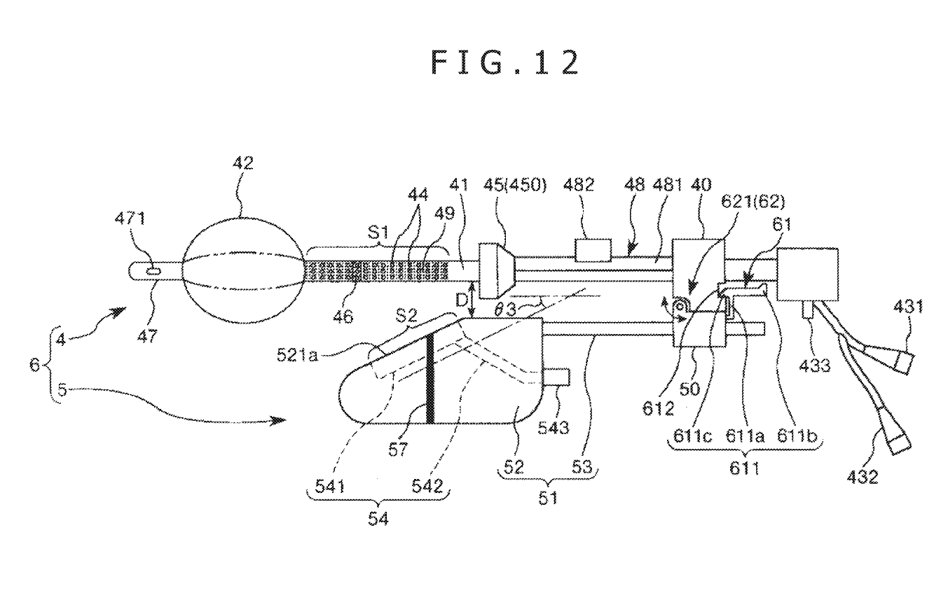

As depicted in FIG. 12, the insertion tool 6 has the urethral-insertion tool 4 and the vaginal-insertion tool 5.

The urethral-insertion tool 4 has an elongated urethral-insertion portion 41 for being inserted up to the middle thereof into the urethra, and the supporting portion (urethral-insertion portion supporting portion) 40 for supporting the urethral-insertion portion 41. As regards the constituent materials of the urethral-insertion portion 41 and the supporting portion 40, there is no particular restriction, and various metal materials such as, for example, stainless steel, aluminum or aluminum alloys, or titanium or titanium alloys or various resin materials can be used.

Further, the length of the urethral-insertion portion 41 (portion on the distal side from the supporting portion 40) is not restricted particularly but is suitably set depending upon the length of the urethra, the shape of the bladder and so forth of the patient. Since the length of the urethra of most women is approximately 30 mm to 50 mm, the length of the urethral-insertion portion 41 is preferably set to, for example, approximately 50 mm to 100 mm with reference to the length of the urethra.

The urethral-insertion portion 41 has a straight tube shape. At a distal portion of such a urethral-insertion portion 41 as just described, a balloon (attachment portion) 42 which is an expandable/contractible expansion body, and a urine drainage portion 47.

The balloon 42 is disposed such that it is positioned in the bladder when the urethral-insertion portion 41 is inserted in the urethra. The balloon 42 extends in the urethral-insertion portion 41 and is connected to a balloon port 431 provided at a proximal portion of the urethral-insertion portion 41. A balloon expansion tool such as a syringe can be connected to the balloon port 431, and if a working fluid (liquid such as physiological saline solution, or a gas or the like) is supplied into the balloon 42 from the balloon expansion tool, then the balloon 42 is expanded. In contrast, if the working fluid is extracted from the balloon 42 by the balloon expansion tool, then the balloon 42 is contracted. Note that in FIG. 12, the state in which the balloon 42 is contracted is indicated by an alternate long and short dashes line and the state in which the balloon 42 is expanded is indicated by a solid line.

The urine drainage portion 47 is used to drain the urine in the bladder in a state in which the urethral-insertion portion 41 is inserted in the urethra. A urine drain hole 471 is provided in the urine drainage portion 47 such that it extends between the inside and the outside of the urine drainage portion 47. Further, the urine drain hole 471 extends through the inside of the urethral-insertion portion 41 and is connected to a urine drain port 432 provided at a proximal portion of the urethral-insertion portion 41. Therefore, the urine introduced from the urine drain hole 471 can be drained from the urine drain port 432.

The balloon 42 and the urine drainage portion 47 can be configured, for example, from a double lumen.

Further, in accordance with an exemplary embodiment, a plurality of suction holes 44 can be located in the urethral-insertion portion 41 on a proximal side of the balloon, and more particularly, for example, the plurality of suction holes 44 can be located at an mid-portion of the urethral-insertion portion 41. The plurality of suction holes 44 can be formed as a suction portion for absorbing a urethral wall to the urethral-insertion portion 41.

The plurality of suction holes 44 can be disposed over an overall area in a circumferential direction of the urethral-insertion portion 41. The suction holes 44 are connected to a suction port 433 provided on the supporting portion 40 through the inside of the urethral-insertion portion 41. A suction device such as a pump can be connected to the suction port 433, and if the suction device is rendered operative in a state in which the urethral-insertion portion 41 is inserted in the urethra, then the urethral wall can be absorbed and fixed to the suction holes 44. In accordance with an exemplary embodiment, for example, by providing the plurality of suction holes 44 over an overall area in a circumferential direction of the urethral-insertion portion 41 as in the present embodiment, the urethral wall can be absorbed and fixed over a wide range thereof to the urethral-insertion portion 41.

Note that the number of the suction holes 44 is not restricted particularly, and for example, only one suction hole may be provided. Further, the layout of the suction holes 44 is not restricted particularly, and, for example, the suction holes 44 may be formed in only part of the urethral-insertion portion 41 in the circumferential direction.

If the urethral-insertion portion 41 is pushed in to the inside of the body (to the distal side of the urethral-insertion portion 41) in the state in which the urethral wall is absorbed and fixed to the urethral-insertion portion 41 in this manner, then the urethra and the bladder are pushed in to the inside of the body together with this. Consequently, the bladder can be displaced to a position at which it does not overlap with a puncture route of the puncture member 3. Therefore, a greater puncture route can be secured for the puncture member 3, and puncture of the puncture member 3 can be performed relatively accurately and safely.

Further, a visually observable marker (detection portion) 46 can be provided, for example, at an approximate mid-portion (or halfway) between the ends of the urethral-insertion portion 41 on the proximal side with respect to the balloon 42. This marker 46 can be used to detect the distance between the bladder and the urethral orifice, or in other words, the length of the urethra.

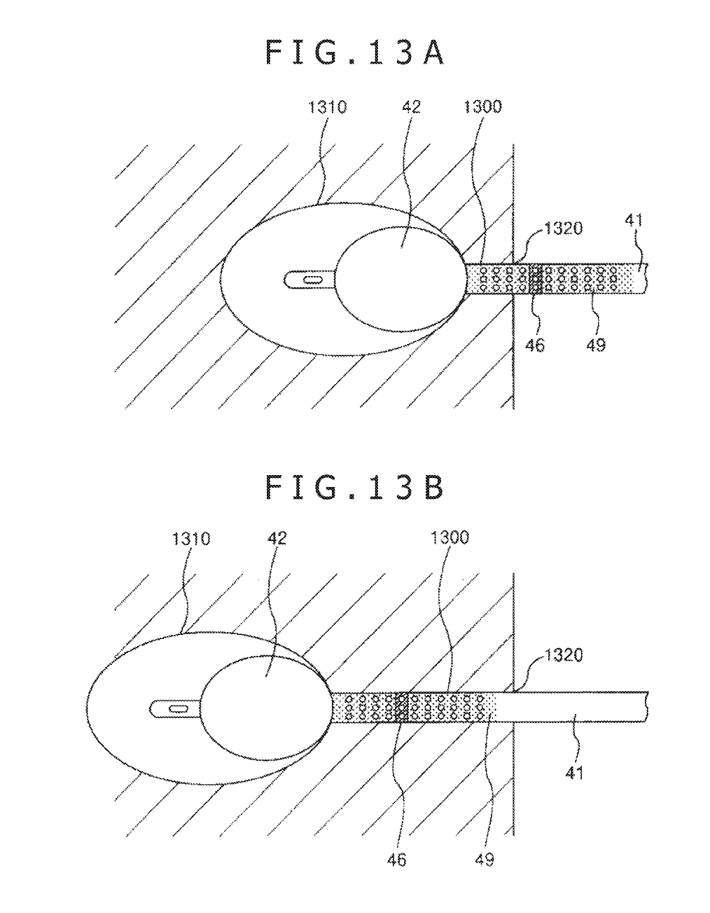

In accordance with an exemplary embodiment, for example, where the separated distance between a bladder 1310 and a urethral orifice 1320 is smaller than a predetermined distance as depicted in FIG. 13A, in a state in which the urethral-insertion portion 41 is inserted in a urethra 1300 and the balloon 42 is expanded until it is attached to the bladder neck, the marker 46 can be exposed from the urethral orifice 1320. In contrast, if the separated distance between the bladder 1310 and the urethral orifice 1320 is greater than the predetermined distance as depicted in FIG. 13B, then in the state in which the urethral-insertion portion 41 is inserted in the urethra 1300 and the balloon 42 is expanded until it is attached to the bladder neck, the marker 46 is positioned in the urethra 1300 and is not exposed from the urethral orifice 1320.

If the marker 46 is exposed from the urethral orifice 1320 as depicted in FIG. 13A, then it can be estimated that the distance between the bladder 1310 and the urethral orifice 1320 is relatively short and a sufficient space for allowing puncture by the puncture member 3 does not exist between them. Therefore, in this case, it is decided that puncture by the puncture member 3 cannot be performed.

In contrast, if the marker 46 is not exposed from the urethral orifice 1320 as depicted in FIG. 13B, then it is estimated that the distance between the bladder 1310 and the urethral orifice 1320 is sufficiently great and a sufficient space for allowing puncture by the puncture member 3 exists between them. Therefore, in this case, it is decided that puncture by the puncture member 3 can be performed.

In this manner, by providing the marker 46, it is possible to decide simply whether or not puncture by the puncture member 3 through the puncture apparatus 1 is possible, or in other words, whether or not the puncture apparatus 1 can be applied to the patient. In accordance with an exemplary embodiment, for example, by disposing the balloon 42 and causing the balloon 42 to attach to the bladder neck, the separated distance between the bladder 1310 and the urethral orifice 1320 can be detected relatively accurately, and the aforementioned decision can be performed with enhanced accuracy.

The configuration of the marker 46 is not restricted particularly only if the operator can visually observe the marker 46 from the outside. For example, the marker 46 may be configured from a portion colored in a different color from those of surrounding elements (distal side and proximal side with respect to the marker 46) or may be configured from a recess or a projection.

Alternatively, the marker 46 may be graduations to which distances from the proximal end of the balloon 42 are inscribed. In this case, since whether or not puncture by the puncture member 3 is possible cannot be decided depending upon whether or not the marker 46 is exposed from the urethral orifice 1320 as in the present embodiment, the length of the urethra 1300 may be measured by the graduations to decide whether or not puncture by the puncture member 3 is possible depending upon the length.

Further, as depicted in FIGS. 12, 13A and 13B, a sliding resistance amplification portion 49 for contacting with the urethral wall (tissue in the urethra) to raise the sliding resistance with respect to the urethra can be provided at a distal portion of the urethral-insertion portion 41. Although the bladder 1310 is displaced sufficiently to a position at which it does not overlap with the puncture route of the puncture member 3 by absorbing and fixing the urethral wall to the urethral-insertion portion 41 during a procedure in which the puncture apparatus 1 is used and pushing in the urethral-insertion portion 41 to the inside of the body as described above, if the absorption of the urethral wall by the suction holes 44 thereupon is not sufficient, then the urethral wall may be come off from the suction holes 44, resulting in failure to push in the bladder 1310 sufficiently. Therefore, by providing the sliding resistance amplification portion 49 on the urethral-insertion portion 41 so that the urethral-insertion portion 41 is less likely to slip with respect to the urethral wall as in the present embodiment, even in a case in which absorption by the suction holes 44 is not sufficient, the bladder 1310 can be pushed in to the inside of the body, or even in another case in which absorption by the suction holes 44 is sufficient, the bladder 1310 can be pushed in to the inside of the body with a higher degree of certainty by a synergetic effect. Therefore, puncture by the puncture member 3 can be performed relatively safely.

The configuration of the sliding resistance amplification portion 49 is not restricted particularly if the aforementioned effect can be achieved. In the present embodiment, the sliding resistance amplification portion 49 is configured from fine recesses/projections (rough surface) formed on the surface of the urethral-insertion portion 41. Consequently, the configuration of the sliding resistance amplification portion 49 can be simplified. Note that the aforementioned recesses/projections signify that only it is necessary for the configuration of the sliding resistance amplification portion 49 to have at least one of recesses and projections.

Further, the sliding resistance amplification portion 49 is provided over an overall area in a circumferential direction of the urethral-insertion portion 41. Consequently, since the area of the sliding resistance amplification portion 49 can be made greater, the sliding resistance with respect to the urethra 1300 can be increased more. Note that the sliding resistance amplification portion 49 can be formed by embossing, sandblasting, laser processing or the like.

Further, the sliding resistance amplification portion 49 is provided in an overlapping relationship with a region S1 in which the suction holes 44 are formed. In other words, the suction holes 44 are formed at the sliding resistance amplification portion 49. If the urethral wall is absorbed by the suction holes 44, then the urethral wall bites into and can be caught by the suction holes 44. Since such an anchor effect as just described is demonstrated in the sliding resistance amplification portion 49, the sliding resistance of the urethral-insertion portion 41 with respect to the urethra can be increased more. Further, since the sliding resistance amplification portion 49 is provided in an overlapping relationship with the region S1, the sliding resistance amplification portion 49 and the region S1 can be disposed widely, and therefore, higher effects can be demonstrated by both of them.

Further, a tubular marker (positioning portion) 45 can be provided for sliding movement on the urethral-insertion portion 41. Further, a restriction portion 48 can be mounted on the urethral-insertion portion 41 between the marker 45 and the supporting portion 40 such that, when the marker 45 is attached to the restriction portion 48, further sliding movement of the marker 45 toward the proximal side is restricted. The restriction portion 48 can be removed simply from the urethral-insertion portion 41. If the restriction portion 48 is removed from the urethral-insertion portion 41, then the marker 45 can be slidably moved further to the proximal side.

The configuration of the restriction portion 48 is not restricted particularly only if the aforementioned effect can be achieved. In the present embodiment, the restriction portion 48 has a main body 481 having a substantially C-like semi-cylindrical shape as a transverse sectional shape thereof, and a knob 482 provided on the main body 481. The main body 481 is mounted on the urethral-insertion portion 41, for example, in a state in which it has a diameter increased a little so that it may not be removed from the urethral-insertion portion 41 by its own weight or may not slidably move with respect to the urethral-insertion portion 41.

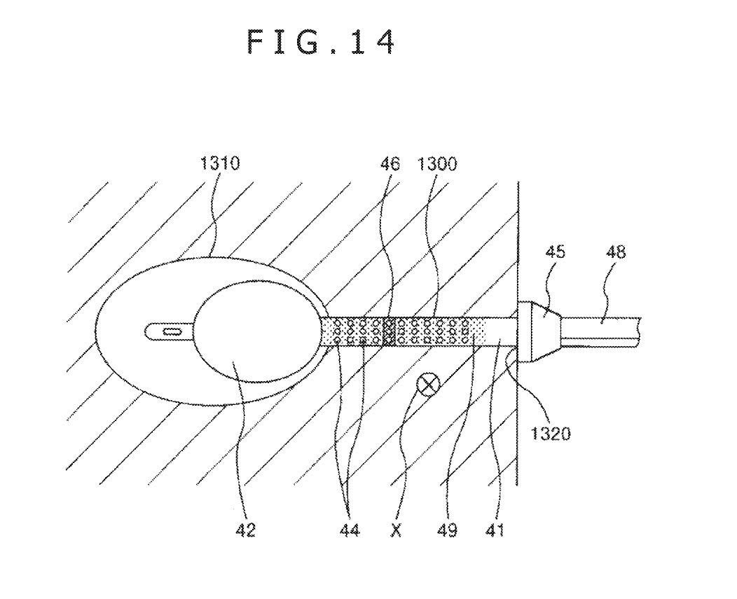

The marker 45 is positioned such that, in a state in which it is attached to the restriction portion 48 and is restricted from slidably moving further to the proximal side (state illustrated in FIG. 12), it has a predetermined relative positional relationship with the puncture route of the puncture member 3. Therefore, if the urethral-insertion portion 41 is inserted into the urethra 1300 and the marker 45 is positioned at the urethral orifice 1320 (or in the proximity of the urethral orifice 1320) as depicted in FIG. 14, then a puncture route X of the puncture member 3 can be positioned with respect to the urethral orifice 1320 (or in the proximity of the urethral orifice 1320). By positioning the puncture route X with respect to the urethral orifice 1320 (or in the proximity of the urethral orifice 1320) in this manner, the accuracy in positioning is enhanced and accurate puncture by the puncture member 3 can be performed in comparison with an alternative case in which the puncture route X is positioned, for example, with respect to the bladder 1310. Here, the “predetermined relative positional relationship” can be regarded as such a positional relationship that, for example, in a state in which the marker 45 is positioned at the urethral orifice 1320, the puncture route X of the puncture member 3 does not overlap with the bladder 1310.

Further, since the marker 45 has an outer diameter greater than that of the urethral-insertion portion 41 and projects to the outer side of the urethral-insertion portion 41, it cannot be inserted into the urethra 1300. Therefore, if the urethral-insertion portion 41 is inserted into the urethra 1300, then the marker 45 is attached, halfway of the insertion, to the urethral orifice 1320 (or the tissue around the urethral orifice 1320: this similarly applies also to the following description) and the marker 45 can be positioned at the urethral orifice 1320 naturally. By utilizing a physical catch in this manner, the marker 45 can be positioned at the urethral orifice 1320 simply and with relative certainty. Further, when the marker 45 is attached to the urethral orifice 1320, further insertion of the urethral-insertion portion 41 into the urethra 1300 is restricted, and therefore, excessive insertion of the urethral-insertion portion 41 into the urethra 1300 can be prevented and the safety of the puncture apparatus 1 can be enhanced.

Such a marker 45 as described above has, in addition to such a function of positioning the puncture route of the puncture member 3 with respect to the urethral orifice 1320 as described above, another function as an inflow reduction portion 450 for suppressing inflow of air into the urethra 1300 through the urethral orifice 1320 in a state in which the urethral-insertion portion 41 is inserted in the urethra 1300. Describing more particularly, in a state in which the urethral-insertion portion 41 is inserted in the urethra 1300 and the marker 45 is attached to the urethral orifice 1320 as depicted in FIG. 14, the urethral orifice 1320 is covered with and closed up by the marker 45. Therefore, it can be difficult for air to flow into the urethra 1300 through the urethral orifice 1320, and inflow of air into the urethra 1300 (suction holes 44) through the urethral orifice 1320 can be suppressed. As a result, absorption of the urethral wall by the suction holes 44 can be performed with a higher degree of certainty and effectiveness.

Since the marker 45 serves also as the inflow reduction portion 450 in this manner, the configuration of the urethral-insertion tool 4 is simplified and also an operation of the urethral-insertion tool 4 is simplified. Further, the number of component parts can be reduced, which contributes to a reduction in cost.

Now, inclination between the urethral-insertion portion 41 and the puncture member 3 is described. As depicted in FIG. 3, although the inclination angle (in other words, the angle defined by the axes J1 and J2) 82 of the plane f9 (plane f1) with respect to the plane f2 orthogonal to the axis J2 of the urethral-insertion portion 41 is not restricted particularly, it preferably is, for example, approximately 20 degrees to 60 degrees, more preferably is approximately 30 degrees to 45 degrees, and most preferably is approximately 35 degrees to 40 degrees. By this, puncture by the puncture member 3 can be performed relatively easily. Further, the puncture distance by the puncture member 3 can be made shorter.

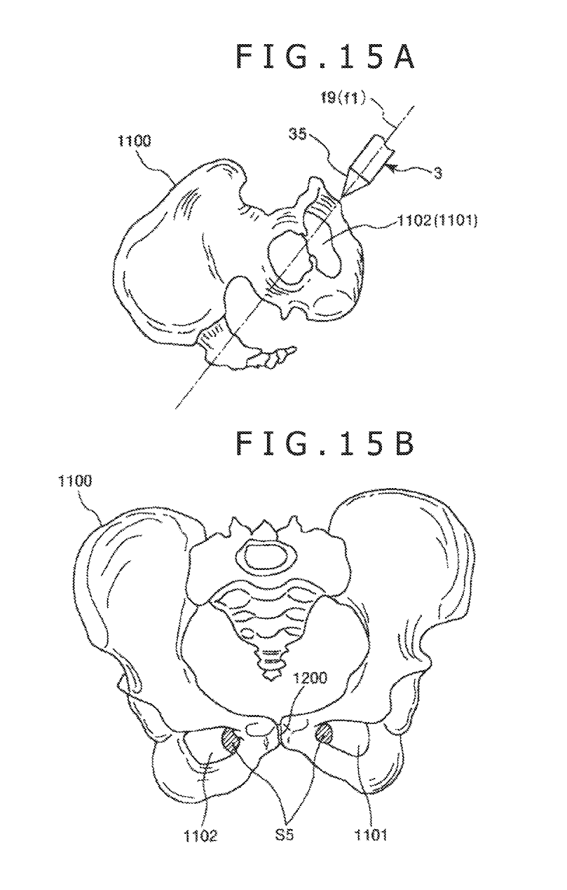

Describing more particularly, by setting the inclination angle θ2 within the aforesaid range, the puncture member 3 can capture left and right obturator foramens 1101 and 1102 of a pelvis 1100 widely on a plane as depicted in FIG. 15A, and a wide puncture space can be secure for the puncture member 3. In other words, in a state in which the patient assumes a predetermined posture (lithotomy position), the puncture member 3 can puncture in a direction comparatively near to the vertical direction relative to the obturator foramens 1101 and 1102. Therefore, the puncture by the puncture member 3 can be performed relatively easily.

In addition, by causing the puncture member 3 to puncture in a direction comparatively near to the vertical direction relative to the obturator foramens 1101 and 1102, the puncture member 3 passes a shallow portion of the tissue. Therefore, the needle body 35 of the puncture member 3 can pass between the left and right obturator foramens 1101 and 1102 taking a shorter course. Therefore, as depicted in FIG. 15B, the puncture member 3 can pass those zones in the obturator foramens 1101 and 1102 rather near to a pubic symphysis 1200, preferably, safety zones S5. Since the safety zones S5 are regions, which include comparatively small number of nerves and blood vessels to which damage should be avoided, the puncture member 3 passes the safety zones S5 so that the puncture can be performed by the puncture member 3 safely. Therefore, a less invasive procedure can be realized, and the burden on the patient can be suppressed to a relatively low level.

In this manner, by setting the inclination angle θ2 within the aforesaid range, the puncture of the patient by the puncture member 3 can be performed appropriately. Further, the puncture at the aforementioned angle makes it easier to aim at the tissue between a middle urethra (which refers to a middle portion of the urethra in the longitudinal direction) and the vagina. Here, since the tissue between the middle urethra and the vagina is a position suitable as a region into which the implant 9 is to be embedded to carry out treatment for urinary incontinence, an effective treatment can be performed by targeting the tissue between the middle urethra and the vagina.

In contrast, where the inclination angle θ2 is smaller than the aforesaid lower limit or greater than the aforesaid upper limit, depending upon individual differences of the patient, the posture of the patient during the procedure or the like, it sometimes can occur that the puncture member 3 cannot capture the obturator foramens 1101 and 1102 widely on a plane or the puncture route cannot be made sufficiently short.

The configuration of the urethral-insertion tool 4 has been described above. In such a urethral-insertion tool 4 as described above, the urethral-insertion portion 41 may be configured so as not to be able to slidably move with respect to the supporting portion 40 or so as to be able to slidably move with respect to the supporting portion 40. Where the urethral-insertion portion 41 is configured for sliding movement, the urethral-insertion portion 41 may be configured such that, for example, if a screw (not depicted) provided on the supporting portion 40 is loosened, then the urethral-insertion portion 41 is permitted to slidably move with respect to the supporting portion 40, but if the screw is tightened, then the urethral-insertion portion 41 is placed into a state in which it is fixed to the supporting portion 40. With the configuration just described, since the length of the urethral-insertion portion 41 can be adjusted, the urethral-insertion tool 4 is improved further in convenience in use. Note that this similarly applies also to the vaginal-insertion tool 5 hereinafter described.

Further, while, in the puncture apparatus 1, the urethral-insertion tool 4 is fixed to the frame 2 such that the inclination angle θ2 is fixed, the puncture apparatus 1 is not limited to this, but the inclination angle θ2 may be variable. By this, the inclination angle θ2 can be adjusted in accordance with the patient, and therefore, the puncture apparatus 1 is further improved in convenience in use.

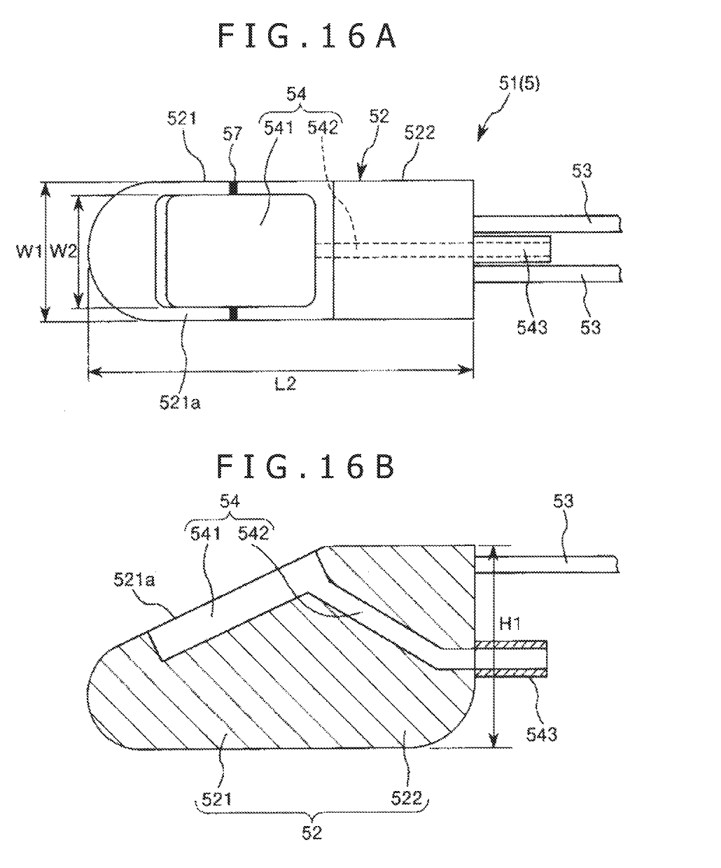

As depicted in FIGS. 12, 16A and 16B, the vaginal-insertion tool 5 has an elongated vaginal-insertion portion 51 for being inserted up to the middle thereof into the vagina, and a supporting portion (vaginal-insertion portion supporting portion) 50 for supporting the vaginal-insertion portion 51. Further, the vaginal-insertion portion 51 has a distal portion 52 positioned on the distal side and a shaft portion 53 connected to a proximal portion of the distal portion 52, the shaft portion 53 being supported by the supporting portion 50.

As the constituent materials of the vaginal-insertion portion 51 and the supporting portion 50, there is no particular restriction, and various metal materials such as, for example, stainless steel, aluminum or aluminum alloys, or titanium or titanium alloys or various resin materials can be used, like in the case of the urethral-insertion tool 4 (the urethral-insertion portion 41 and the supporting portion 40).

The distal portion 52 is a portion for being inserted into the vagina. Although the length L2 of the distal portion 52 is not restricted particularly, it preferably is, for example, approximately 20 mm to 100 mm, and more preferably is approximately 30 mm to 60 mm. Further, although the width W1 of the distal portion 52 is not restricted particularly, it preferably is, for example, approximately 10 mm to 50 mm, and more preferably is approximately 20 mm to 40 mm. By adopting such a length (L2) and a width (W1) as given above, the distal portion 52 is provided with a shape and a size suitable for most vaginas. Therefore, the stability of the puncture apparatus 1 in the mounted state can be enhanced and the burden on the patient can be reduced.

Further, the distal portion 52 has a substantially fixed width as a whole and has a rounded distal portion. Further, the distal portion 52 has a tapering portion 521 positioned on the distal side and having a height gradually increasing toward the proximal side, and an inflow reduction portion 522 positioned on the proximal side of the tapering portion 521 and having a substantially fixed height. Note that the inflow reduction portion 522 has a function of suppressing inflow of air into the vagina through the vaginal orifice in a state in which the distal portion 52 is inserted in the vagina.

In the tapering portion 521, an upper face (face on the urethral-insertion portion 41 side) 521a is inclined with respect to the urethral-insertion portion 41 such that it is spaced away from the urethral-insertion portion 41 toward the distal end. Consequently, in comparison with an alternative case in which the upper face 521a is not inclined, the positional relationship between the urethral-insertion portion 41 and the distal portion 52 can be made closer to the actual positional relationship between the urethra and the vagina. Therefore, in the mounted state, the puncture apparatus 1 can be stably held on the patient and the burden on the patient can be reduced.

Although an inclination angle θ3 of the upper face 521a with respect to the urethral-insertion portion 41 is not restricted particularly, it preferably is, for example, approximately 0 degrees to 45 degrees, and more preferably is approximately 0 degrees to 30 degrees. This setting enables the aforesaid effects to be exhibited more remarkably. In contrast, if the inclination angle θ3 is smaller than the aforesaid lower limit value or is greater than the aforesaid upper limit value, then depending upon the individual differences of the patient, the posture during the procedure or the like, the vagina or the urethra is deformed unnaturally in the mounted state, and the puncture apparatus 1 may not be stably held.

Meanwhile, in the distal portion 52, a suction hole 54 is provided as a suction portion for absorbing the vaginal wall to the distal portion 52.

The suction hole 54 has a bottomed recess 541 which is open to the upper face 521a of the tapering portion 521. Consequently, as hereinafter described, the vaginal anterior wall can be absorbed to the distal portion 52. Further, the suction hole 54 is connected to a suction port 543 provided at a proximal portion of the distal portion 52 through the inside of the distal portion 52.

The suction port 543 is provided so as to be positioned outside a living body in the mounted state. To the suction port 543, a suction device such as a pump can be connected. If the suction device is rendered operative in a state in which the distal portion 52 is inserted in the vagina, then the vaginal anterior wall, which is an upper face of the vaginal wall is absorbed and fixed to the recess 541. If the vaginal-insertion portion 51 is pushed in to the inner side of the body (distal side of the vaginal-insertion portion 51) in a state in which the vaginal anterior wall is absorbed and fixed to the recess 541, then the vaginal wall can be pushed in together with this. Therefore, the disposition or the shape of the vaginal wall can be arranged, and the puncture route for the puncture member 3 can be sufficiently secured. As a result, puncture by the puncture member 3 can be performed relatively accurately and safely.

Note that the number of suction holes 54 is not limited to one but a plurality of suction holes 54 may be provided. Where a plurality of suction holes 54 are provided, the disposition of the recess 541 is not limited particularly but the recesses 541 may be disposed in a juxtaposed relationship in the lengthwise direction or may be disposed in a juxtaposed relationship in a widthwise direction. Alternatively, the recesses 541 may be disposed in a juxtaposed relationship in both of the lengthwise direction and the widthwise direction and hence in a matrix or may be disposed irregularly.

As depicted in FIG. 12, a region S2 in which the recess 541 is formed is opposed to the region S1 in which the suction holes 44 are formed. Further, the puncture apparatus 1 can be configured such that the needle tip of the puncture member 3 passes between the regions S1 and S2. As described hereinabove, since, in the region S1, the urethral wall is absorbed to the urethral-insertion portion 41 and, in the region S2, the vaginal wall is absorbed to the distal portion 52, between the regions S1 and S2, the urethral wall and the vaginal wall are spaced away from each other by a greater distance and with a higher degree of certainty. By passing the puncture member 3 through such a region as just described, the puncture member 3 can be operated to puncture with a higher degree of safety.

Note that the region S2 preferably extends substantially over an overall area in a widthwise direction of the upper face 521a as depicted in FIG. 16A. Although the width W2 of the region S2 is not restricted particularly, it preferably is, for example, approximately 9 mm to 49 mm, and more preferably is approximately 19 mm to 39 mm. By this, the vaginal wall can be absorbed to the distal portion 52 with a higher degree of certainty without being influenced much by the shape of the vaginal wall.

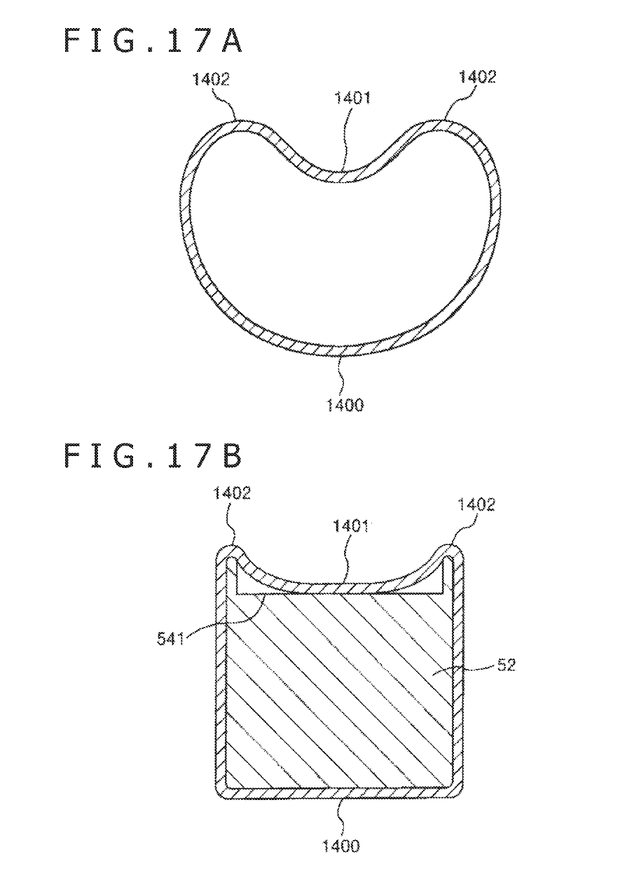

In accordance with an exemplary embodiment, for example, depending upon the patient, a central portion 1401 of the vaginal anterior wall depends downwardly into the vagina such that a vagina 1400 has depressed portions (regions also called “buttonholes”) 1402 on the opposite sides of the central portion 1401. Also in such a case as just described, not only the central portion 1401 but also the depressed portions 1402 can be absorbed with a higher degree of certainty as depicted in FIG. 17B. By absorbing the depressed portions 1402 to the distal portion 52 with a higher degree of certainty, the depressed portions 1402 can be spaced away from the urethra 1300, and the depressed portions 1402 can effectively be prevented from being punctured by the puncture member 3.

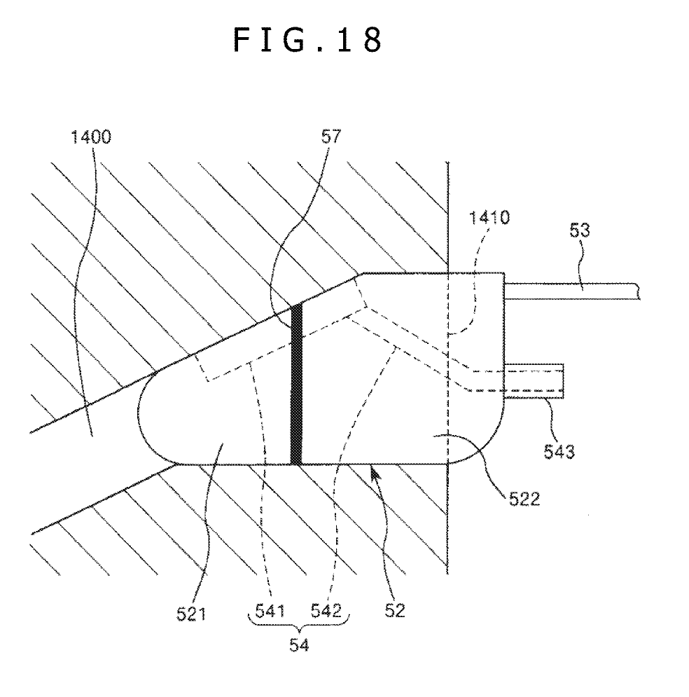

Here, in the state in which the distal portion 52 is inserted in the vagina 1400 as depicted in FIG. 18, the inflow reduction portion 522 is positioned in the proximity of a vaginal orifice 1410 of the vagina 1400 (on the proximal side with respect to the region of the vagina 1400 in which the recess 541 is positioned) and at the vaginal orifice 1410. The inflow reduction portion 522 is higher than the tapering portion 521 on the distal side therefrom and is longer at an overall circumference thereof (transverse area) than the tapering portion 521. Therefore, a portion of the vagina 1400 in the proximity of the vaginal orifice 1410 and the vaginal orifice 1410 extends by a greater amount and are closely contacted with the outer periphery of the inflow reduction portion 522 strongly by the contracting force thereof. Consequently, inflow of air into the vagina 1400 (recess 541) through the vaginal orifice 1410 is suppressed, and absorption of the vaginal wall by the suction hole 54 can be performed with a higher degree of certainty and effectiveness.

Note that the shape of the inflow reduction portion 522 is not restricted to the shape in the present embodiment only if it can demonstrate the aforementioned effect, and for example, the inflow reduction portion 522 may be configured such that the height H1 thereof gradually increases toward the proximal side. In this case, the upper face of the inflow reduction portion 522 may be a face continuing to and having an inclination same as that of the upper face 521a of the tapering portion 521 or may be a discontinuous face having a different inclination.

Further, at the distal portion 52, a marker 57 can be provided such that the puncture route of the puncture apparatus 1 can be confirmed. Since the marker 57 is provided such that the puncture member 3 punctures a region between the vaginal wall positioned thereabove and the urethral wall, by confirming the position of the marker 57, the puncture route of the puncture member 3 can be confirmed. Consequently, the operability and the safety of the puncture apparatus 1 can be improved. In accordance with an exemplary embodiment, the marker 57 is preferably provided on at least one of the bottom face and a side face of the distal portion 52. Since the bottom face or the side face is a face, which can be visually confirmed by the operator even in a state in which the distal portion 52 is inserted in the vagina, by providing the marker 57 at such a position as just described, the puncture route of the puncture member 3 can be confirmed. Further, the insertion depth of the distal portion 52 in the vagina can also be confirmed with the marker 57. In accordance with an exemplary embodiment, for example, it is only necessary for such a marker 57 as just described to be visually observed from the outside, and it can be configured, for example, from a colored portion, a recess or projection or the like.

Although the separated distance D between such a distal portion 52 and a urethral-insertion portion 41 as described above is not restricted particularly, it preferably is, for example, approximately 5 mm to 40 mm with reference to the separated distance between the urethral orifice and the vaginal orifice of most women.

The shaft portion 53 has a form of a thin bar extending substantially in parallel to the urethral-insertion portion 41. Although the length of the shaft portion 53 (separated distance between the distal portion 52 and the supporting portion 50) is not restricted particularly, it preferably is substantially equal to or smaller than, for example, approximately 100 mm and more preferably is approximately 20 mm to 50 mm. By this, the shaft portion 53 can be made with an appropriate length, and the operability of the puncture apparatus 1 can be improved. If the length of the shaft portion 53 exceeds the aforesaid upper limit value, then depending upon the configuration and so forth of the frame 2, the center of gravity of the puncture apparatus 1 is displaced by a great distance from the patient, and the stability of the puncture apparatus 1 in the mounted state may be degraded.

The urethral-insertion tool 4 and the vaginal-insertion tool 5 have been described. The insertion tool 6 having such a urethral-insertion tool 4 and a vaginal-insertion tool 5 as described above further has a maintaining portion 61 for maintaining a relative positional relationship between the urethral-insertion portion 41 and the vaginal-insertion portion 51, and a displacement portion 62 which can move the distal portion 52 of the vaginal-insertion portion 51 closer to the urethral-insertion portion 41 than the relative positional relationship maintained by the maintaining portion 61.

As depicted in FIG. 12, the displacement portion 62 has a hinge portion 621, and the supporting portions 40 and 50 are interlocked with each other for rotary movement by the hinge portion 621. By interlocking the supporting portion 40 with the supporting portion 50 for rotary movement in this manner, the displacement portion 62 of a simple configuration can be obtained, and further, an operation for moving the distal portion 52 toward the urethral-insertion portion 41 can be simply performed.

Meanwhile, the maintaining portion 61 can assume a state in which it fixes the urethral-insertion tool 4 and the vaginal-insertion tool 5 to maintain the relative positional relationship therebetween and another state in which it cancels the fixation of the urethral-insertion tool 4 and the vaginal-insertion tool 5 and allows the vaginal-insertion tool 5 to be moved rotationally with respect to the urethral-insertion tool 4.

As depicted in FIG. 12, the maintaining portion 61 has an operation portion 611 provided on the supporting portion 50, and an engaging portion 612 provided on the supporting portion 40 for engaging with the operation portion 611. Further, the operation portion 611 has a shaft portion 611a which can be flexion-deformed (elastically deformed), a knob 611b interlocked with the shaft portion 611a, and a pawl portion 611c provided at a distal portion of the knob 611b. The engaging portion 612 is configured from a recess 612a formed on the supporting portion 40.

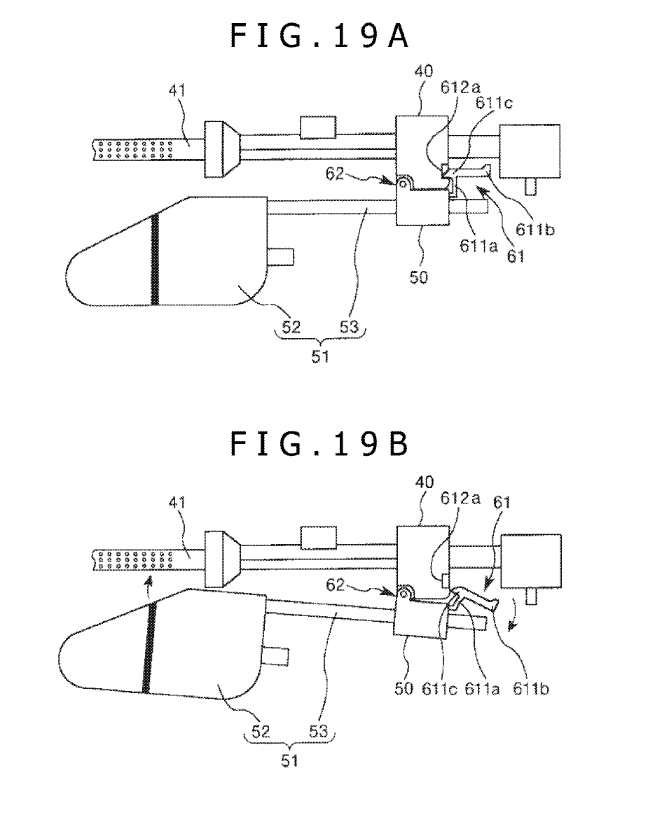

As depicted in FIG. 19A, by placing the pawl portion 611c into a state in which it is engaged with the recess 612a, the supporting portion 50 is fixed to the supporting portion 40, by which the relative positional relationship between the urethral-insertion tool 4 and the vaginal-insertion tool 5 can be maintained. On the other hand, by pulling the knob 611b downwardly to remove the pawl portion 611 c from the recess 612a as depicted in FIG. 19B, the maintenance state is canceled and the vaginal-insertion tool 5 is placed into a state in which it can be moved rotationally with respect to the urethral-insertion tool 4. In the state in which the vaginal-insertion tool 5 can be moved rotationally, the vaginal-insertion portion 51 (distal portion 52) can be moved more closely to the urethral-insertion portion 41 than in the maintenance state depicted in FIG. 19A.

Note that, while the engaging portion 612 in the present embodiment is provided on the supporting portion 40 and the operation portion 611 is provided on the supporting portion 50, conversely the engaging portion 612 may be provided on the supporting portion 50 while the operation portion 611 is provided on the supporting portion 40. Further, the configuration of the operation portion 611 is not limited to that of the present embodiment if a similar effect can be demonstrated.

Further, the insertion tool 6 may further include a biasing portion (for example, a spring) for biasing the vaginal-insertion portion 51 to the urethral-insertion portion 41 side. By this, if the maintaining portion 61 is unlocked, then the vaginal-insertion portion 51 is moved automatically toward the urethral-insertion portion 41 side by the biasing force of the biasing portion. Therefore, an operation of the insertion tool 6 can be further simplified.

As depicted in FIG. 2, the skin cutting knife 11 has a blade portion 111 capable of cutting (dissecting) the surface of a living body, and a grasping portion 112 provided on the proximal side of the blade portion 111.

The blade portion 111 has a form of a plate and has a width decreasing toward the distal side. Further, the blade portion 111 has a sharp cutting edge 1111 at a distal portion thereof. The cutting edge 1111 is formed over the overall length of the blade portion 111 in the widthwise direction. In accordance with an exemplary embodiment, in the skin cutting knife 11, an entire portion at which the width of the blade portion 111 decreases serves as the cutting edge 1111.

The material for configuring the blade portion 111 is not restricted particularly, and for example, various metal materials such as stainless steel can be used.

The grasping portion 112 is a portion for being grasped by an operator. The grasping portion 112 has an elongated shape and has a transverse section of a flattened shape. The grasping portion 112 is greater in both of the width and the thickness than the blade portion 111.

By cutting a portion of the body surface H, at which puncture by the puncture member 3 is to be started, by such a skin cutting knife 11 as described above, a cut skin portion (dissection portion) 2000 is formed. Note that, in the present embodiment, the cut skin portion 2000 is formed at a portion of the body surface H at which puncture by the puncture member 3 is to be completed, namely, also at a portion of the body surface H at which the puncture member 3 inserted in the living body is exposed to the outside of the living body again.

Note that, in the present embodiment, the thickness of the cut skin portion 2000 preferably is, although this depends upon the genetic makeup (thickness of the skin) of the patient, for example, approximately 0.5 mm to 10 mm, and more preferably is approximately 1 mm to 5 mm, which helps make it possible to reduce the invasion by the skin cutting.

The guide member 8 has a function of guiding the cutting edge 1111 of the skin cutting knife 11 when the body surface H is cut by the skin cutting knife 11 before an insertion hole into which the implant 9 is to be inserted is formed in the living body by the puncture apparatus 1.

As depicted in FIG. 2, the guide member 8 is configured from a plate member having a substantially rectangular shape as viewed in plan and is configured as a separate member from the puncture apparatus 1. The length L of the long side of the guide member 8 is substantially equal to the separated distance between the distal end and the proximal end of the puncture member 3.

A marker 83 for indicating a positional relationship with a living body is provided at a central portion of the guide member 8. The marker 83 is configured from a colored portion. In the disposition state, the operator can recognize the center of the living body with reference to the marker 83. The marker 83 is provided in parallel to the short sides of the guide member 8. The marker 83 is provided, in the present embodiment, on the upper face, the lower face and the side faces of the longer side of the guide member 8, which helps enable the operator to visually recognize the marker 83 from any direction in the disposition state.

The marker 83 may be formed, for example, by application of paint (film coating), printing, staining, pasting of a sticking article or fusion. Further, the color of the marker 83 is not restricted particularly, and, for example, may be selected from various chromatic colors, achromatic colors, metal colors, and fluorescent colors. However, the color of the marker 83 preferably is different from colors of peripheral portions of the marker 83 of the guide member 8.

Further, a pair of recesses (guide portions) 81 are formed at the opposite end portions of the guide member 8 such that they are open to side faces. Since the recesses 81 have a similar configuration, in the following description, one of the recesses 81 is described as a representative.

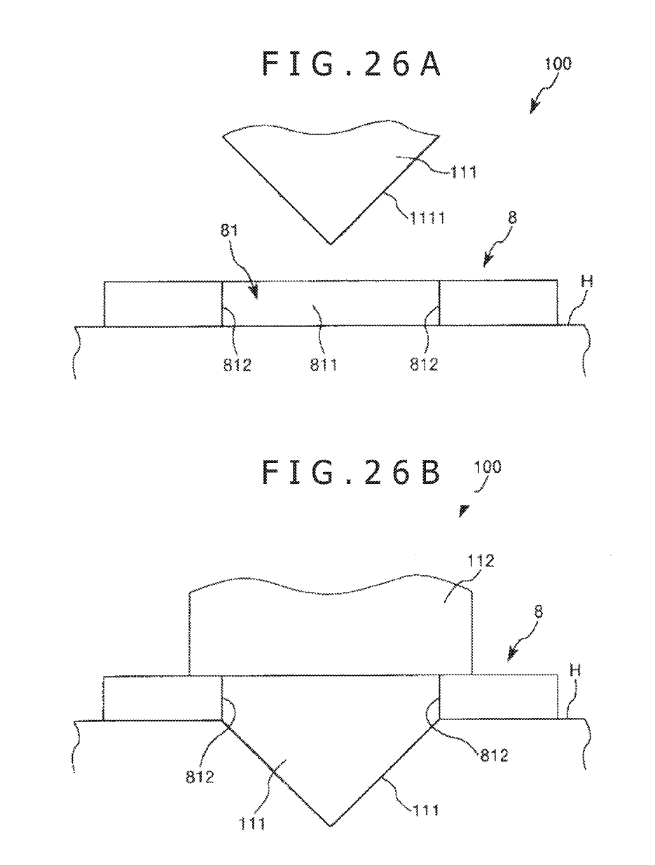

The recess 81 is formed by cutting away a portion of the guide member 8 into a channel shape toward the center side and formed as a through-hole extending through the guide member 8 also in the thicknesswise direction. As depicted in FIG. 26A, the recess 81 has a first inner side face 811 positioned on the center side of the guide member 8 and a pair of second inner side faces 812 continuous to the opposite ends of the first inner side face 811 and opposing to each other.

The material for configuring such a guide member as described above is not restricted particularly, and for example, various resin materials, various metal materials and so forth can be used as the material.

Now, usage of the puncture apparatus 1, namely, a method (procedure) of embedding the implant 9 into a living body using the puncture apparatus 1, is described.

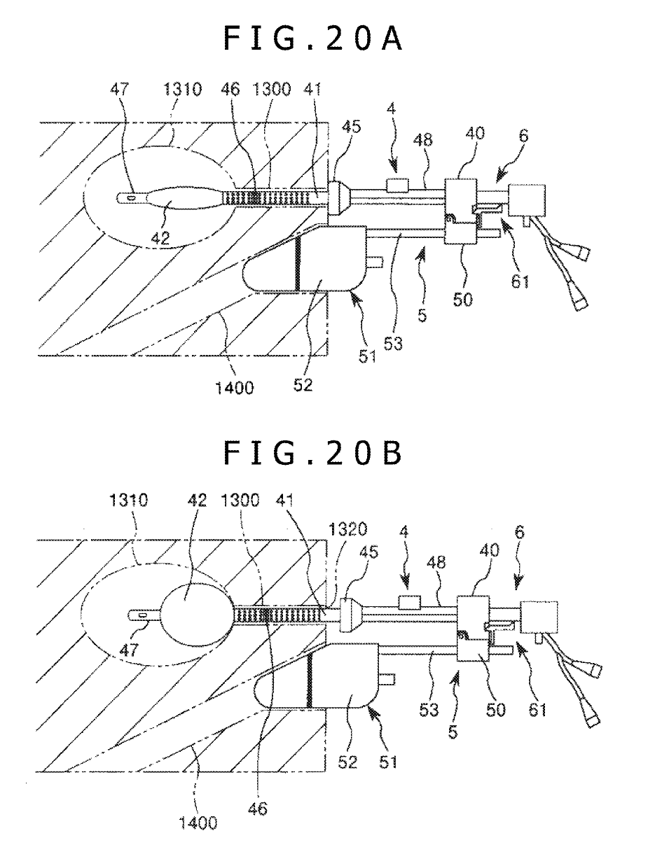

First, the patient is set to a lithotomy position on an operating table, and the insertion tool 6 is mounted on the patient as depicted in FIG. 20A.

In particular, the urethral-insertion portion 41 of the urethral-insertion tool 4 is first inserted into the urethra 1300 of the patient and the vaginal-insertion portion 51 of the vaginal-insertion tool 5 is inserted into the vagina 1400 of the patient. By inserting the urethral-insertion portion 41 into the urethra 1300, the urethra 1300 is corrected to its predetermined shape (in the present embodiment, into a linear shape) by the urethral-insertion portion 41, and by inserting the vaginal-insertion portion 51 into the vagina 1400, the vagina 1400 is corrected to its predetermined shape by the vaginal-insertion portion 51 (distal portion 52).

Then, the balloon 42 disposed in the bladder 1310 is expanded and the urine is drained from within the bladder 1310 through the urine drain hole 471 as occasion demands. Then, as depicted in FIG. 20B, the urethral-insertion portion 41 is pulled to the outer side of the body (to the proximal side) until the balloon 42 attaches the bladder neck, and in this state, it is confirmed whether or not the marker 46 is exposed from the urethral orifice 1320. As described hereinabove, where the marker 46 is exposed from the urethral orifice 1320, it can be decided that the puncture apparatus 1 should not be used, and at this point of time, the procedure is discontinued. In contrast, where the marker 46 is not exposed from the urethral orifice 1320, it can be decided that the puncture apparatus 1 can be used, and the procedure is continued.

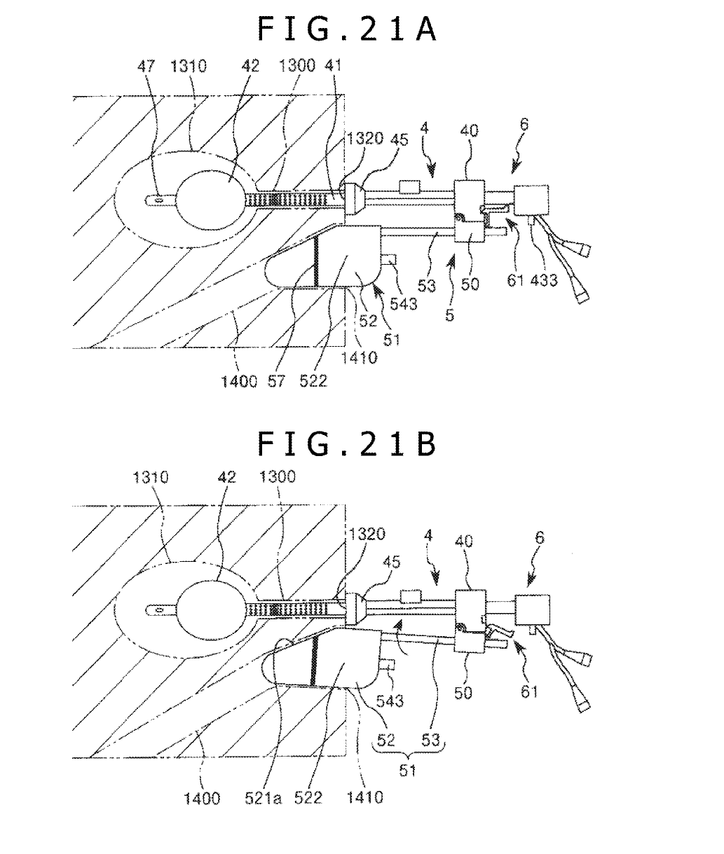

Then, the urethral-insertion portion 41 is pushed in to the inner side of the body until the marker 45 attaches the urethral orifice 1320 as depicted in FIG. 21A. Consequently, the puncture member 3 is positioned with respect to the urethral orifice 1320, and the puncture route of the puncture member 3 is defined. Here, by further confirming the puncture route of the puncture member 3 using the marker 57 provided at the distal portion 52 of the vaginal-insertion portion 51, a relatively safer procedure can be performed.

Then, suction devices are connected to the suction ports 433 and 543 and are rendered operative to suck the urethral wall to the suction holes 44 of the urethral-insertion portion 41 and absorb the vaginal wall to the suction hole 54 of the vaginal-insertion portion 51. As described hereinabove, since, at this time, on the urethra 1300 side, the marker 45 covers and closes up the urethral orifice 1320, inflow of air into the urethra 1300 through the urethral orifice 1320 is suppressed, and the absorption of the urethral wall to the urethral-insertion portion 41 can be performed with a higher degree of certainty. Meanwhile, on the vagina 1400 side, since the vaginal orifice 1410 closely contacts with the inflow reduction portion 522, inflow of air into the vagina 1400 through the vaginal orifice 1410 is suppressed, and the absorption of the vaginal wall to the vaginal-insertion portion 51 can be performed with a higher degree of certainty.

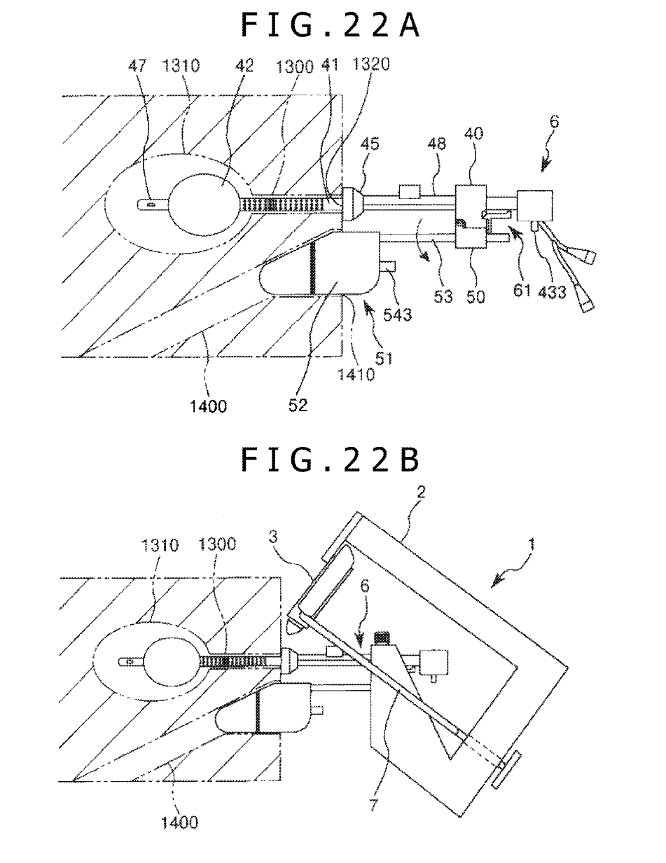

In accordance with an exemplary embodiment, in the vaginal-insertion tool 5, the maintaining portion 61 is unlocked first as depicted in FIG. 21B to place the vaginal-insertion portion 51 into a state in which it can move rotationally with respect to the urethral-insertion portion 41. Then, the vaginal-insertion portion 51 is moved to the urethral-insertion portion 41 side. Consequently, the upper face 521a of the distal portion 52 and the vaginal anterior wall can be closely contacted with a higher degree of certainty with each other. In this state, by rendering the suction device connected to the suction port 543 operative, the vaginal wall can be absorbed to the vaginal-insertion portion 51 with a higher degree of certainty. After the vaginal wall is absorbed, the vaginal-insertion portion 51 is moved in a direction away from the urethral-insertion portion 41 as depicted in FIG. 22A, and the maintaining portion 61 is locked to fix the relative positional relationship of the vaginal-insertion portion 51 and the urethral-insertion portion 41. Consequently, the state in which the urethra 1300 and the vagina 1400 are sufficiently spaced away from each other can be maintained.

Upon such absorption as described above, for example, if the urethral wall is absorbed precisely to the urethral-insertion portion 41, then since the suction holes 44 is closed up with the urethral wall, the suction from the suction port 433 is stopped or weakened. Similarly, if the vaginal wall is precisely absorbed to the vaginal-insertion portion 51, then since the suction hole 54 is closed up with the vaginal wall, the suction from the suction port 543 is stopped or weakened. Therefore, the operator can confirm whether or not the urethral wall and the vaginal wall are precisely absorbed to the urethral-insertion portion 41 and the vaginal-insertion portion 51 from suction conditions (for example, magnitudes of sound generated by the suction) from the suction ports 433 and 543, respectively.

Note that the insertion tool 6 may have a confirmation portion for mechanically confirming an absorption state. Although the confirmation portion is not restricted, particularly, if it only allows confirmation of the absorption state, for example, it can be configured such that it has a flow range measurement unit (negative pressure gauge) for measuring the flow range from the suction port 543 and a decision unit for deciding whether or not absorption is performed precisely on the basis of a measurement result from the flow range measurement unit.

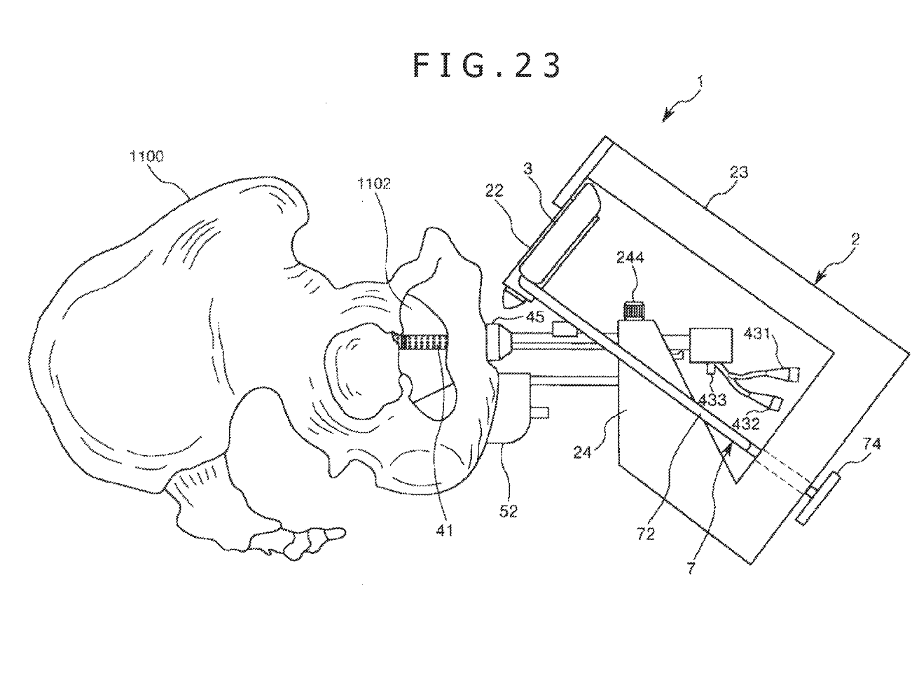

After the urethral posterior wall and the vaginal anterior wall are placed into the state in which they are spaced away from each other sufficiently to allow the puncture member 3 to puncture in such a manner as described above, the frame 2 is fixed to the insertion tool 6 as depicted in FIG. 22B. Consequently, a state in which the puncture apparatus 1 is mounted on the patient can be established. In this state, the positional relationship between the pelvis 1100 and the puncture apparatus 1 is such a state as depicted in FIG. 23.

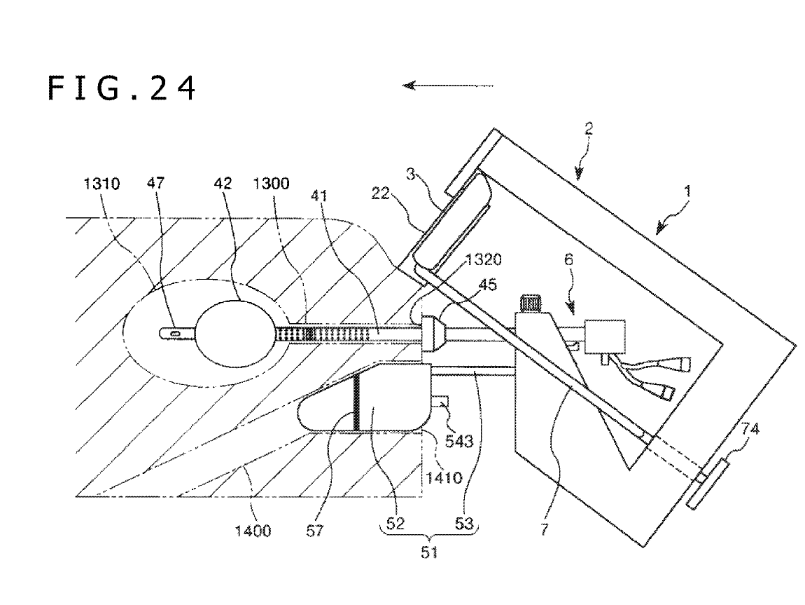

Then, the restriction portion 48 is removed from the urethral-insertion portion 41 as depicted in FIG. 24, and the insertion tool 6 is pushed in to the inner side of the body. Since the urethral wall is absorbed to the urethral-insertion portion 41 and the vaginal wall is absorbed to the vaginal-insertion portion 51 as described hereinabove, when the insertion tool 6 is pushed in to the inner side of the body, the urethra 1300 and the vagina 1400 are pushed in together. Therefore, puncture of the tissue by the puncture member 3 can be further facilitated.

In accordance with an exemplary embodiment, for example, in the present embodiment, since the sliding resistance amplification portion 49 for increasing the sliding resistance with respect to the urethral is provided on the urethral-insertion portion 41, the urethra 1300 can be pushed in with a higher degree of certainty. Further, since rubbing (displacement) of the urethral-insertion portion 41 and the urethra 1300 against (from) each other is reduced, damage to the urethra can be reduced.

Note that, at this time, since the restriction portion 48 is removed, the marker 45 is slidably moved toward the proximal side of the urethral-insertion portion 41 while attaching the urethral orifice 1320, and this does not obstruct the pushing in of the urethral-insertion portion 41.

Further, while a case in which both of the urethra 1300 and the vagina 1400 are pushed in to the inner side of the body is described in the foregoing description, only one of the urethra 1300 and the vagina 1400 may be pushed in to the inner side of the body. Also by this, a similar effect can be demonstrated.

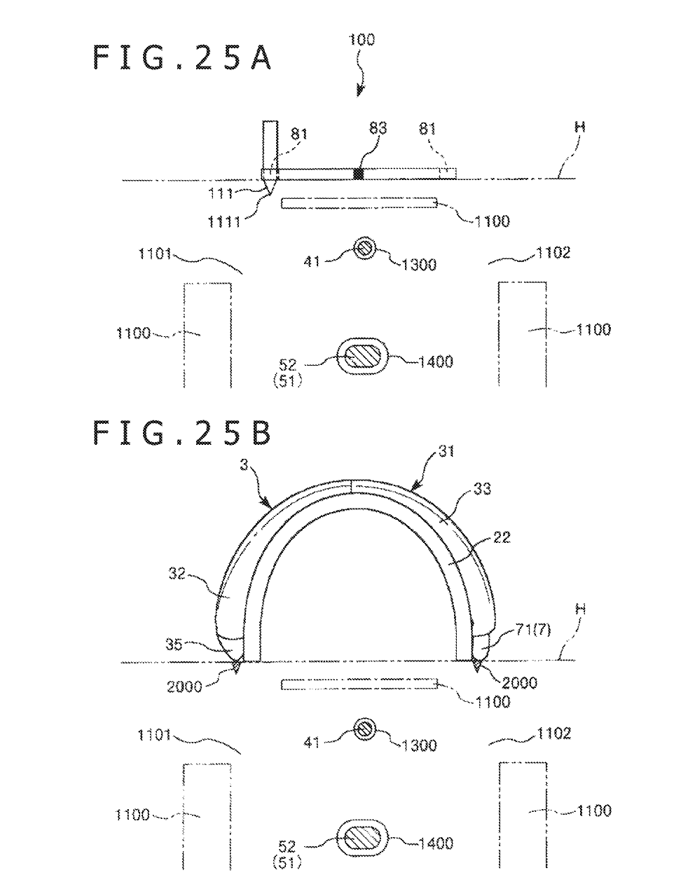

Then, the guide member 8 is disposed on the body surface H to establish the disposition state as depicted in FIG. 25A. At this time, positioning of the guide member 8 with respect to the center of the living body can be performed with reference to the marker 83 of the guide member 8.

Then, the skin cutting knife 11 is inserted into one of the recesses 81 of the guide member 8 as depicted in FIG. 26A. At this time, since the blade portion 111 of the skin cutting knife 11 is guided by the recess 81 (first inner side face 811), the skin cutting operation can be performed relatively easily and accurately to form a cut skin portion 2000. Further, in the state in which the blade portion 111 is inserted in the recess 81, a distal surface of the grasping portion 112 of the skin cutting knife 11 is attached to an edge portion of the recess 81 on the upper face of the guide member 8 (refer to FIG. 26B). Consequently, the insertion depth of the cutting edge 1111 into the living body is restricted, and the cut skin portion 2000 can be prevented from becoming excessively great (deep). Therefore, a skin cutting operation can be performed with low invasion and relatively safe.

Similarly, the blade portion 111 is inserted also into the other recess 81 to form a cut skin portion 2000.

Note that, in order to form a cut skin portion 2000 by a skin cutting knife having a blade portion of a width smaller than that of the blade portion 111 depicted in FIGS. 26A and 26B, after the blade portion is inserted, it is moved in the recess 81 to form a cut skin portion 2000. In this case, since the second inner side faces 812 of the recess 81 is attached to the cutting edge 1111, the length of the cut skin portion 2000 can be prevented from becoming excessively great.

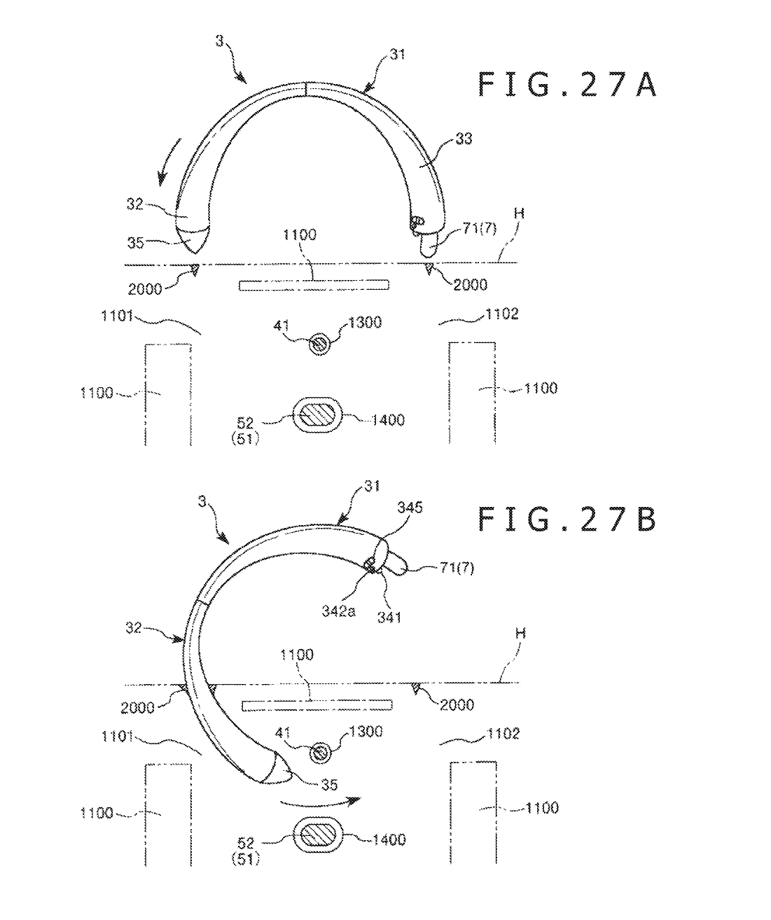

Then, the guide member 8 is removed from the body surface H and the opposite end portions of the guide portion 22 are attached to the body surface H (refer to FIG. 25B). Then, the handle 74 is operated to turn the operation member 7 as depicted in FIGS. 27A and 27B so that the puncture route of the puncture member 3 may pass the safety zones S5 of the left and right obturator foramens 1101 and 1102 of the pelvis 1100.

Since the length L of the long side of the guide member 8 is substantially equal to the separated distance between the distal end and the proximal end of the puncture member 3 as described hereinabove, the cut skin portions 2000 are positioned substantially just below the needle tip 351. Consequently, the needle tip 351 is inserted smoothly into the cut skin portion 2000 on the left side in FIG. 27B. As a result, the needle tip 351 can pass the body surface H from the cut skin portion 2000 with a comparatively small force.

The needle body 35 of the puncture member 3 punctures the body surface H at the right side inguinal region of the patient or in a region in the proximity of the right side inguinal region and enters the body. Then, the needle body 35 successively passes the obturator foramen 1101, a region between the urethra 1300 and the vagina 1400 and the other obturator foramen 1102, and then projects to the outside of the body from the body surface H at the left side inguinal region or in a region in the proximity of the left side inguinal region. At this time, since the needle tip 351 passes the cut skin portion 2000 on the right side in FIG. 27B, it can pass the body surface H with a comparatively small force.

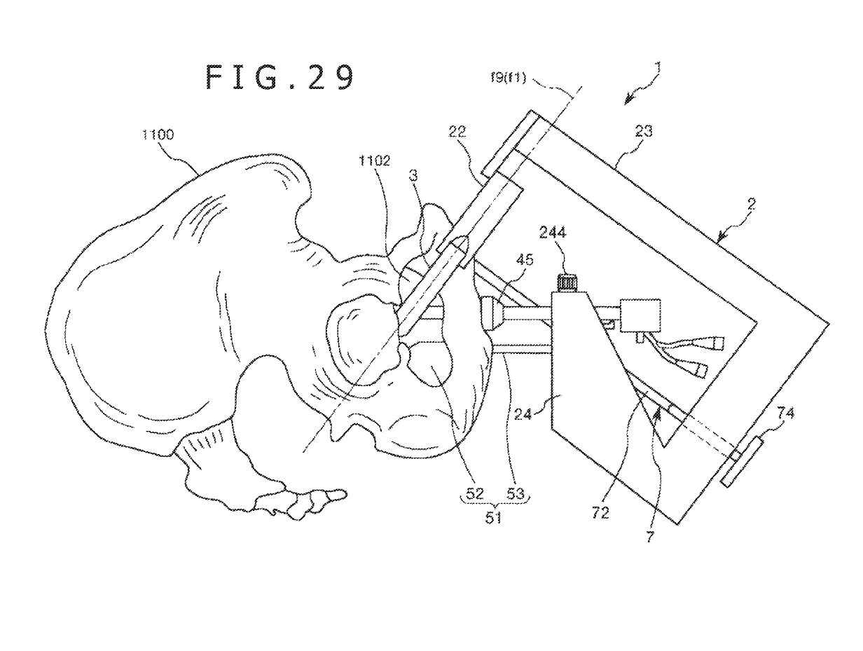

If the puncture member 3 punctures substantially vertically with respect to the left and right obturator foramens 1101 and 1102 of the pelvis 1100, then the route can be formed at a position suitable for indwelling of the implant 9. In this state, the positional relationship between the pelvis 1100 and the puncture apparatus 1 is such a state as depicted in FIG. 29.

Further, thereupon, the puncture member 3 punctures the living body such that it passes between the marker 46 and the urethral orifice 1320. Consequently, the puncture member 3 punctures a region between a middle portion of the urethra 1300 (middle urethra) and the vagina 1400, and a more effective treatment can be performed.

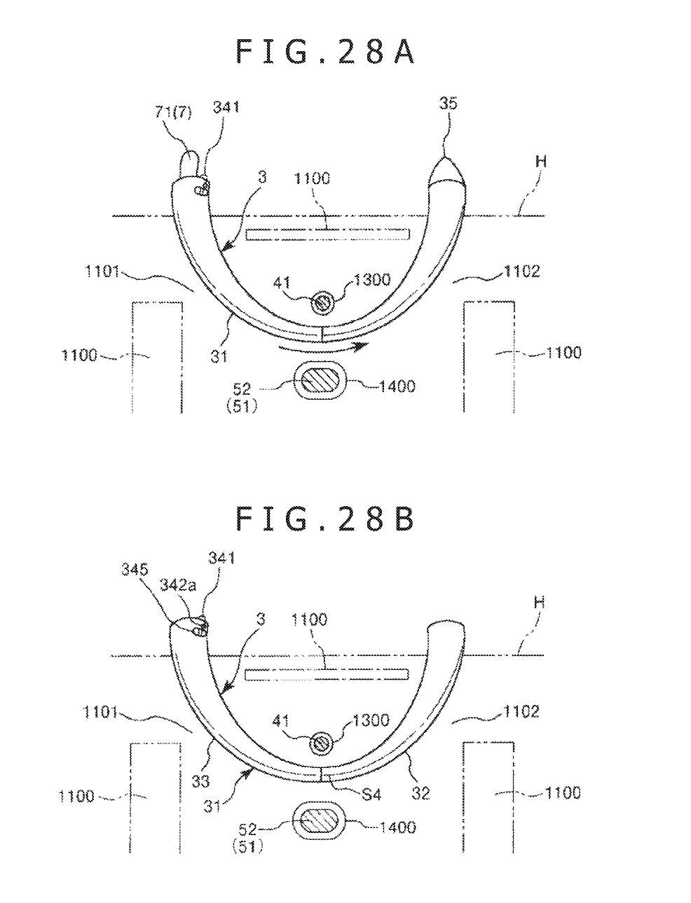

The puncture member 3 is disposed in the living body thereby. Then, the handle 74 is operated to turn the operation member 7 in the reverse direction to pull out only the operation member 7 from within the living body while the puncture member 3 remains disposed in the living body. Thereafter, the frame 2 is removed from the insertion tool 6, and the needle body 35 is removed from the main body 31 further. Consequently, a state in which only the main body 31 is disposed in the living body is established as depicted in FIG. 28B. The main body 31 is disposed in the living body in the state in which both of the distal side opening and the proximal side opening thereof are exposed to the outside of the living body.

Then, the position of the main body 31 can be adjusted as needed. In particular, the left and right projection lengths of the main body 31 can be adjusted to each other to position the central portion S4 of the main body 31 between the urethra 1300 and the vagina 1400. In this state, as depicted in FIG. 28B, the widthwise direction (direction of the major axis J32) W of the central portion S4 is disposed substantially in parallel to the urethra 1300. In other words, the urethra 1300 in and by which the urethral-insertion tool 4 is inserted and corrected and the widthwise direction W of the central portion S4 are positioned substantially in parallel to each other.

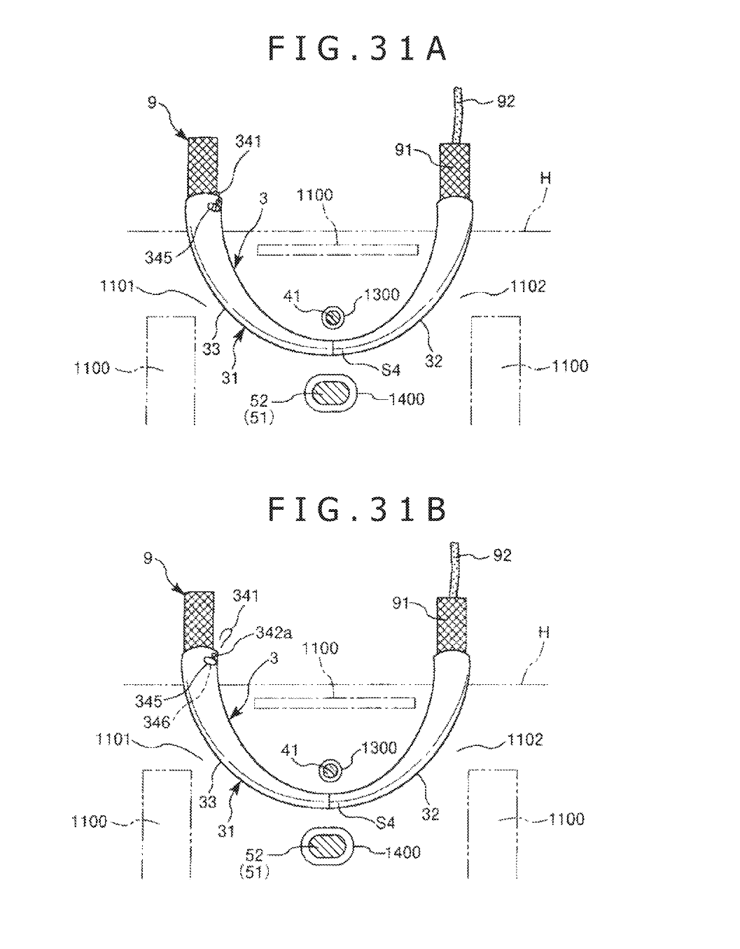

Then, the implant 9 is extracted from the packaging material 90 and inserted into the main body 31 to establish a state in which the implant main body 91 projects from the proximal side opening and the distal side opening of the main body 31 as depicted in FIG. 31A. By keeping the packaging material 90 accommodated until a point of time immediately before it is disposed into the main body 31 in this manner, contamination to the implant 9 can be prevented. Note that, since the main body 31 has a flattened shape as described hereinabove, the posture of the implant main body 91 follows the flattened shape. In accordance with an exemplary embodiment, for example, the implant main body 91 is disposed into the main body 31 such that the widthwise direction thereof coincides with the widthwise direction of the main body 31. From the relationship to the urethra 1300, the implant main body 91 is disposed in parallel to the corrected urethra 1300.

Then, the string 341 exposed from the exposure holes 345 and 346 is cut as depicted in FIG. 31B. Consequently, the main body 31 is placed into a state in which it can be divided into the distal end divisional piece 32 and the proximal end divisional piece 33. Then, the absorption of the urethral wall by the urethral-insertion portion 41 and the absorption of the vaginal wall by the vaginal-insertion portion 51 can be stopped. Consequently, the position and the shape of the urethra 1300 and the vagina 1400 return to their respective original states.

Then, the connection between the distal end divisional piece 32 and the proximal end divisional piece 33 is cancelled, and the distal end divisional piece 32 is pulled out from the living body toward the distal side and the proximal end divisional piece 33 is pulled out from the living body toward the proximal side. Along with this, the distal end divisional piece 32 and the proximal end divisional piece 33 are moved substantially at the same time in the opposite directions to each other so that the distal end divisional piece 32 and the proximal end divisional piece 33 are moved arcuately along the individual shape. Consequently, the main body 31 is removed smoothly from the living body.

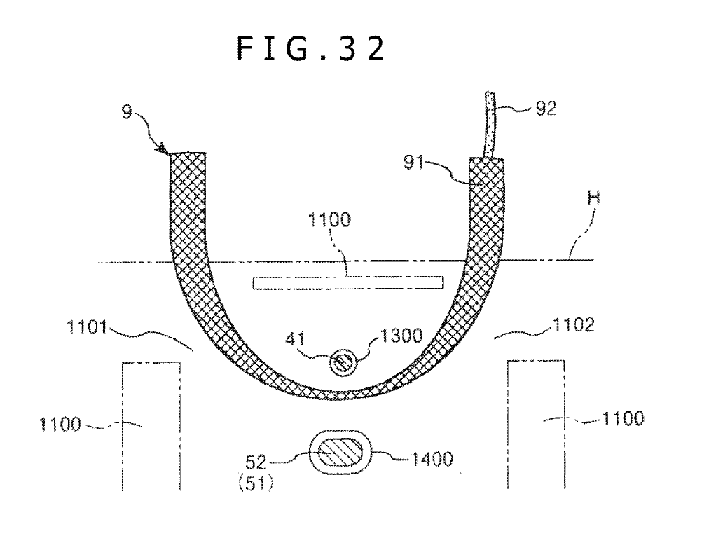

When the distal end divisional piece 32 and the proximal end divisional piece 33 are removed from the living body in such a manner as described above, the surrounding tissue which has been expanded by the main body 31 restores to its original position, and the tissue is gradually contacted with the implant main body 91 from a central portion toward the opposite end portions of the implant main body 91. From the fact that the distal end divisional piece 32 and the proximal end divisional piece 33 are moved in a direction along the shape thereof and the fact that the main body 31 has an internal space in which the implant main body 91 can move with sufficiently low friction as described above, the implant main body 91 can be indwelled in its state without being acted upon by unnecessary tensile force. Consequently, the necessity for adjustment of the tension of the implant main body 91 can be eliminated. From the foregoing, a state in which the implant main body 91 is embedded in the living body as depicted in FIG. 32 is established.

In the state in which the implant 9 is embedded in the living body, the implant main body 91 is disposed substantial in parallel to the urethra 1300 in a region between the urethra 1300 and the vagina 1400. Therefore, the urethra 1300 can be supported over a wider area by the implant main body 91.

By dividing and removing the main body 31 from the living body in this manner, removal of the main body 31 from the living body can be performed relatively easily. Further, since the divisional pieces 32 and 33 during the removal little have an influence on the posture of the implant main body 91 in the region between the urethra 1300 and the vagina 1400, the implant main body 91 can be embedded in a desired posture.

Further, since the divisional pieces 32 and 33 are removed from the living body in the state in which the urethral-insertion portion 41 is inserted in the urethra 1300, excessive tension can be prevented from being applied to the urethra 1300 by the implant main body 91 to be indwelled in the living body.

Then, the insertion tool 6 is removed from the living body. In particular, the urethral-insertion portion 41 is removed from the urethra 1300 and the vaginal-insertion portion 51 is removed from the vagina 1400. After the urethral-insertion tool 4 is removed, the urethra 1300 returns to a shape of a natural state. However, since the implant main body 91 is embedded in the living body tissue, the urethra 1300 in its natural state and the implant main body 91 can be maintained in a parallel state.

Thereafter, an unnecessary portion of the implant main body 91 is cut away, and the procedure is ended.

As described above, with the puncture apparatus 1, when the implant 9 is to be indwelled, this can be coped with only by a procedure of minimal invasion such as puncture by the puncture member 3 and incision or the like which is large in invasion need not be performed. Therefore, the burden on the patient is relatively low and the safety of the patient is relatively high as well. Further, since the implant main body 91 can be embedded in parallel to the urethra 1300, the urethra 1300 can be supported over a wider area. Further, the living body can be punctured by the puncture member 3 while avoiding the urethra 1300 and the vagina 1400. Further, the implant 9 can be prevented from being exposed to the inside of the vagina from a wound caused by incision of the vagina as in a conventional case in which the vagina is incised or complications such as infection from the wound can occur. The puncture apparatus 1 is relatively safe and can embed the implant 9 with relative certainty.

Further, with the guide member 8, a cut skin portion 2000 of an appropriate size can be formed easily and accurately. Further, since it is possible to prevent the operator from forming a cut skin portion 2000 of an unnecessarily great size in error, the procedure can be performed with low invasiveness.

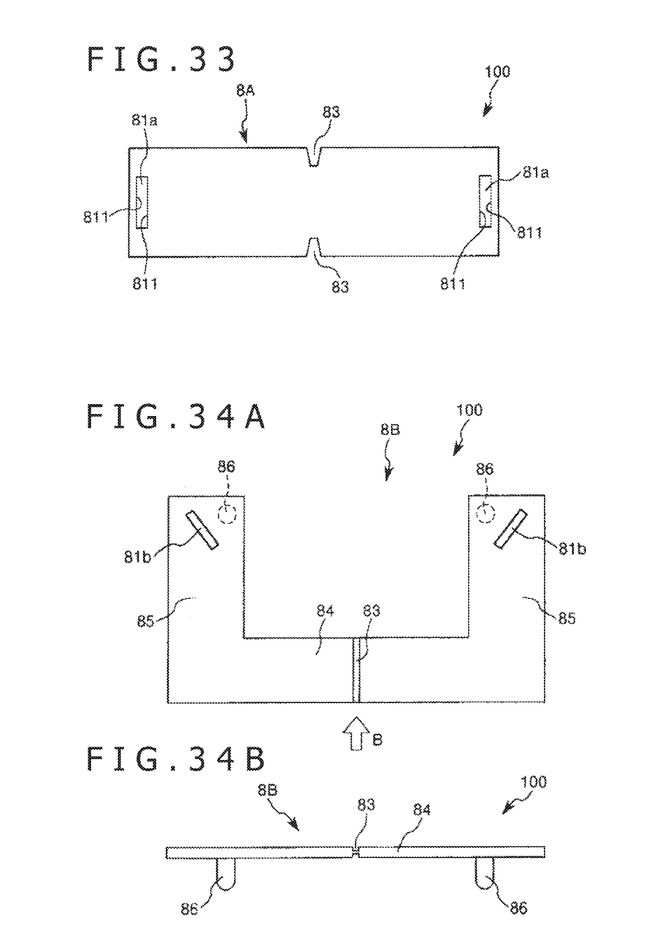

FIG. 33 is a plan view of a guide member provided on a medical tool according to a second embodiment of the present disclosure.

Although the second embodiment of the medical tool of the present disclosure is described below with reference to FIG. 33, the description is given principally of differences of the second embodiment from the aforementioned first embodiment while description of like matters are omitted herein to avoid redundancy.

The present embodiment is similar to the first embodiment except that it is different in configuration (shape) of the guide member.

As depicted in FIG. 33, a guide member 8A has recesses 81a closed at the side faces of the guide member 8A. Consequently, a pair of first inner side faces 811 are formed. Therefore, as the number of first inner side faces 811 is greater by one, the blade portion 111 is guided with a higher degree of certainty into the recess 81a.

Further, a pair of markers 83 are provided at a substantially central portion of the guide member 8A in an opposing relationship in the widthwise direction of the guide member 8A. The markers 83 are formed by cutting away the guide member 8A in a substantially V shape. Therefore, the markers 83 can be easily formed in comparison with those in a case in which the markers 83 are configured from colored portions.

FIGS. 34A and 34B depict a guide member provided on a medical tool according to a third embodiment of the present disclosure, wherein FIG. 34A is a plan view of the guide member and FIG. 34B is a view as viewed in a direction indicated by an arrow mark B in FIG. 34A.

Although the third embodiment of the medical tool of the present disclosure is described below with reference to FIGS. 34A and 34B, the description is given principally of differences of the third embodiment from the aforementioned embodiments while description of like matters are omitted herein to avoid redundancy.

The present embodiment is similar to the second embodiment except that it is different in configuration of the guide member.

As depicted in FIG. 34A, a guide member 8B has an elongated base portion 84, and a pair of projecting pieces 85 projecting from the opposite ends of the base portion 84. In particular, the guide member 8B has a channel shape as viewed in plan.

The projecting pieces 85 project in directions same as each other and substantially perpendicularly to the extending direction of the base portion 84. Further, the projecting pieces 85 have a thickness equal to that of the base portion 84.

A recess 81b is provided in each of the projecting pieces 85. The recess 81b is inclined in accordance with the inclination of the puncture member 3 (sheath 30). Consequently, a cut skin portion 2000 into which the sheath 30 can be inserted can be easily formed. Therefore, the cut skin portion 2000 can be prevented from being unnecessarily expanded by the sheath 30. As a result, an insertion hole can be formed with lower invasion.

Depending upon the constitution of the patient, such a guide member 8B as described above may be used such that, in the disposition state, the body surface H is pressed against the pelvis 1100 side. Since the pubis or the like is positioned under the body surface H positioned between the projecting pieces 85, the portion of the body surface H is a portion which can be pressed down but comparatively difficultly. However, when the guide member 8B is pressed toward the living body, the portion which can be pressed down comparatively difficultly can enter between the projecting pieces 85. That is, the portion between the projecting pieces 85 functions as an escape portion into which a central portion of the pelvis 1100 (for example, the pubis and so forth) and the living body tissue which surrounds the same are admitted. By this, the guide member 8B can be pressed sufficiently toward the living body.

Further, a pair of protrusions 86 are provided at portions of the face, which is attached to the body surface H, of the projecting pieces 85 in the proximity of the recesses 81b as depicted in FIG. 34B. The pair of protrusions 86 press portions, on the pubic symphysis 1200 side, of edge portions of the obturator foramen 1101 and the obturator foramen 1102 of the pelvis 1100 through the living body tissue which covers the pelvis 1100 from the body surface H when the guide member 8B is pressed against the living body in the disposition state. The protrusions 86 thus hold therebetween the portion of the pelvis 1100 between the obturator foramen 1101 and the obturator foramen 1102. In this state, the guide member 8B is restricted against movement in the leftward and rightward direction and the upward and downward direction in FIG. 34A. As a result, the guide member 8B can be easily positioned with respect to the living body.

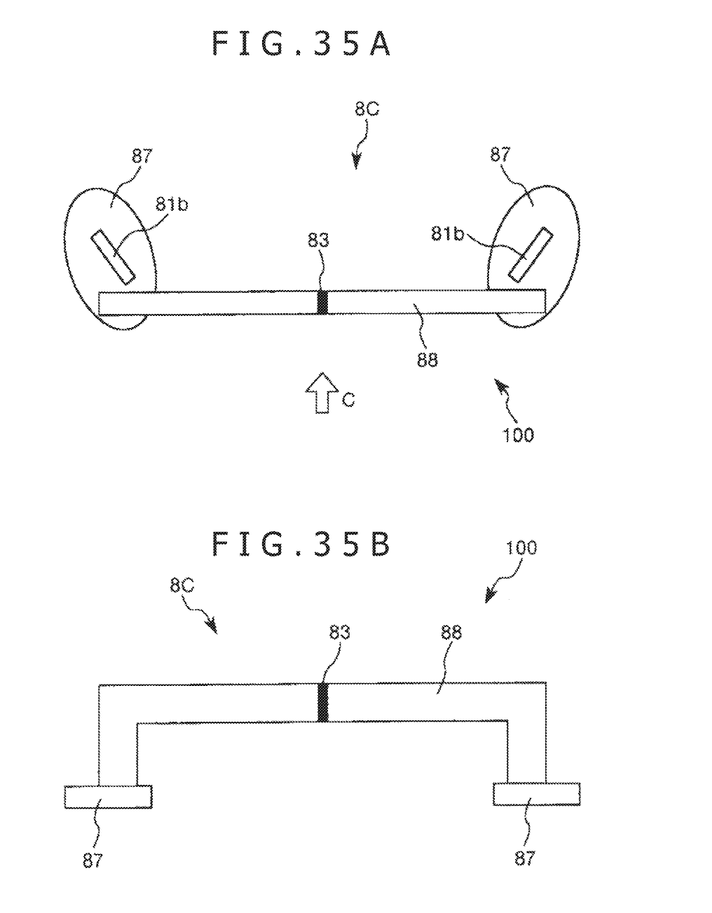

FIGS. 35A and 35B depict a guide member provided on a medical tool according to a fourth embodiment of the present disclosure, wherein FIG. 35A is a plan view of the guide member and FIG. 35B is a view as viewed in a direction indicated by an arrow mark C in FIG. 35A.

Although the fourth embodiment of the medical tool of the present disclosure is described below with reference to FIGS. 35A and 35B, the description is given principally of differences of the fourth embodiment from the aforementioned embodiments while description of like matters are omitted herein to avoid redundancy.

The present embodiment is similar to the third embodiment except that it is different in configuration of the guide member.

As depicted in FIG. 35A, a guide member 8C can include a pair of plate pieces 87, and an interlock portion 88 for interlocking the plate pieces 87 to each other.

Each of the plate pieces 87 has an elliptical shape as viewed in plan and has a recess 81b formed at a central portion thereof. Further, each of the plate pieces 87 is interlocked with the interlock portion 88 such that the recess 81b is inclined in accordance with the inclination of the puncture member 3 (sheath 30).

Further, as depicted in FIG. 35B, the interlock portion 88 has a bar-like shape and is bent at two locations thereof in the longitudinal direction such that it generally has a channel-shaped outer shape. Consequently, the guide member 8C can function as the aforementioned escape portion when the plate pieces 87 are pressed against the living body side.

Further, in the disposition state, the interlock portion 88 can function as a grasping portion since it is spaced away from the body surface H. Consequently, for example, when the guide member 8C is positioned with respect to the living body, the operation can be performed relatively easily.

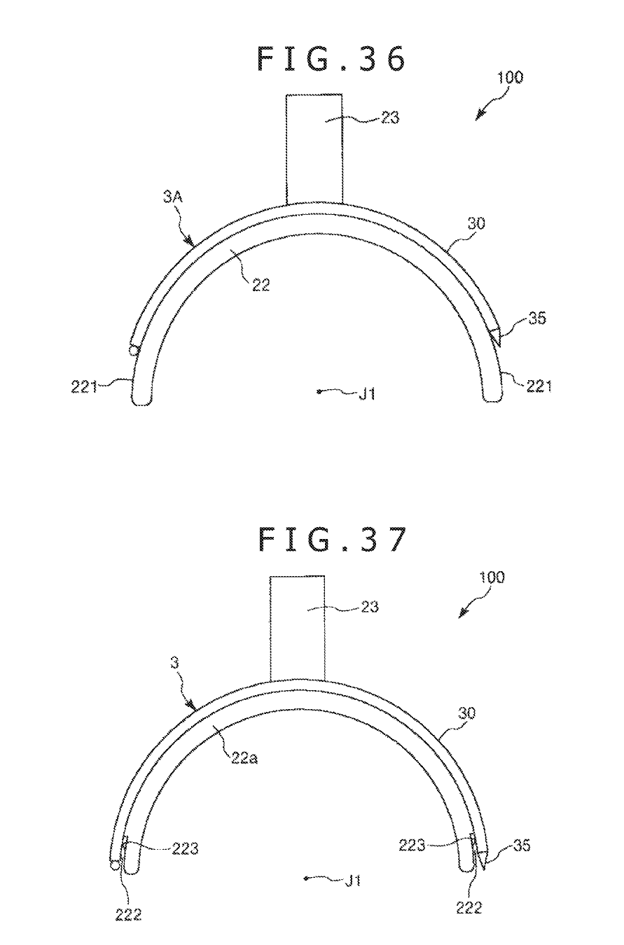

FIG. 36 is a view depicting a guide portion provided on a medical tool according to a fifth embodiment of the present disclosure.

Although the fifth embodiment of the medical tool is described below with reference to FIG. 36, the description is given principally of differences of the fifth embodiment from the aforementioned embodiments while description of like matters are omitted herein to avoid redundancy.

Note that FIGS. 36 to 46B schematically depict a guide portion and a puncture member in order to facilitate understandings. Further, FIGS. 36 to 46B are views as viewed from the handle 74 side of the puncture apparatus 1.

The present embodiment is similar to the first embodiment except that it is different in configuration of the guide portion.

As depicted in FIG. 36, a puncture member 3A (sheath 30) is shorter than the puncture member 3 of the first embodiment. Consequently, the opposite end portions of a guide portion 22 are exposed. In a state in which the opposite end portions of the guide portion 22 are attached to the body surface H, skin cutting can be carried out by moving the blade portion 111 along the face on the outer sides of exposed portions 221, namely, on the opposite sides with respect to the center axis J1. In this manner, the exposed portions 221 of the guide portion 22 can function each as a guiding portion for guiding the blade portion 111. Accordingly, with the present embodiment, the guide member 8 can be omitted. Further, since skin cutting can be performed on the outer sides of the guide portion 22, a cut skin portion 2000 can be formed at nearer portions of the body surface H at which the puncture member 3A is to pass.

Note that, when the guide portion 22 is pressed toward the living body, a portion (space) of the guide portion 22 between the opposite end portions functions as the aforementioned escape portion into which a central portion of the pelvis (for example, the pubis and so forth) and the living body tissue which surrounds the same are admitted.

FIG. 37 is a view depicting a guide portion provided on a medical tool according to a sixth embodiment of the present disclosure.

Although the sixth embodiment of the medical tool of the present disclosure is described below with reference to FIG. 37, the description is given principally of differences of the sixth embodiment from the aforementioned embodiments while description of like matters are omitted herein to avoid redundancy.

The present embodiment is similar to the fifth embodiment except that it is different in configuration of the guide portion.

As depicted in FIG. 37, lacking portions 222 are formed at the opposite end portions of a guide portion 22a such that the outer side thereof lacks to an intermediate portion in the thicknesswise direction of the guide portion 22a. Consequently, in the state in which the opposite end portions of the guide portion 22a are attached to the body surface H, a space is formed between the needle body 35 and each of the guide portions 22a. The blade portion 111 can be inserted into the lacking portion 222 (space) to form a cut skin portion 2000. Note that, when the cut skin portion 2000 is to be formed, the blade portion 111 may be attached to a face 223 extending along the longitudinal direction of the guide portion 22a from within the face of the guide portion 22a which faces the lacking portion 222 and then moved to the body surface H side to form the cut skin portion 2000.

In this manner, the lacking portion 222 (face 223) can function as a guiding portion.

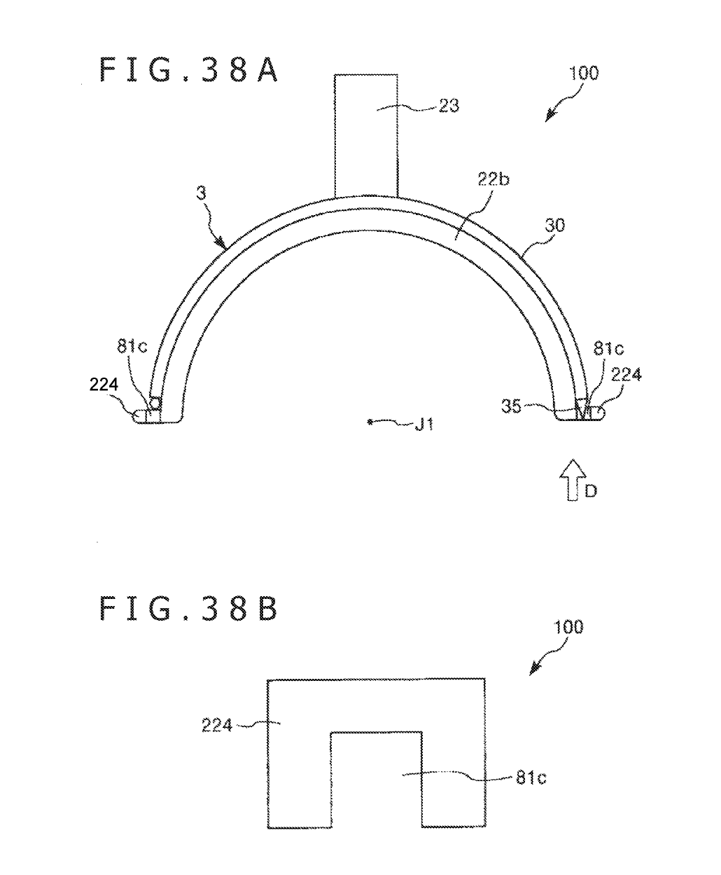

FIG. 38A is a view depicting a guide portion provided on a medical tool according to a seventh embodiment of the present disclosure, and FIG. 38B is a view as viewed in a direction indicated by an arrow mark D in FIG. 38A.

Although the seventh embodiment of the medical tool of the present disclosure is described below with reference to FIGS. 38A and 38B, the description is given principally of differences of the seventh embodiment from the aforementioned embodiments while description of like matters are omitted herein to avoid redundancy.

The present embodiment is similar to the sixth embodiment except that it is different in configuration of the guide portion.

As depicted in FIG. 38A, plate members 224 are provided at the opposite end portions of a guide portion 22b such that it is provided projecting to the outer side of the guide portion 22b. The plate member 224 is configured so as to be attached at one face thereof to the body surface H when the puncture apparatus 1 is to puncture a living body.

Further, as depicted in FIG. 38B, the plate member 224 has a rectangular shape as viewed in plan and has a recess 81c provided at a central portion thereof. The recess 81c is configured from a through-hole extending through the plate member 224 in the thicknesswise direction and is open to one side face of the plate member 224. The recess 81c can function as the aforementioned guiding portion. Further, the puncture member 3 can pass, when it moves rotationally, through the inside of the recess 81c.

Furthermore, with the guide portion 22b, since the plate members 224 are provided at the opposite end portions of the guide portion 22b, the area over which the plate members 224 can attach the body surface H is greater. Consequently, the living body can be stably punctured.

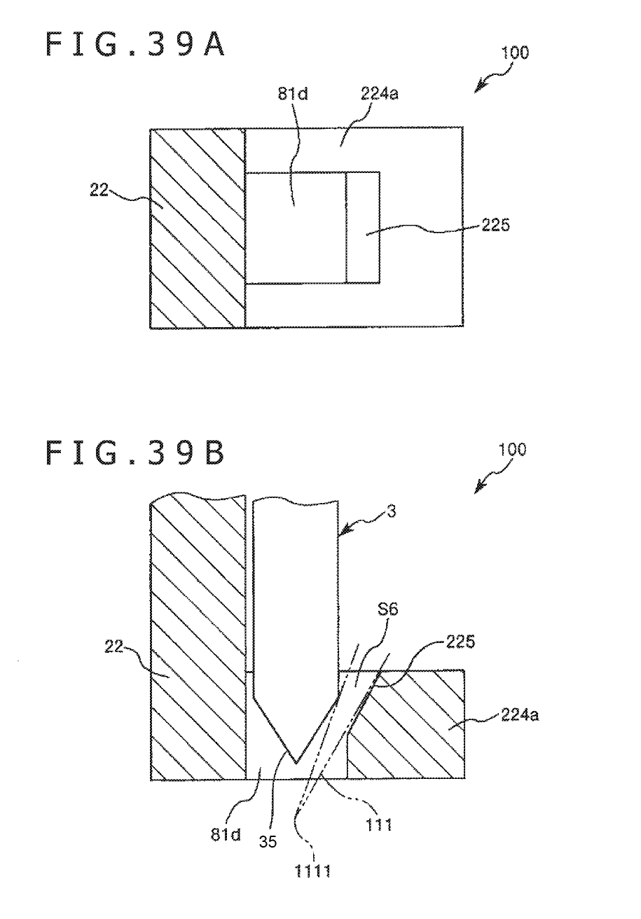

FIG. 39A is a view depicting a plate member of a guide portion provided on a medical tool according to an eighth embodiment of the present disclosure, and FIG. 39B is a sectional view of the plate member depicted in FIG. 39A and depicting a skin cutting operation.

Although the eighth embodiment of the medical tool of the present disclosure is described below with reference to FIGS. 39A and 39B, the description is given principally of differences of the eighth embodiment from the aforementioned embodiments while description of like matters are omitted herein to avoid redundancy.

The present embodiment is similar to the seventh embodiment except that it is different in configuration of the plate member.

As depicted in FIG. 39A, a recess 81d of a plate member 224a has a rectangular shape as viewed in plan. Further, a tapering face 225 is formed at a portion, on the opposite side to the body surface H, of one of four inner side faces of the plate member 224a which is positioned on the outer side of the arc of the guide portion 22. The tapering face 225 is inclined such that the inner diameter of the recess 81d increases away from the body surface H.

As depicted in FIG. 39B, a space S6 is formed between the tapering face 225 and the needle body 35 as a result of formation of the tapering face 225. A cut skin portion 2000 can be formed by inserting the blade portion 111 into the space S6. Further, when a skin cutting operation is performed, it is possible to move the blade portion 111 with the blade portion 111 attached to the tapering face 225.

In this manner, the space S6 and the tapering face 225 can function as a guiding portion for guiding the blade portion 111.

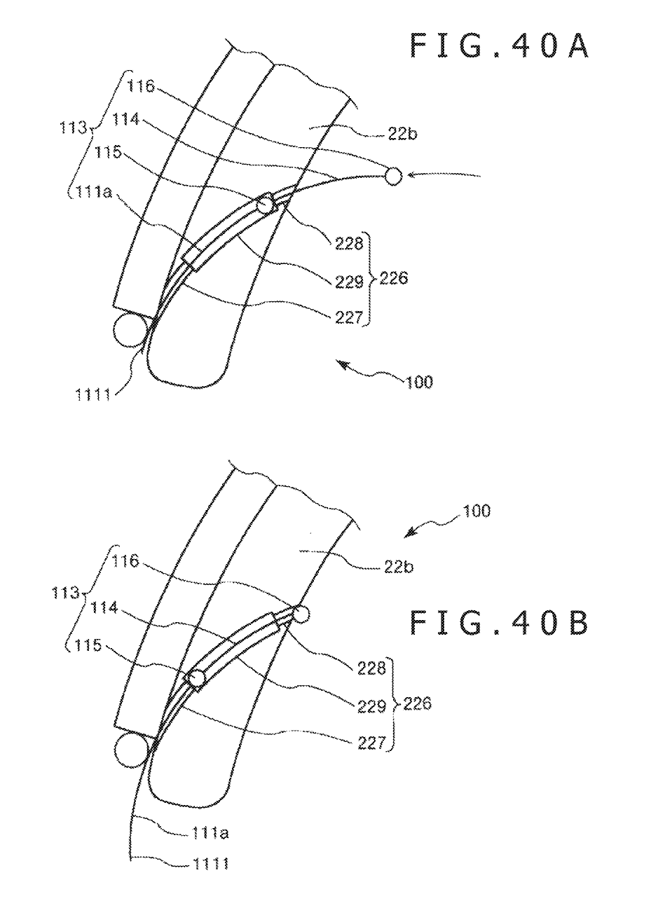

FIG. 40A is a sectional view depicting a guide portion provided on a medical tool according to a ninth embodiment of the present disclosure, and FIG. 40B is a view depicting a procedure of a skin cutting operation.

Although the ninth embodiment of the medical tool of the present disclosure is described below with reference to FIGS. 40A and 40B, the description is given principally of differences of the ninth embodiment from the aforementioned embodiments while description of like matters are omitted herein to avoid redundancy.

The present embodiment is similar to the fifth embodiment except that it is different in configuration of the guide portion.

As depicted in FIGS. 40A and 40B, a through-hole 226 is provided at an end portion of a guide portion 22b such that it extends through the guide portion 22b from the inner side to the outer side. The through-hole 226 has an outer side small diameter portion 227 positioned on the outer side, an inner side small diameter portion 228 positioned on the inner side, and a large diameter portion 229 positioned between the outer side small diameter portion 227 and the inner side small diameter portion 228. The outer side small diameter portion 227, inner side small diameter portion 228 and large diameter portion 229 are communicated with each other. Further, the through-hole 226 is provided in an inclined relationship such that the distance thereof from the end portion of the guide portion 22b decreases in order of the inner side small diameter portion 228, large diameter portion 229 and outer side small diameter portion 227.

A skin cutting portion 113 is inserted for movement in such a through-hole 226 as just described. The skin cutting portion 113 has a blade portion 111a, an operation wire 114 for operating the blade portion 111a, a stopper 115 provided between the blade portion 111a and the operation wire 114, and a grasping portion 116 provided at an end portion of the operation wire 114 on the opposite side to the stopper 115.

The blade portion 111a has a sharp cutting edge 1111 at a distal portion thereof and has a generally rectangular shape whose widthwise direction is a depthwise direction of the plane of FIGS. 40A and 40B.

The operation wire 114 is configured from a belt-like member and has such a suitable degree of rigidity that, when the operation wire 114 is operated to move in the direction indicated by an arrow mark in FIG. 40A, it does not suffer from deformation such as, for example, buckling.

The stopper 115 has a spherical shape and is disposed in the large diameter portion 229. The stopper 115 has a diameter greater than the inner diameter (at least one of the width and the height) of the outer side small diameter portion 227 and the inner side small diameter portion 228. Consequently, the stopper 115 is prevented from entering the outer side small diameter portion 227 and the inner side small diameter portion 228 and can define the limits to the movement of the blade portion 111a.

With such a skin cutting portion 113 as described above, the grasping portion 116 can be grasped to perform a pushing operation in the direction indicated by the arrow mark in FIG. 40A to project the blade portion 111a from the outer side small diameter portion 227 and perform skin cutting. Further, at this time, the blade portion 111a is restricted to the movement limit through the engagement between the stopper 115 and the distal side (outer side small diameter portion 227 side) inner side face of the large diameter portion 229. Consequently, the cutting edge 1111 can be prevented with relative certainty from being inserting into the living body by more than a necessary amount.

Note that, if the grasping portion 116 is grasped and pulled to the opposite direction to the direction indicated by the arrow mark in FIG. 40A, then the blade portion 111a can be retracted to the guide portion 22b side. Upon such a pulling operation as just described, the stopper 115 is restricted to the movement limit through the engagement thereof with the distal side (inner side small diameter portion 228 side) inner side face. Consequently, the skin cutting portion 113 can be prevented from being pulled from the guide portion 22b.

In this manner, with the skin cutting portion 113, a cut skin portion 2000 of an appropriate size can be obtained with relative certainty. Further, a skin cutting operation can be performed relatively safely by a simple configuration, which include the blade portion 111a as described above.

Note that the skin cutting portion 113 is provided at the opposite end portions of the guide portion 22b (not depicted).

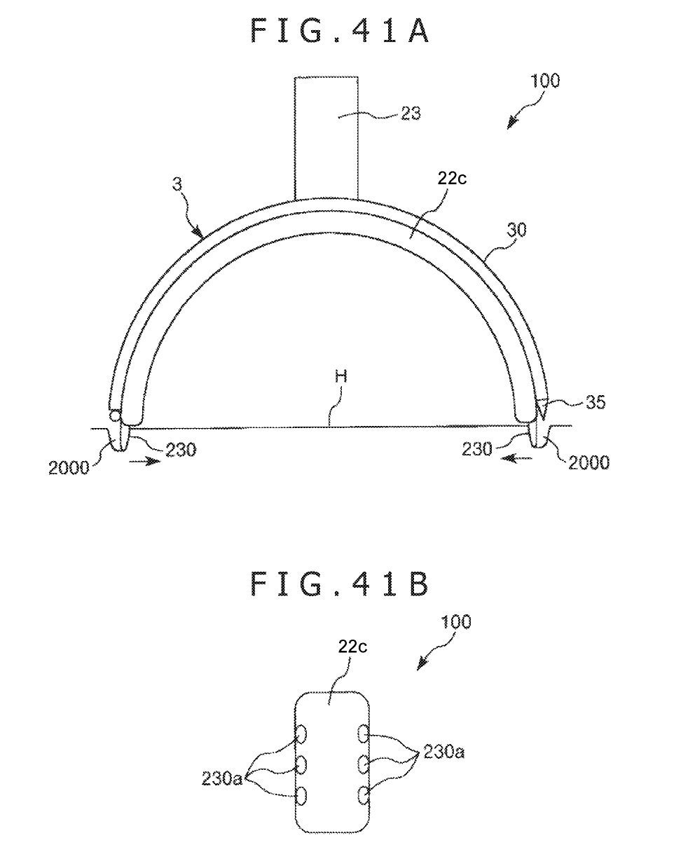

FIG. 41A is a view depicting a guide portion provided on a medical tool according to a tenth embodiment of the present disclosure, and FIG. 41B is a view depicting a modification to a distal portion of the guide portion of FIG. 41A.

Although the tenth embodiment of the medical tool of the present disclosure is described below with reference to FIGS. 41A and 41B, the description is given principally of differences of the tenth embodiment from the aforementioned embodiments while description of like matters are omitted herein to avoid redundancy.

The present embodiment is similar to the first embodiment except that it is different in configuration of the guide portion.

As depicted in FIG. 41A, a pair of insertion portions (expansion portions) 230 are provided at the opposite end portions of a guide portion 22c such that they project in extending directions of the guide portion 22c.

Each of the insertion portions 230 is configured from a tongue provided in a continuing relationship from an outer side face of the guide portion 22c and has a thickness smaller than that of the guide portion 22c.

The insertion portions 230 is attached at the opposite end portions of the guide portion 22c to the body surface H to establish a positioned state, and the guide portion 22c is further pressed against the living body side to enter cut skin portions 2000. Further, where the cut skin portions 2000 are configured a little on the outer sides of the body surface H with respect to the opposite end portions of the guide portion 22c, in the state in which the insertion portions 230 are admitted in the cut skin portions 2000, they expand the cut skin portions 2000 in directions indicated by arrow marks in FIG. 41A. Consequently, when the puncture member 3 is moved rotationally, the puncture member 3 can be inserted into the living body through one of the cut skin portions 2000 with relative certainty and can be projected to the outside of the living body from the other cut skin portion 2000 thereby to form an insertion hole.

FIG. 41B is a view when an end portion of the guide portion 22c is viewed from the body surface H side in FIG. 41A and a modification to the insertion portion 230. An insertion portion 230a depicted in FIG. 41B has a form of a bar, and three such insertion portions 230a are provided on each of an extension line of an outer side face of the guide portion 22c and an extension line of an inner side face of the guide portion 22c. Each of the insertion portions 230a can be rounded at end portions thereof. Further, since the thickness of the insertion portions 230a is smaller than that of the insertion portions 230, an insertion operation thereof into a living body can be performed comparatively easily. Further, the living body tissue can enter between the insertion portions 230a and can be grasped thereby with a higher degree of certainty.

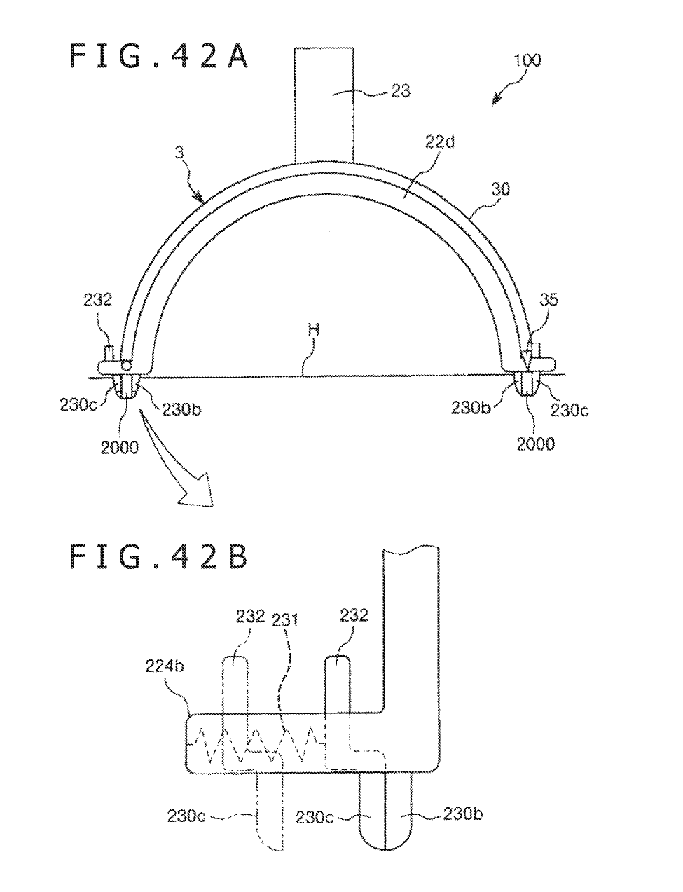

FIG. 42A is a view depicting a guide portion provided on a medical tool according to an eleventh embodiment of the present disclosure, and FIG. 42B is an enlarged view of an end portion of the guide portion of FIG. 42A.

Although the eleventh embodiment of the medical tool of the present disclosure is described below with reference to FIGS. 42A and 42B, the description is given principally of differences of the eleventh embodiment from the aforementioned embodiments while description of like matters are omitted herein to avoid redundancy.

The present embodiment is similar to the tenth embodiment except that it is different in configuration of the guide portion.

As depicted in FIG. 42A, an insertion portion 230b and another insertion portion 230c are provided at the opposite end portions of a guide portion 22d in a continuing relationship to an outer side face of the guide portion 22d. The insertion portions 230b are fixed to the guide portion 22d, and the insertion portions 230c are configured for movement toward and away from the insertion portions 230b. Further, each insertion portion 230c is interlocked with an operation portion 232 for moving the insertion portion 230c toward and away from the insertion portion 230b.

In the following, a configuration of end portions of the guide portion 22d is described. However, since the opposite end portions of the guide portion 22d have configurations similar to each other, one of the end portions of the guide portion 22d is described as a representative.

At an end portion of the guide portion 22d, a plate member 224b is provided and supports the insertion portion 230b and the insertion portion 230c. A through-hole 226 (not depicted) is provided in the plate member 224b such that the puncture member 3 can pass through the same similarly to the aforementioned plate member 224a. Further, a slit is formed which is communicated with the through-hole 226 and allows the insertion portion 230b to move therein.

A tension spring (biasing member) 231 is provided on the plate member 224b such that it is fixed at one end thereof to an inner side face of the slit and fixed at the other end thereof to the insertion portion 230c. Consequently, in a natural state in which no external force is applied, the insertion portion 230c is in a state in which it is spaced away from the insertion portion 230b under the tension of the tension spring 231 as depicted in FIG. 42A.

From this spaced state, force is applied to the operation portion 232 to move the insertion portion 230c toward the insertion portion 230b against the basing force of the tension spring 231 as depicted in FIG. 42B. Consequently, the insertion portion 230b and the insertion portion 230c can assume a state in which they are positioned near to (closely contact with) each other. In this state, the insertion portion 230b and the insertion portion 230c are inserted into a cut skin portion 2000.

Then, the application of the force to the operation portion 232 is canceled. Consequently, the insertion portion 230c is placed back into a state in which it is spaced away from the insertion portion 230b pulled by the tension spring 231. Thereupon, the cut skin portions 2000 are pulled to expand in the opposite directions to each other by the insertion portions 230c which move so as to be spaced away from the insertion portions 230b.

Note that, in the state in which the cut skin portions 2000 are expanded by the insertion portion 230b and the insertion portion 230c, since the through-hole 226 is positioned between the insertion portion 230b and the insertion portion 230c, an insertion hole can be formed in the living body in the state in which the cut skin portions 2000 are expanded.

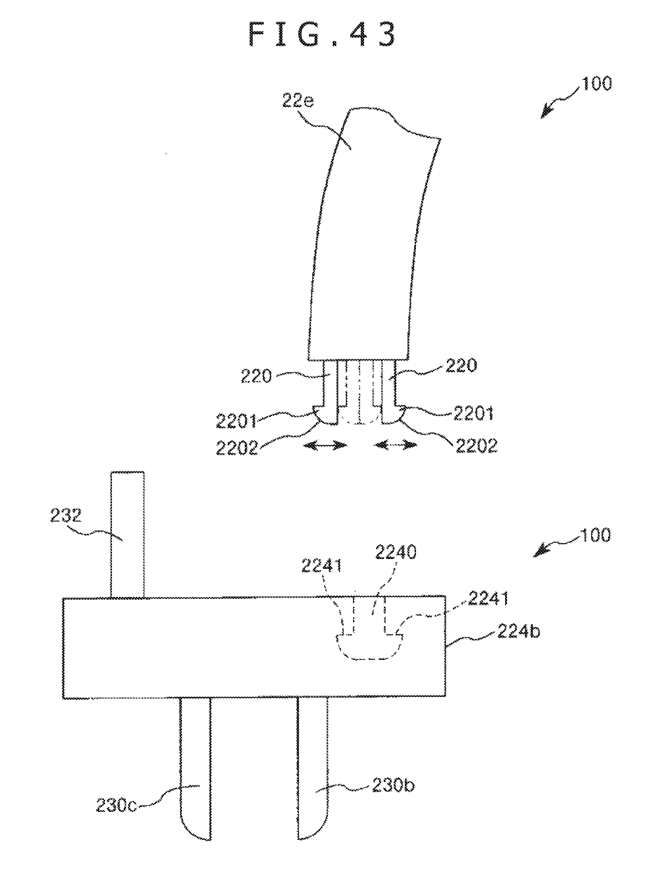

FIG. 43 is a view depicting an end portion of a guide portion provided on a medical tool according to a twelfth embodiment of the present disclosure.

Although the twelfth embodiment of the medical tool of the present disclosure is described below with reference to FIG. 43, the description is given principally of differences of the twelfth embodiment from the aforementioned embodiments while description of like matters are omitted herein to avoid redundancy.

The present embodiment is similar to the eleventh embodiment except that it is different in configuration of the guide portion.

As depicted in FIG. 43, a pair of interlock portions 220 are provided at an end portion of a guide portion 22e. The interlock portions 220 are configured from projecting pieces which project along an extending direction of the guide portion 22e.

A biasing member (not depicted) such as, for example, a spring is provided between the interlock portions 220 and biases the interlock portions 220 in directions in which they are spaced away from each other. In a natural state, the interlock portions 220 are spaced away from each other. However, if external force is applied to the interlock portions 220 against the biasing force of the biasing member, then the interlock portions 220 can be moved toward (closely contacted) with each other as indicated by two-dot chain lines in FIG. 43. In this manner, the interlock portions 220 are configured for movement toward and away from each other.

Further, the interlock portions 220 have anchor portions 2201 formed at the distal portion thereof such that they project to the opposite directions to each other. The anchor portions 2201 have a thickness decreasing toward the distal side and have an outer surface which forms a curved face 2202.

A plate member 224b and the guide portion 22e are formed as separate members from each other. The plate member 224b has a hole portion 2240 open to an upper face thereof. The hole portion 2240 has a steeply expanded portion at a lower end portion thereof such that a stepped portion 2241 is formed on an inner side face of the hole portion 2240.

If the guide portion 22e is moved toward the plate member 224b configured in such a manner as just described and the interlock portions 220 are moved toward the hole portion 2240, then the curved faces 2202 of the interlock portions 220 are attached to each other. If, in the attached state, the guide portion 22e is pressed against the plate member 224b further, then an edge portion of the opening of the hole portion 2240 slidably moves along the curved faces 2202. At this time, the interlock portions 220 move in directions in which they approach each other against the biasing force of the biasing member positioned between them. Then, when the interlock portions 220 are closely contacted with each other, they can move in the hole portion 2240. Note that, when the interlock portions 220 move in the hole portion 2240, while the interlock portions 220 are biased in directions in which they are spaced away from each other by the biasing member, the movement away from each other is restricted by the inner side face of the hole portion 2240. The guide portion 22e is further pressed against the plate member 224b to complete the insertion of the interlock portions 220 into the hole portion 2240. Since the lower end face of the hole portion 2240 has a steeply expanded diameter portion, in a state in which the insertion of the interlock portions 220 into the hole portion 2240 is completed, the interlock portions 220 can move away from each other. In the state in which the interlock portions 220 are spaced away from each other, since the anchor portions 2201 of the interlock portions 220 engage with the stepped portions 2241, the guide portion 22e and the plate member 224b are interlocked with each other.

Note that, for example, if a knob or the like capable of operating the interlock portions 220 from the outside of the guide portion 22e is provided, then the interlocking of the guide portion 22e and the plate member 224b with each other can be readily canceled.

Where the guide portion 22e and the plate member 224b are configured in a freely detachable manner from each other in this manner, the form of the puncture apparatus 1 can be selected as occasion demands.

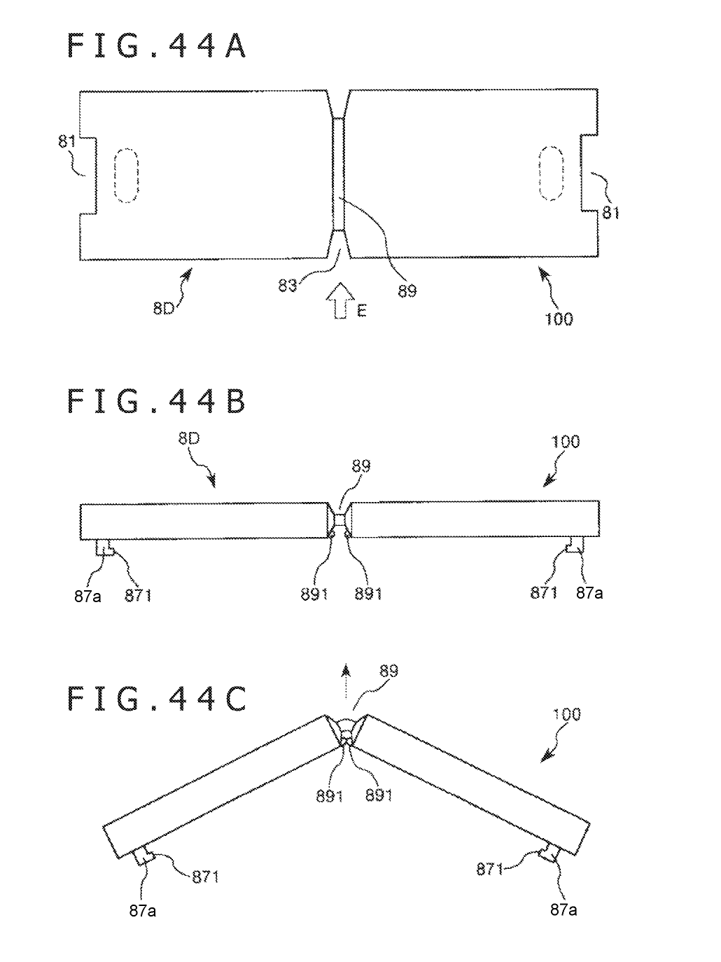

FIGS. 44A to 44C depict an end portion of a guide portion provided on a medical tool according to a thirteenth embodiment of the present disclosure.

Although the thirteenth embodiment of the medical tool of the present disclosure is described below with reference to FIGS. 44A to 44C, the description is given principally of differences of the thirteenth embodiment from the aforementioned embodiments while description of like matters are omitted herein to avoid redundancy.

The present embodiment is similar to the first embodiment except that it is different in configuration of the guide member.

As depicted in FIG. 44A, a guide member 8D has a reduced thickness portion 89 provided at a central portion thereof in the longitudinal direction so as to extend along the widthwise direction at a central portion of the guide member 8D. Consequently, the guide member 8D can be bent at the central portion in the longitudinal direction thereof using the reduced thickness portion 89.

Further, as depicted in FIG. 44B, in the proximity of the reduced thickness portion 89 of the guide member 8D, a pair of projections 891 are provided in an opposing relationship to each other with the reduced thickness portion 89 interposed therebetween. Consequently, when the guide member 8D is bent as depicted in FIG. 44B, the projections 891 can be attached to each other to restrict the guide member 8D from being bent any more.

Further, on the lower face of the guide member 8D, a pair of plate pieces 87a are provided in the proximity of the recesses 81 and formed in a projecting manner to the lower side as depicted in FIGS. 44B and 44C. The plate pieces 87 have projections 871 at an end portion thereof so as to project in directions toward each other.

After two cut skin portions 2000 are formed using such a guide member 8D as described above, the plate pieces 87a are inserted into the cut skin portions 2000. Then, the guide member 8D is grasped and lifted in a direction spaced away from the body surface H while being bent. At this time, since the projections 871 are caught by the living body tissue at peripheral portions of the cut skin portions 2000, also the living body tissue at the peripheral portions of the cut skin portions 2000 are pulled upwardly a little as depicted in FIG. 44C. As a result, the cut skin portions 2000 are expanded. In this manner, each of the projections 871 can function as a catching portion for catching the living body tissue at a peripheral portion of the cut skin portion 2000.

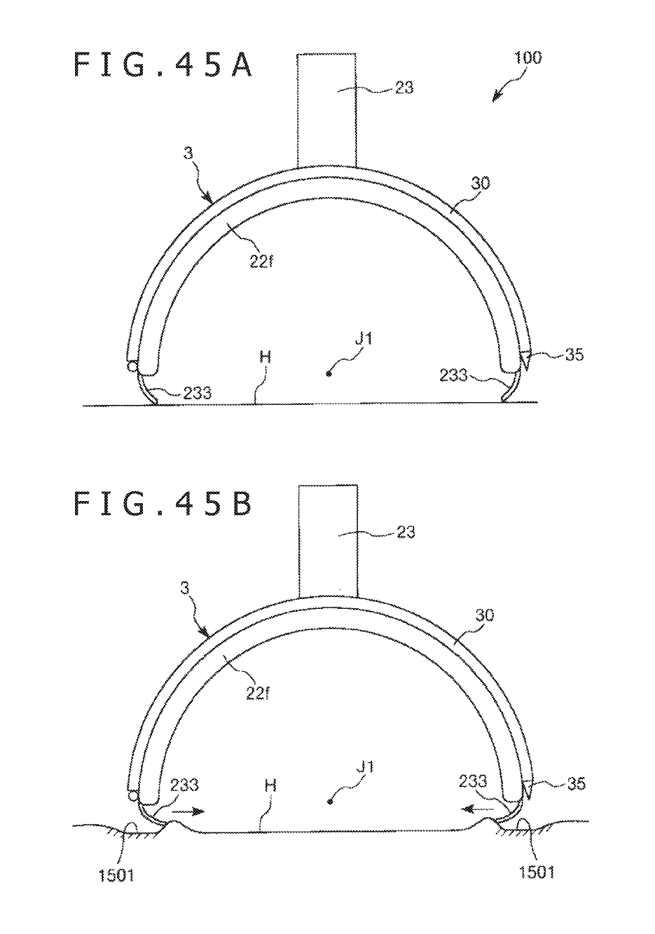

FIGS. 45A and 45B are views depicting a medical tool according to a fourteenth embodiment of the present disclosure and illustrating an operation procedure of the medical tool.

Although the fourteenth embodiment of the medical tool of the present disclosure is described below with reference to FIGS. 45A and 45B, the description is given principally of differences of the fourteenth embodiment from the aforementioned embodiments while description of like matters are omitted herein to avoid redundancy.

The present embodiment is substantially similar to the first embodiment except that it is different in configuration of the puncture apparatus.

As depicted in FIGS. 45A and 45B, tongues (attachment portions) 233 are provided at the opposite end portions of a guide portion 22f. Since the tongues 233 have configurations similar to each other, one of the tongues 233 is described below representatively.

The tongue 233 is configured from a plate piece having a width in the depthwise direction to the plane of FIGS. 45A and 45B. The tongue 233 is inclined with respect to the extending direction of the guide portion 22f such that the distal portion thereof approaches the center axis J1. Further, the tongue 233 has elasticity and is curved toward the inner side of the guide portion 22f.

Now, a puncture method of the present embodiment is described.

When the living body is to be punctured by the puncture member 3, the guide portion 22f is caused to approach the living body first until the tongues 233 are attached to the body surface H (refer to FIG. 45A). In this state, the guide portion 22f is pressed toward the living body side. At this time, the distal portions of the tongues 233 are elastically deformed in directions in which they approach each other while remaining attached to the body surface H as depicted in FIG. 45B. Consequently, portions through which the puncture member 3 passes (portions at which puncture is to be started) from within the body surface H and peripheral portions (portions to which slanting lines are applied in FIG. 45B) 1501 are pulled in the direction indicated by arrow marks so as to be placed into a tensioned state. In the tensioned state, the puncture member 3 can easily puncture the portion 1501.

In this manner, the tongues 233 function as a tensioning portion for applying force to the body surface H in a direction different from a direction in which the puncture member 3 travels to establish a tensioned state. Further, as described above, the tongues 233 are provided in an inclined relationship with respect to the extending direction of the guide portion 22f. Consequently, the medical tool of the present embodiment can be attached to the body surface H and elastically deformed with relative certainty, and the deformation direction can be controlled with relative certainty. Further, since the tongues 233 are deformed so as to approach each other toward the center axis J1 side, namely, toward the inner side of the guide portion 22f, the puncture member 3 can be prevented from moving rotationally to puncture the living body.

With such tongues 233 as described above, puncture of the living body can be easily performed for a patient whose body surface (skin) is loosened to make puncturing comparatively difficult such as, for example, senior persons.

Further, where cut skin portions 2000 are formed, the tongues 233 can function as expansion portions for pulling peripheral portions of the cut skin portions 2000 from within the body surface H to expand the cut skin portions 2000 (not depicted).

Note that the tongues 233 may be formed integrally with the guide portion 22f or may be configured as separate members from the guide portion 22f. Further, where the tongues 233 are configured as separate members from the guide portion 22f, they may be configured in a freely detachable manner from the guide portion 22f.

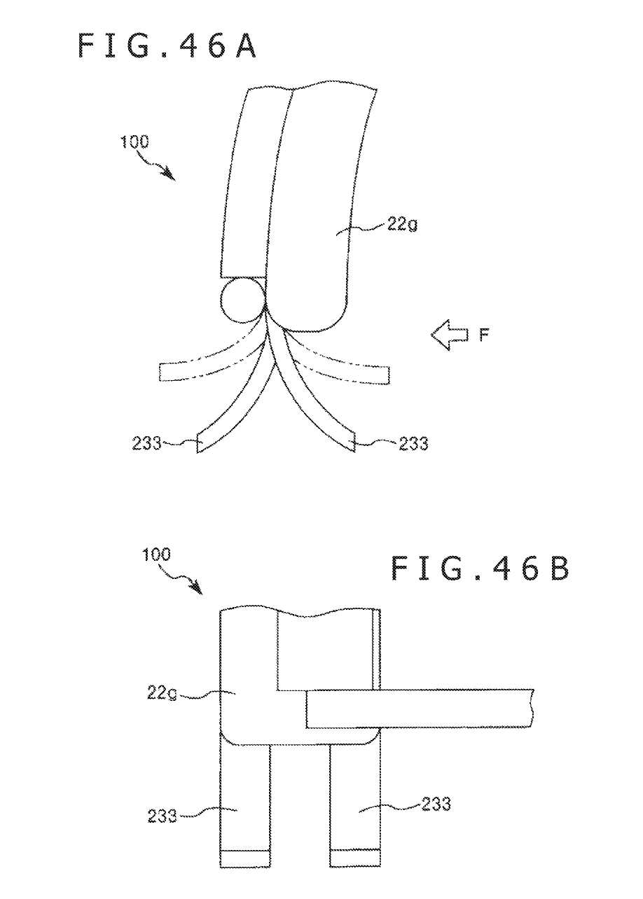

FIG. 46A is a view depicting a medical tool according to a fifteenth embodiment of the present disclosure, and FIG. 46B is a view as viewed in the direction indicated by an arrow mark F in FIG. 46A.

Although the fifteenth embodiment of the medical tool of the present disclosure is described below with reference to FIGS. 46A and 46B, the description is given principally of differences of the fifteenth embodiment from the aforementioned embodiments while description of like matters are omitted herein to avoid redundancy.

The present embodiment is similar to the fourteenth embodiment except that it is different in configuration of the tongues.

As depicted in FIG. 46A, a pair of tongues 233 are provided on a guide portion 22g. The tongues 233 are provided in a spaced relationship from each other at positions different from each other of an end portion of the guide portion 22g. Further, the distance between the tongues 233 is equal to or greater by little than the width of the puncture member 3. Further, the tongues 233 are inclined in directions opposite to each other in a natural state thereof.

When the tongues 233 having such a configuration as described above are pressed against the body surface H, since they are inclined in directions opposite to each other in a natural state thereof, they are elastically deformed so as to be spaced away from each other in the directions opposite to each other. At this time, since the body surface H is pulled toward the sides opposite to each other (directions different from each other), portions through which the puncture member 3 passes from within the body surface H can be placed into a more tensioned state than that of the portion 1501 in the aforementioned fourteenth embodiment. Consequently, puncture of the body surface H can be performed with a relatively higher degree of easiness.

Further, since the separated distance between the tongues 233 is equal to or greater than the width of the puncture member 3 as described above, the puncture member 3 can be caused to pass (puncture) between the tongues 233 while a more tensioned state of the portions through which the puncture member 3 passes from within the body surface H is maintained by the tongues 233.

FIG. 47 is a view depicting a medical tool according to a sixteenth embodiment of the present disclosure.

Although the sixteenth embodiment of the medical tool of the present disclosure is described below with reference to FIG. 47, the description is given principally of differences of the sixteenth embodiment from the aforementioned embodiments while description of like matters are omitted herein to avoid redundancy.

The present embodiment is similar to the fifteenth embodiment except that it is different in configuration of the tongues.

As depicted in FIG. 47, another pair of tongues 234 can be provided at an end portion of a guide portion 22h in addition to the pair of tongues 233 of the fifteenth embodiment. The tongues 234 are provided so as to oppose to each other in the widthwise direction of the guide portion 22h. Further, the tongues 234 are provided so as to be inclined in directions in which the distal portions thereof are spaced away from each other.

With such a configuration as described above, in a tensioned state, a portion of the body surface H through which the puncture member 3 passes is pulled in four directions different from each other. Consequently, a state in which the body surface H is further strained can be established and rotary movement of the puncture member 3 can be performed in more stability.

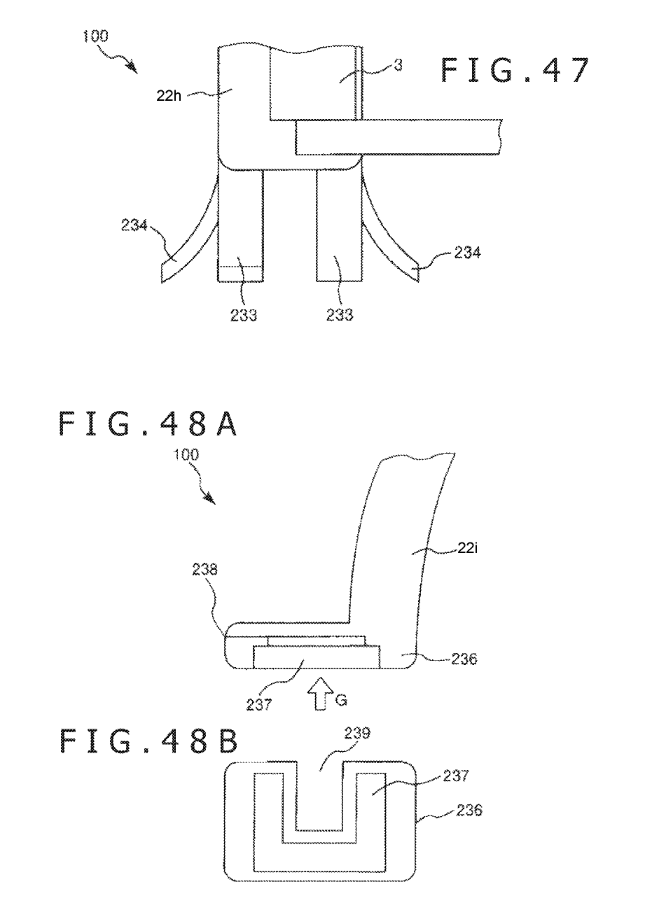

FIG. 48A is a view depicting a medical tool according to a seventeenth embodiment of the present disclosure, and FIG. 48B is a view as viewed in the direction indicated by an arrow mark G in FIG. 48A.

Although the seventeenth embodiment of the medical tool of the present disclosure is described below with reference to FIGS. 48A and 48B, the description is given principally of differences of the seventeenth embodiment from the aforementioned embodiments while description of like matters are omitted herein to avoid redundancy.

The present embodiment is similar to the sixteenth embodiment except that it is different in configuration of the guide portion.

As depicted in FIGS. 48A and 48B, a suction portion 235 as a tensioning portion is provided at an end portion of a guide portion 22i. The suction portion 235 can include a plate-like portion 236, a suction hole 237 provided on the plate-like portion 236 and a suction port 238 connected to the suction hole 237.

The plate-like portion 236 is provided in a projecting manner in a direction crossing with the extending direction of the guide portion 22i. The plate-like portion 236 has a rectangular shape as viewed in plan, and the lower face of the plate-like portion 236 is a face for being attached to the body surface H. Further, the plate-like portion 236 has a recess 239 open to one side face thereof. Consequently, when the puncture member 3 is moved rotationally, the puncture member 3 can pass through the inside of the recess 239.

The suction hole 237 is configured from a recess open to the lower face of the plate-like portion 236 and is formed along the shape of the plate-like portion 236. Further, a suction device such as a pump can be connected to the suction port 238 and, if the suction device is rendered operative in a state in which the lower face of the plate-like portion 236 is attached to the body surface H, then the body surface H can be sucked. In the state in which the body surface H is sucked, the body surface H is pulled to the upper side in FIG. 48A so as to be placed in a tensioned state in a direction different from a direction in which puncture is performed by the puncture member 3, namely, in a planar direction of the body surface H.

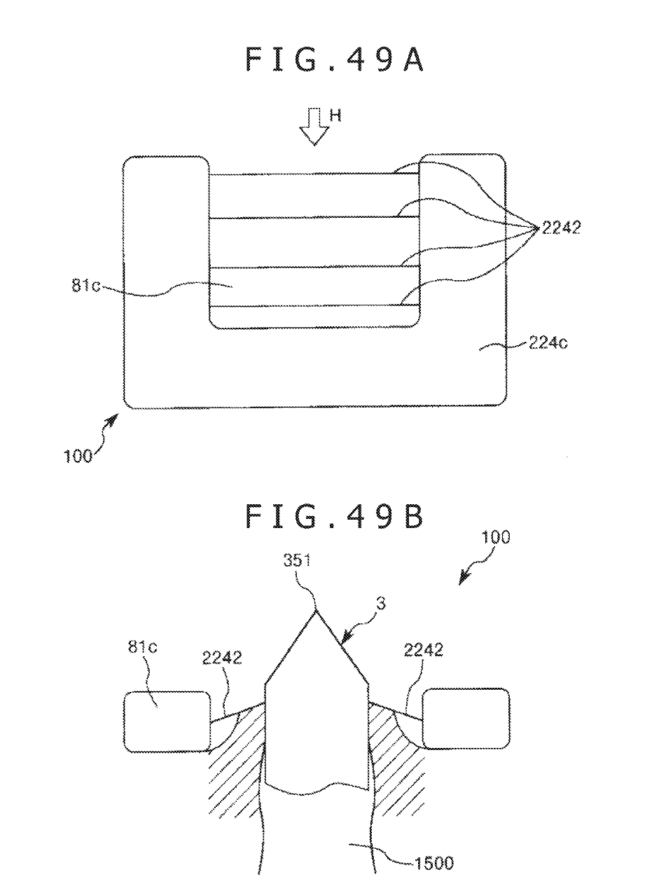

FIG. 49A is a view depicting a medical tool according to an eighteenth embodiment of the present disclosure, and FIG. 49B is a view as viewed in the direction indicated by an arrow mark H in FIG. 49A.

Although the eighteenth embodiment of the medical tool of the present disclosure is described below with reference to FIGS. 49A and 49B, the description is given principally of differences of the eighteenth embodiment from the aforementioned embodiments while description of like matters are omitted herein to avoid redundancy.

The present embodiment is similar to the seventh embodiment except that it is different in configuration of the plate member.

As depicted in FIG. 49A, a plurality of (in the present embodiment, four) wires (deformation suppressing portions) 2242 are provided in a recess 81c of a plate member 224c. The wires 2242 are fixed at the opposite ends thereof to a pair of inner side faces opposing to each other in the widthwise direction of the guide portion 22b from among inner side faces of the recess 81c. Further, each of the wires 2242 is configured from an elastic body and can be deflected and elastically deformed when external force is applied thereto.

In the present embodiment, such a plate member 224c as described above is provided at an end portion on the opposite side to a needle tip 351 in an initial state from between the opposite end portions of the guide portion 22b. Note that the plate member 224c may be provided at the opposite ends of the guide portion 22b.

When the puncture member 3 punctures a living body and then punctures the body surface H from within the living body and then passes through the body surface H again, a peripheral portion of the portion at which the puncture member 3 passes through the living body from within the body surface H is deformed so as to swell toward the outer side of the living body (advancing direction of the puncture member 3). However, as depicted in FIG. 49B, the swollen body surface H is restricted from moving to the upper side with respect to the wires 2242 by the wires 2242. Consequently, excessive deformation of the living body tissue at a peripheral portion of a wound by the puncture member 3 can be suppressed or prevented. As a result, an insertion hole can be formed with lower invasion.

Further, when the body surface H is attached to the wires 2242 and is restricted from moving to the upper side, the wires 2242 can be deflected and deformed. Consequently, damage to the body surface H by the wires 2242 can be prevented.

Note that, when the puncture member 3 passes between the wires 2242, the wires 2242 are deflected and deformed in a direction toward this side and the depthwise direction with respect to the plane of FIG. 49B along the shape of the puncture member 3.

In this manner, the wires 2242 can function as deformation preventing portions capable of preventing the body surface H from being deformed together with puncture by the puncture member 3.

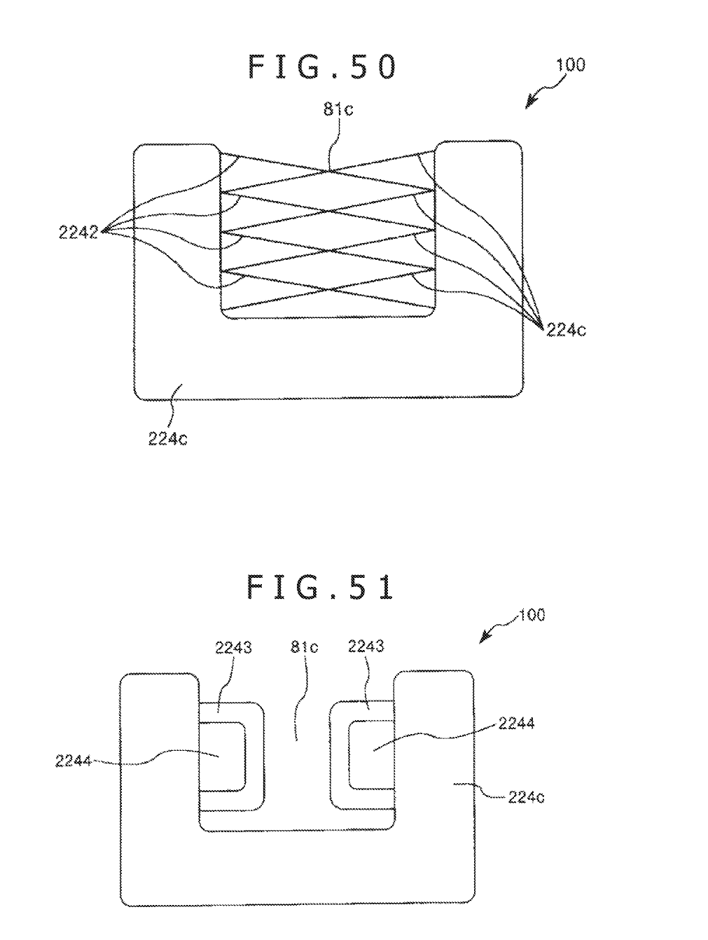

FIG. 50 is a view depicting a medical tool according to a nineteenth embodiment of the present disclosure.

Although the nineteenth embodiment of the medical tool of the present disclosure is described below with reference to FIG. 50, the description is given principally of differences of the nineteenth embodiment from the aforementioned embodiments while description of like matters are omitted herein to avoid redundancy.

The present embodiment is similar to the eighteenth embodiment except that it is different in configuration of the wires.

As depicted in FIG. 50, the plurality of wires 2242 of the plate member 224c are disposed so as to configure a mesh braid. Consequently, since the separated distance between mesh portions is smaller than the separated distance between the wires 2242 adjacent each other of the eighteenth embodiment, the body surface H can be prevented with more certainty from being swollen together with the puncture member 3. Further, since the area in which the plate member 224c contacts with the body surface H swollen together with the puncture member 3 is comparatively great, pressing force by the wires 2242 can be dispersed. Consequently, the swollen body surface H can be prevented from being damaged by the wires 2242.

FIG. 51 is a view depicting a medical tool according to a twentieth embodiment of the present disclosure.

Although the twentieth embodiment of the medical tool of the present disclosure is described below with reference to FIG. 51, the description is given principally of differences of the twentieth embodiment from the aforementioned embodiments while description of like matters are omitted herein to avoid redundancy.

The present embodiment is similar to the eighteenth embodiment except that it is different in configuration of the wires.

As depicted in FIG. 51, on the plate member 224c, elastic pieces 2243 are provided on a pair of inner side faces opposing to each other in the widthwise direction of the guide portion 22b from among inner side faces of the recess 81c.

Each of the elastic pieces 2243 has elasticity and attaches the body surface H swollen together with the puncture member 3 so as to restrict swelling of the body surface H. Note that the elastic pieces 2243 can be deflected and deformed similarly to the aforementioned wires 2242. Further, the puncture member 3 can pass between the elastic pieces 2243.

Further, on the inner side faces of the recess 81c, auxiliary pieces 2244 are provided at portions corresponding to the upper sides of the elastic pieces 2243. Also each of the auxiliary pieces 2244 has elasticity and prevents excessive deformation principally when the elastic pieces 2243 are deformed. In particular, the auxiliary pieces 2243 function as auxiliary portions for applying, when the elastic pieces 2243 are deformed, suitable rigidity to the elastic pieces 2243 and assisting prevention of deformation of the body surface H together with puncture by the puncture member 3.

With such a configuration as just described, the body surface H can be prevented from being deformed together with puncture by the puncture member 3.

The material for configuring the elastic pieces 2243 and the auxiliary pieces 2244 is not restricted particularly, and various kinds of rubber materials, various kinds of resin materials or the like can be used.

Although the medical tool and the puncture method of the present disclosure have been described in connection with the embodiments depicted in the drawings, the present disclosure is not limited to them, and the components in the embodiments can be replaced by arbitrary members having similar functions. Further, some other arbitrary member or members may be added to the present disclosure. Further, the embodiments may be combined suitably.

Note that, while two cut skin portions are formed in the embodiments, according to the present disclosure, the number of such cut skin portions is not limited to two, but only it is necessary for at least one cut skin portion to be provided in a region in which puncture by the puncture member is to be started.

Further, while a configuration in which the main body can be separated into a distal end divisional piece and a proximal end divisional piece is described in the aforementioned embodiments, the configuration of the main body is not limited to this, and a configuration that the main body cannot be separated into a distal end divisional piece and a proximal end divisional piece may be applied. In particular, the main body may be configured in the form of a single tube. In this case, also the state maintaining portion is omitted.

Further, while the sheath in one of the aforementioned embodiments is configured as part of the puncture member, the sheath is not limited to this. In particular, the puncture member may be used such that a sheath is inserted in a through-hole formed in the living body using some means. Describing the method given just above particularly in a corresponding relationship to the aforementioned first embodiment, the puncture apparatus 1 in which the puncture member 3 is omitted is prepared, and the distal portion 711 of the insertion portion 71 is caused to puncture, using the insertion portion 71 as the puncture member, the right side inguinal region of the patient. Then, the distal portion 711 is caused to pass one of the obturator foramens, the space between the urethra and the vagina, and the other one of the obturator foramens in order until the insertion portion is projected from the left side inguinal region to the outside of the body. Then, the insertion portion 71 is inserted into the inside of the puncture apparatus 1 and the sheath 30 (main body 31) is advanced to the inside of the body along the insertion portion 71 until a state in which the opposite ends thereof are projected from the body surface H is established. Then, the insertion portion 71 is removed from the inside of the body. Consequently, the sheath 30 is disposed into the living body. Then, the implant main body 91 is disposed into the sheath 30 and the sheath 30 is pulled off from within the body. The implant main body 91 can be indwelled thereby in the living body similarly as in the aforementioned embodiments.

Further, for example, the distal portion 711 of the insertion portion 71 is caused to puncture the right side inguinal region of the patient and then pass one of the obturator foramens, the space between the urethra and the vagina, and the other one of the obturator foramens in order until the distal portion 711 is projected from the left side inguinal region to the outside of the body. Thereafter, the distal portion of the sheath 30 is fixed to the distal portion 711. Then, the insertion portion 71 is rotated inversely and removed from the inside of the body so that the sheath 30 is indwelled in the living body. Then, if the implant main body 91 is disposed in the sheath 30 and then the sheath 30 is pulled out from within the body while the implant main body 91 is left in the living body, then the implant main body 91 can be indwelled in the living body similarly as in the aforementioned embodiments.

Further, in the aforementioned embodiments, a configuration is described that the implant main body is inserted into the main body after the main body of the puncture member is disposed into the living body, the embodiment is not limited to this, and the implant main body may be accommodated in the puncture member (main body) from the beginning. In this case, for example, it is preferable to fix a string positioned on the needle tip side from between two strings, which the implant main body has to the needle tip in advance. This makes it possible to project, when the needle tip is removed from the main body, the string to the outside of the main body together with the removal of the needle tip. Therefore, fine adjustment of the disposition of the implant main body after the process just described or the like can be performed smoothly.

Further, while a case in which the puncture apparatus is applied to a device for use in embedding in a living body an embeddable implant for treatment of female urinary incontinence has been described in the aforementioned embodiments, the application of the puncture apparatus is not limited to the described one.

For example, the target of the application of the present disclosure can include excretory disorders attendant on the weakening of the pelvic floor muscle group (urinary urgency, frequent urination, urinary incontinence, fecal incontinence, urinary retention, strangury and so forth) and pelvic floor disorders including pelvic organ prolapse, vesicovaginal fistula, urethrovaginal fistula, pelvic pain or the like. The pelvic organ prolapse can include disorders of cystocele, enterocele, rectocele, hysterocele and so forth. Alternatively, the pelvic organ prolapse can include such disorders as anterior vaginal prolapse, posterior vaginal prolapse, vaginal apical prolapse, vaginal vault prolapse and the like in which the naming method thereof is due to the prolapsed vaginal-wall part.

Further, the overactive tissues include the bladder, vagina, uterus, bowel and the like. The less active tissues include bones, muscles, fascias, ligaments and the like. In particular, in the case of pelvic floor disorders, the less active tissues include an obturator fascia, a coccygeus fascia, a cardinal ligament, an uterosacral ligament, a sacrospinous ligament and the like.

The procedure for interlocking an overactive tissue with a less active tissue in the pelvic floor disorder, can include retropubic sling surgery, transobturator sling surgery (transobturator tape (TOT) surgery), tension-free vaginal mesh (TVM) surgery, uterosacral ligament suspension (USLS) surgery, sacrospinous ligament fixation (SSLF) surgery, iliococcygeus fascia fixation surgery, coccygeus fascia fixation surgery and the like.

The medical tool of the present disclosure has a tensioning portion configured to apply, before a puncture needle for puncturing a living body tissue forms, in the living body tissue, an insertion hole into which an implant can be inserted, force to a portion through which the puncture needle is to pass in a direction different from a direction in which the puncture needle punctures the living body tissue to establish a tensioned state. Therefore, the implant can be embedded easily into the living body, and besides the burden on the patient is light and the safety of the patient is high.

Accordingly, the medical tool of the present disclosure has industrial applicability.

The detailed description above describes a medical tool and a puncture method. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A medical tool, comprising
a tensioning portion configured to apply, before a puncture needle puncturing a living body tissue forms, in the living body tissue, an insertion hole into which an implant can be inserted, force to a portion through which the puncture needle is to pass in a direction different from a direction in which the puncture needle punctures the living body tissue to establish a tensioned state.

2. The medical tool according to claim 1, wherein the tensioning portion includes an attachment portion configured to be attached to a peripheral portion of a portion at which puncture by the puncture needle is to be started to press the peripheral portion to tension the portion at which puncture by the puncture needle is to be started.

3. The medical tool according to claim 2, wherein a plurality of attachment portions press the peripheral portion individually in directions different from each other.

4. The medical tool according to claim 2, wherein the attachment portion is configured from a projecting member having elasticity.

5. The medical tool according to claim 1, wherein the tensioning portion has a suction portion configured to suck a portion through which the puncture needle is to pass.

6. The medical tool according to claim 1, further comprising
a deformation suppressing portion configured to be attached to a living body surface to prevent and suppress deformation of the living body tissue at a peripheral portion of a portion at which the puncture needle is to pass the living body.

7. The medical tool according to claim 6, wherein the deformation suppressing portion is configured from an elastic body configured to be attached, after the puncture needle punctures the living body tissue, again to the living body tissue at a peripheral portion of a portion at which the puncture needle is exposed from the living body surface.

8. A puncture method for puncturing a living body tissue by a puncture needle, which is capable of puncturing the living body tissue, to form an insertion hole into which an implant can be inserted, the method comprising:
applying force to a portion of a living body surface through which the puncture needle is to pass in a direction different from a direction in which the puncture needle punctures to establish a tensioned state; and
puncturing the living body surface placed in the tensioned state by the puncture needle.

9. The puncture method according to claim 8, comprising:
establishing the tensioned state with a tensioning portion on a medical tool, the tensioning portion including an attachment portion configured to be attached to a peripheral portion of a portion at which puncture by the puncture needle is to be started.

10. The puncture method according to claim 9, comprising:
establishing a plurality of tensioned states, each of the plurality of tensioned states being in directions different from each other.

11. The puncture method according to claim 10, comprising:
forming the plurality of tensioned states with a plurality of attachment portions, each of the plurality of attachment portions configured to press the peripheral portion in the different directions.

12. The puncture method according to claim 9, wherein the attachment portion is a projecting member having elasticity.

13. The puncture method according to claim 9, wherein the tensioning portion has a suction portion configured to suck a portion through which the puncture needle is to pass.

14. The puncture method according to claim 9, comprising
a deformation suppressing portion configured to be attached to a living body surface to prevent and suppress deformation of the living body tissue at a peripheral portion of a portion at which the puncture needle is to pass the living body.

15. The puncture method according to claim 14, wherein the deformation suppressing portion is configured from an elastic body configured to be attached, after the puncture needle punctures the living body tissue, again to the living body tissue at a peripheral portion of a portion at which the puncture needle is exposed from the living body surface.
